(19) United States
(12) Patent Application Publication
(10) Pub. No.: US 2008/0274091 A1
(43) Pub. Date: Nov. 6, 2008

(54) Autologous T Cell Manufacturing Processes

(75) Inventors: Vladimir Slepushkin, Damascus, MD (US); Laurent Humeau, Germantown, MD (US)

Correspondence Address: VIRxSYS;c/o MOFO SD, 12531 HIGH BLUFF DRIVE, SUITE 100, SAN DIEGO, CA, 92130-2040, US

(73) Assignee: VIRxSYS Corporation, Gaithersburg, MD (US)

(21) Appl. No.: 10/587,437

(22) PCT Filed: May 22, 2006
(86) PCT No.: PCT/US06/19709

§ 371 Date: Jul. 3, 2008

Related U.S. Application Data
(60) Provisional application No. 60/683,527, filed on May 20, 2005.

(51) Int. Cl. A61K 35/12; C12N 15/87; C12M 1/00; A61P 37/00

(52) U.S. Cl. 424/93.21; 435/457; 435/289.1

(57) ABSTRACT

The invention provides novel processes for manufacturing autologous T cells, transducing T cells and expanding the transduced T cell population.

### RELATED APPLICATIONS

This application is related to U.S. provisional application Ser. No. 60/683,527, filed May 20, 2005, which is incorporated herein by reference in its entirety.

The contents of these documents are incorporated herein by reference.

### TECHNICAL FIELD

This invention relates generally to virology, cell biology and biotechnology. In particular, the invention provides novel processes for manufacturing primary cells, transducing primary cells and expanding the primary cell population.

The present invention is directed to methods, as well as compositions related thereto, for the efficient and stable transduction of cells using viral vectors. The methods result in an increase in the number of transduced cells. The transduced cells can be used for both laboratory and clinical applications.

The present invention is directed to methods, as well as compositions and systems related thereto, for the efficient and stable transduction of cells using viral vectors. The methods increase the efficiency of transduction by, for example, contacting the cell to be transduced with one or more molecules that bind the cell surface. The contacting step may occur before, after, or simultaneously with, introduction of the viral vector to the cells. The methods also result in a larger number of primary cells being cultured and/or grown, by culturing the cells in a multilayer vessel or flask that is able to contain a larger number of cells than a single layered vessel or flask. The present invention also concerns the use of the stably transduced cells in other applications, including expression of nucleic acids borne by the vector or therapy of living organisms.

### BACKGROUND

Barry, S. C. et al. (2000) “Lentiviral and murine retroviral transduction of T cells for expression of human CD40 ligand” Human Gene Therapy 11:323-332. Costello, E. et al. (2000) “Gene transfer into stimulated and unstimulated T lymphocytes by HIV-1-derived lentiviral vectors” Gene Therapy 7:596-604. Douglas, J. et al. (1999) “Efficient transduction of human lymphocytes and CD34+ cells via human immunodeficiency virus-based gene transfer vectors” Human Gene Therapy 10:935-945. Follenzi, A. et al. (2000) “Gene transfer by lentiviral vectors is limited by nuclear translocation and rescued by HIV-1 pol sequences” Nature Genetics 25:217-222. Han, W. et al. (2000) “A soluble form of human Delta-like-1 inhibits differentiation of hematopoietic progenitor cells” Blood 95:1616-1625. Haas, D. L., et al. (2000) “Critical factors influencing stable transduction of human CD34+ cells with HIV-1-derived lentiviral vectors” Molecular Therapy 2:71-80. Hooijberg E. et al. (2000) “NFAT-controlled expression of GFP permits visualization and isolation of antigen-stimulated primary human T cells” Blood 96:459-466. Kishimoto, T. (ed). Leucocyte Typing VI: White Cell Differentiation Antigens Proceedings of the Sixth International Workshop and Conference Held in Kobe, Japan, 10-14 Nov. 1996. Garland Publishing, New York, 1998. Klebba, C. et al. (2000) “Retrovirally expressed anti-HIV ribozymes confer a selective survival advantage on CD4+ T cells in vitro” Gene Therapy 7:408-416. Koc, O. N., et al. (1999) “Transfer of drug resistance genes into hematopoietic progenitors” Chapter 11, Gene Therapy of Cancer, Academic Press, San Diego, pp. 177-195. Movassagh, M. et al. (2000) “Retrovirus-mediated gene transfer into T cells: 95% transduction efficiency without further in vitro selection” Human Gene Therapy 11:1189-1200. Onodera, M. et al. (1998) “Successful peripheral T-lymphocyte-directed gene transfer for a subject with severe combined immune deficiency caused by adenosine deaminase deficiency” Blood 91:30-36. St. Croix, B., et al. (2000) “Genes expressed in human tumor endothelium” Science 289:1197-1202. Unutmaz, D. et al. (1999) “Cytokine signals are sufficient for HIV-1 infection of resting human T lymphocytes” J. Exp. Med. 111:1735-1746. Zennou, V., et al. (2000) “HIV-1 genome Nuclear import is mediated by a central DNA flap” Cell 101:173-185.

“Transfection”, which generally refers to techniques for the introduction of genetic material into a cell, has contributed greatly to the molecular and recombinant revolutions in biology. Examples of transfection techniques for use with higher eukaryotic cells include calcium phosphate precipitation, DEAE-dextran treatment, electroporation, microinjection, lipofectin, viral infection, and other methods found in numerous scientific textbooks and journals.

Among transfection techniques, the use of viral infection is unique in that a virus' naturally occurring means of introducing its genetic material into a cell is taken advantage of to transfer a nucleic acid molecule of interest into a cell. Examples of viruses modified and applied to such techniques include adenoviruses, adeno-associated viruses, herpes simplex viruses, and retroviruses. Generally, nucleic acid molecules of interest may be cloned into a viral genome. Upon replication and packaging of the viral genome, the resultant viral particle is capable of delivering the nucleic acid of interest into a cell via the viral entry mechanism.

Commonly, the viral genome is first made replication deficient by nucleic acid manipulation before the addition of the nucleic acid of interest. The resultant viral genome, or viral vector, requires the use of a helper virus or a packaging system to complete viral particle assembly and release from a cell. When a viral vector or viral particle is used to transfer genetic material of interest into a cell, the technique is referred to as “transduction”. Thus generally, to “transduce” a cell is to use a viral vector or viral particle to transfer genetic material into a cell.

Among transduction techniques, the use of retroviruses has been the subject of great interest for the genetic modification of mammalian cells. Of particular interest is the use of modified retroviruses to introduce genetic material into cells to treat genetic defects and other diseases. An example of this approach is seen in the case of cells of the hematopoietic system, where retroviruses and lentiviral vectors are the subject of intense research.

Movassagh, et al., for example, discuss their studies on their attempts to increase the efficiency of retrovirus mediated transduction by including results from studies on the cell cycle of activated T cells. As such, their results are dependent upon active cell division during transduction. The work is also limited to the use of a murine onco-retrovirus and the requirement for significant prestimulation of the cells before transduction.

June et al. (WO 96/34970) describe the use of T cell stimulation as a means to increase T cell transfection. Other work on T cell transduction with activated or stimulated cells include those of Douglas et al., Hooijberg et al, Onodera et al., Klebba et al., Barry et al., and Unutmaz et al. Unfortunately, none of this work demonstrated transduction efficiencies of greater than about 65%.

Costello et al. describe the transduction of both stimulated and non-stimulated T cells using Human Immunodeficiency Virus-1 (HIV-1) lentiviral vectors. They observed only about a maximum of 17% efficiency with stimulated primary T cells and less than 19% efficiency with non-stimulated T cells. They also noted a limited ability to increase efficiency to no more than 36% in stimulated T cells by including the presence of HIV-1 accessory proteins.

Chinnasamy et al. also describe an increase in the efficiency of transduction in the presence of HIV-1 accessory proteins in both non-stimulated and mitogen stimulated T cells. Like Movassagh et al, Chinnasamy et al. prestimulated blood lymphocytes for significant periods prior to transduction with a lentiviral vector. While Chinnasamy et al. initially observed a greater than 96% transduction efficiency three days after transduction, the percentage of stably transduced cells decreased to 71.2% two weeks after transduction. Haas et al. also observed transient transduction and “pseudotransduction” in cells transduced with a lentiviral vector capable of expressing a marker gene (green fluorescent protein). Even three days post transduction, significant (over 10%) transient transduction was detected based on non-integrative expression of the marker gene in transduced primary CD34+ cord blood cells. Such expression from transient transduction remained detectable at about 5% even seven days post-transduction. Only after about 10 days post transduction did expression from transient transduction mirror that in cells transduced with a markerless vector.

Therefore, Chinnasamy et al were not able to achieve stable transduction, where an integrated form of the viral vector has been inserted into the chromosomal DNA of the transduced cell, of primary lymphocytes beyond 71.2% as reflected by the efficiency after two weeks. This was despite the use of cytokines to prestimulated the cells. Furthermore, Chinnasamy describe their inability to significantly transduce (only 3.6% 14 days post transduction) non-stimulated lymphocytes with a HIV vector that did not express accessory proteins (Vif, Vpr, Vpu and Nef), even though the cells were later stimulated with the PHA mitogen and the IL-2 cytokine post-transduction. While the results were improved somewhat with the use of non-stimulated cells and vectors containing accessory proteins, in no case was the efficiency of stable transduction of stimulated or non-stimulated cells greater than 75% on day 14 post transduction, irrespective of the stimulatory protocol used with the vector.

Low frequencies of stable transduction with lantiviral vectors was also observed by Hass et al., who could only achieve a maximum stable transduction efficiency of less than 25%, seven days post transduction, with primary CD34 positive cord blood cells. Strikingly, this 25% upper limit of transduction could not be improved even after extremely high multiplicities of infection or vector concentrations, such as a multiplicity of infection (MOI) of up to 9000 and vector concentrations of up to 10⁸ infectious units per milliliter.

Follenzi et al. also used a very high MOI of 500 to transduce cells in the presence of a three cytokine cocktail containing interleukin-3 (IL-3), interleukin-6 (IL-6) and stem cell factor (SCF). Interestingly, use of the cocktail would render the cells unsuitable for human clinical transplantation.

Thus there remains a need to provide a more efficient means of stably transducing cells with vectors at high frequency. Additionally, there is a need for a more efficient means to transduce non-stimulated cells for use both as research tools and as a therapeutic agent.

In addition, there remains a need to transduce and culture and/or grow a large number of primary cells at the same time that can be used for laboratory and/or clinical applications. For example, it would be desirable to obtain one “batch” of cells from a transduction and not have to combine multiple batches of cells with varying transduction efficiencies and administer them to a subject.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

All references, publications, patent applications and patents cited herein are hereby incorporated by reference in their entireties, whether specifically incorporated or not.

### DISCLOSURE OF THE INVENTION

One aspect of the invention is a method for stable transduction of primary cells of the hematopoietic system and/or hematopoietic stem cells, comprising contacting, in vitro or ex vivo, the surface of the cells with both a lentiviral vector and at least one molecule which binds the cell surface, and culturing the cells in a ventilated vessel comprising two or more layers under conditions conducive to growth and/or proliferation, wherein the vessel is suitable for culturing at least about 100 million cells.

One aspect of the invention is a method for stable transduction of primary cells of the hematopoietic system and/or hematopoietic stem cells, comprising contacting, in vitro or ex vivo, the surface of the cells with both a lentiviral vector and at least one molecule which binds the cell surface, and culturing the cells in a ventilated vessel comprising two or more layers under conditions conducive to growth and/or proliferation, wherein the vessel is suitable for culturing at least about 100 million cells, and wherein the cells are transduced with the lentiviral vector at a multiplicity of infection (MOI) such that the copies of lentiviral vector per transduced cell is from about 0.5 to about 10; and wherein contacting the cells with a lentiviral vector is for about 24 hours and is optionally repeated at least once.

One aspect of the invention is a method for stable transduction of primary cells of the hematopoietic system and/or hematopoietic stem cells, comprising contacting, in vitro or ex vivo, the surface of the cells with both a lentiviral vector and at least one molecule which binds the cell surface, and culturing the cells in a ventilated vessel comprising two or more layers under conditions conducive to growth and/or proliferation, wherein the vessel is suitable for culturing at least about 100 million cells; and wherein at least about 50% of the cells are stably transduced after about seven to ten days, or at about 14 days; and optionally at least 50% of the cells remain stably transduced after about 14 days; or wherein at least about 75% of the cells are stably transduced after about seven to ten days, or at about 14 days, and optionally at least 75% of the cells remain stably transduced after about 14 days; or wherein greater than 80%, 85%, 89%, 90%, 91%, 92%, 93%, 94% or 95% of the cells are stably transduced after about 14 days; or wherein the cells are transduced with the lentiviral vector at a multiplicity of infection (MOI) of from about 2 to about 50, or from about 10 to about 30, or from at 10, or at about 20, or at about 30, or at about 40, or at about 50, or from about 1 to about 400, or less than 500; or wherein the cells are transduced with the lentiviral vector at a multiplicity of infection (MOI) such that the copies of lentiviral vector per transduced cell is from about 1 to about 100; or wherein the cells are transduced with the lentiviral vector at a multiplicity of infection (MOI) such that the copies of lentiviral vector per transduced cell is from about 0.5 to about 10; or wherein contacting the cells with a lentiviral vector is for about 24 hours and is optionally repeated at least once; and wherein the cell surface molecule does not induce apoptosis and the cell surface binding molecule results in the cell being more receptive to transduction by a viral lentiviral vector.

The primary cells can be isolated or derived from a subject. The primary cells can be isolated by one or more of the following procedures: (a) by apheresis of a subject's blood; or (b) from bone marrow from a subject's bone; or (c) by apheresis of an allogeneic subject's blood; or (d) from bone marrow from an allogeneic subject's bone. The primary cells can also be isolated using alternative techniques known to one of skill in the art.

Apheresis is a medical technology in which the blood of a donor or subject is passed through an apparatus that separates out one particular constituent and returns the remainder to the circulation.

Depending on the substance that is being removed, different processes are employed in apheresis. For example, if separation by weight is required, centrifugation would be the method of choice. Other exemplary methods that can be used in the invention involve absorption onto beads coated with an absorbent material.

There are numerous types of apheresis. For example: plasmapheresis—blood plasma; plateletpheresis (thrombapheresis, thrombocytapheresis)—blood platelets; leukapheresis—leukocytes (white blood cells); stem cell harvesting—circulating bone marrow cells are harvested to use in bone marrow transplantation; and LDL apheresis—removal of low density lipoprotein in subjects with familial hypercholesterolemia. These types of apheresis are merely exemplary. Other types may be known to one of skill in the art.

Blood components can be separated, for example, from a collected bag of whole blood or from a donor's blood flow before collected to a blood bag. Various types of blood components can be obtained by apheresis from donors. This includes, for example, platelets and blood plasma.

The various apheresis techniques may be used, for example, whenever the removed constituent is causing severe symptoms of disease. Generally, apheresis has to be performed fairly often, and is an invasive process. It is therefore usually only employed if other means to control a particular disease have failed, or the symptoms are of such a nature that waiting for medication to become effective would cause suffering or risk of complications.

Bone marrow can be obtained, stored, and manipulated by methods known to one of skill in the art. For example: U.S. Pat. No. 6,991,787 describes methods for obtaining bone marrow stromal cells; U.S. Pat. No. 6,110,176 describes methods to collect and store genetically compatible bone marrow; and U.S. Pat. No. 4,366,822 describes methods and apparatuses for bone marrow cell separation and analysis.

The subject can be infected with a human immunodeficiency virus (HIV), wherein optionally the HIV is HIV-1 or HIV-2. The subject can have cancer, wherein optionally the cancer is breast cancer. The subject can be either a human or an animal.

The primary cells can be enriched prior to contact with the lentiviral vector or cell surface binding molecule by passing the cells over a gradient density buffer and/or by immuno-purification over a magnetic field (magnetic cell sorting). The primary cells can also be enriched prior to or during the culturing and/or growth of the cells. Other know means of enriching a cell population, as known by one of skill in the art, can also be employed in the method of the invention. See, for example, U.S. Pat. No. 6,974,675 which describes processes for identifying and enriching cell-specific target structures. For example, the addition of a cytokine to tissue culture media can result in the enrichment of a specific cell type. Specifically, certain cell populations will die in the presence of certain cytokines in the media.

The contacting of the primary cells: (a) with the lentiviral vector occurs before contacting the cells with at least one cell surface binding molecule; or (b) with the lentiviral vector occurs simultaneously with contacting the cells with at least one cell surface binding molecule; or (c) with the lentiviral vector occurs after contacting the cells with at least one cell surface binding molecule; or (d) with the lentiviral vector occurs more than once; or (e) with the lentiviral vector occurs continuously, after the simultaneous contacting of the cells with the lentiviral vector and the at least one cell surface binding molecule; or (f) with cell surface binding molecule occurs continuously, after the simultaneous contacting of the cells with the lentiviral vector and the at least one cell surface binding molecule; or (g) with the lentiviral vector and the at least one cell surface binding molecule occurs continuously, after the initial simultaneous contact of the cells with the lentivirus vector and the at least one cell surface binding molecule; or (h) wherein any of (a) through (g) occurs at least once over a time period of about 24-36 hours.

The primary cells can be pre-stimulated with at least one cell surface binding molecule, and optionally the cells are pre-stimulated with the at least one cell surface binding molecule within about twenty four (24) hours prior to simultaneously contacting the cells with the lentiviral vector and the at least one cell surface binding molecule, or and optionally the cells are pre-stimulated with the at least one cell surface binding molecule within about 12 to 96 hours prior to simultaneously contacting the cells with the lentiviral vector and the at least one cell surface binding molecule.

The lentiviral vector can comprises at least one cis-acting nucleotide sequence derived from the gag, pol, env, vif, vpr, vpu, tat or rev genes, and optionally, wherein the sequence is not expressed or is a fragment or a mutant of the gag, pol, env, vif, vpr, vpu, tat or rev genes.

The lentiviral vector can be: (a) pseudotyped and optionally wherein the pseudotyped vector contains the vesicular stomatitis virus G envelope protein; or (b) pseudotyped, and wherein the pseudotyping comprises co-transfecting or co-infecting a packaging cell with both the lentiviral vector genetic material and genetic material encoding at least one envelope protein of another virus or a cell surface molecule; or (c) pseudotyped with a Rhabdovirus, and optionally wherein the Rhabdovirus is a Vesicular Stomatitis Virus envelope G (VSV-G) protein.

The primary cell can be a lymphocyte, a precursor of a lymphocyte, a CD4 positive cell, a hematopoietic stem cell of a CD4 positive cell, a CD8 positive cell, a hematopoietic stem cell of a CD8 positive cell, a CD34 positive cell, a hematopoietic stem cell of a CD34 positive cell, a dendritic cell, a cell capable of differentiating into a dendritic cell, a human primary cell of the hematopoietic system and/or a human hematopoietic stem cell, a precursor of a human hematopoietic stem cell, an astrocyte, a skin fibroblast, a epithelial cell, a neuron, a dendritic cell, a leukocyte, a cell associated with the immune response, a vascular endothelial cell, a tumor cell, a tumor vascular endothelial cell, a liver cell, a lung cell, a bone marrow cell, an antigen presenting cell, a stromal cell, an adipocyte, a muscle cell, a pancreatic cell, a kidney cell, an ovum, a spermatocyte, a cell that contributes to the germ line, an embryonic pluripotential stem cell or a progenitor cell, a blood cell, a non-nucleated cell, a platelet cell, or an erythrocyte, or a derivative thereof.

The at least one cell surface binding molecule: (a) comprises a polypeptide, a lipid, a nucleic acid, a carbohydrate or an ion; or (b) comprises an antibody, an antigen binding fragment, a ligand, or a cell surface molecule; or (c) comprises FLT-3 ligand, TPO ligand, or Kit ligand, or a polypeptide or other binding molecule that is a cell surface binding analog of FLT-3 ligand, TPO ligand, or Kit ligand; or (d) comprises CD34, CD3 ligand, CD28 ligand, CD25 ligand, CD71 ligand, or CD69 ligand, or a polypeptide or other binding molecule that has the same cell surface binding specificity of CD34, CD3, CD25, CD28, CD69 or CD71 ligand; or (e) comprises a composition comprising GM-CSF, IL-4, and TNF-alpha; GM-CSF and interferon-alpha; or a polypeptide or other binding molecule that is a cell surface binding analog of GM-CSF, IL-4, and TNF-alpha; GM-CSF or interferon-alpha; or (f) comprises a CD3 antibody or cell surface binding fragment thereof, a CD28 antibody or cell surface binding fragment thereof, a combination of the antibody and cell surface binding fragment thereof, and a binding molecule that has the same cell surface binding specificities as the antibody; or (g) comprises a combination of CD3 and CD28 antibodies immobilized on a bead or a surface, wherein optionally the bead or surface comprises coated beads; or (h) comprises two or more cell surface binding molecules selected from any of (a) through (g); or (i) comprises another molecule that is used to increase or reinforce the ability of the molecule to bind to the surface of the cell; or (j) is complexed with another molecule; or (k) is found on the primary cell's surface and binds to the surface of another cell.

The cell culture conditions can comprise: (a) further incubation with a cell surface binding molecule or a cytokine; or (b) further incubation with interleukin-2; or (c) culturing the cells for about seven days; or (d) culturing the cells for about 14 days.

The lentiviral vector can be: (a) derived from a human immunodeficiency virus (HIV); or (b) derived from HIV-1, HIV-2, or a combination thereof; or (c) a chimeric vector comprising HIV sequences, wherein optionally the HIV sequences comprise HIV-1 and HIV-2 sequences; or (d) VRX496, or a derivative of VRX496.

The contacting of the cells with the lentiviral vector or the cell surface binding molecule can occur ex vivo in a mixed or pure cell culture, a tissue or an organ system.

Another aspect of the invention is a method to introduce a genetic material into a cell comprising ex vivo introduction of the cell transduced by any of the methods described herein into a living subject, a tissue, an organ, a blastocyst or an embryonic stem cell.

Another aspect of the invention is the use of a primary cell of the hematopoietic system or hematopoietic stem cell transduced by any of the methods described herein for the preparation of a pharmaceutical composition. The pharmaceutical composition can be used for the treatment or prevention of a viral infection in a subject, for the treatment or prevention of an HIV infection in a subject, or for the treatment or prevention of cancer. The cancer can be any type of cancer, for example breast cancer, or any cancer of the endothelial cells.

Another aspect of the invention is a pharmaceutical composition for gene therapy to treat or prevent an abnormality caused by a genetic defect, or to treat, diagnose, alleviate or prevent a tumor or a cancer, produced by any of the methods described herein, and optionally wherein the abnormality caused by a genetic defect or tumor or cancer is a breast cancer tumor.

Another aspect of the invention is a pharmaceutical composition for gene therapy to treat or prevent an abnormality caused by an infection, produced by any of the methods described herein. The infection can be a viral infection, and optionally wherein the viral infection is a human immunodeficiency virus (HIV) infection. The pharmaceutical composition is formulated for use ex vivo.

Another aspect of the invention is a method for stable transduction of primary cells of the hematopoietic system and/or hematopoietic stem cells, comprising contacting, in vitro or ex vivo, the surface of the cells with both a lentiviral vector and at least one molecule which binds the cell surface, and culturing the cells in a ventilated vessel comprising two or more layers under conditions conducive to growth and/or proliferation, wherein the vessel is suitable for culturing at least about 100 million cells, and wherein the contacting of the primary cells with the cell surface molecule makes the cells more receptive to transduction by the lentiviral vector.

The presence of the cell surface molecule on the surface of the primary cells can result in: (a) the cell's chromatin being more receptive to DNA integration; or (b) integration of the lentiviral vector into a cellular site favorable for expression of a gene from the lentiviral vector; or (c) more efficient entry of a nucleic acid containing capsid into the cytoplasm of the cells; or (d) more efficient entry of the virus across a cell membrane or across an internal membranous structure of the cells; or (e) the primary cells being more permissive for nuclear import of the genetic material contained in the viral vector.

The cell surface binding molecule, antibody, antigen binding fragment, ligand or cell surface molecule comprises: anti-CD3 or anti-CD28 antibodies which bind the cells and make them more receptive to vector transduction; antibodies or ligands for the FLT-3 ligand, TPO, and Kit ligand receptors, which bind the cells and make them more receptive to vector transduction; antibodies or ligands for GM-CSF and IL-4 receptors, which bind dendritic cells or their precursors, monocytes, CD34 positive stem cells, or their differentiated progenitor cells on the dendritic cell lineage, and make them more receptive to vector transduction; a polypeptide, nucleic acid, carbohydrate, lipid or ion, or a polypeptide, nucleic acid, carbohydrate, lipid or ion complexed with another substance that binds CD1a, CD1b, CD1c, CD1d, CD2, CD3γ, CD3δ, CDε, CD4, CD5, CD6, CD7, CD8α, CD8β, CD9, CD10, CD11a, CD11b, CD11c, CDw12, CD13, CD14, CD15, CD15s, CD16a, CD16b, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45R, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD67, CD68, CD69, CDw70, CD71, CD72, CD73, CD74, CDw75, CDw76, CD77, CD79cc, CD79(3, CD80, CD81, CD82, CD83, CD84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CDw109, CD114, CD115, CD116, CD117, CD118, CD119, CD120a; CD120b, CD121a, CD121b, CD122, CD123, CDw124, CD125, CD126, CDw127, CDw128a, CDw128b, CDw130, CDw131, CD132, CD133, CD134, CD135, CD136, CDw137, CD138, CD139, CD140a, CD140b, CD141, CD142, CD143, CD144, CDw145, CD146, CD147, CD148, CDw149, CD150, CD151, CD152, CD153, CD154, CD155, CD156, CD157, CD158a, CD158b, CD161, CD162, CD163, CD164, CD165, CD166 or TCRζ on the cells and makes them more receptive to vector transduction.

Another aspect of the invention is a method for stable transduction of a primary cell of the hematopoietic system and/or a hematopoietic stem cell isolated from an HIV-infected subject, comprising the steps of: (a) isolating from the HIV-infected subject primary cells of the hematopoietic system cells or hematopoietic stem cells; (b) optionally, pre-stimulating the primary cells or hematopoietic stem cells with at least one cell surface binding molecule; (c) contacting simultaneously in vitro or ex vivo the hematopoietic system cells or hematopoietic stem cells with a lentiviral vector and at least one cell surface binding molecule; and (d) culturing the cells in a ventilated vessel comprising two or more layers under conditions conducive to growth and/or proliferation, wherein the vessel is suitable for culturing at least about 100 million cells.

Another aspect of the invention is a system comprising: (a) a ventilated vessel comprising two or more layers; and (b) isolated non-adherent primary cells of the hematopoietic system and/or hematopoietic stem cells. The primary cells of the system can be any of the cells as described above for the methods of the invention.

The multilayer vessel can be any shape that is practical to culture and/or grow cells in. For example, the vessel can be rectangular in shape, square in shape, or rectangular in shape with a curved edge, or square in shape with a curved edge.

Tissue culture flasks or vessels are widely used in the laboratory to grow cells. Typically, these flasks are used to culture cells in a culture medium wherein the cells are adhered to an interior surface of the flask. The cells are introduced into the flask or vessel through an opening. The flask or vessel is closed, though allowing for ventilation, and inserted into a stacking facility or chamber, such as an oven, to facilitate the growth of the cells in the medium.

Primary cells of the hematopoietic system and/or hematopoietic stem cells are grown in culture bags, or other single layer vessels or flasks. This limits the number of cells that can be grown at one time. Multilayer flasks or vessels with flat surfaces allow a large number of cells to be cultured at one time but have been used for cells that adhere to the flat surfaces. Primary cells of the hematopoietic system and/or hematopoietic stem cells are non-adherent cells. However, these cells can be grown in multilayer flasks or vessels allowing a larger number of cells to be grown at one time. One example of a multilayer vessel is a cell factory.

The methods and compositions of the invention allow for large scale culturing and/or growth of non-adherent primary cells of the hematopoietic system and/or hematopoietic stem cells in a multilayer tissue culture flask or vessel, resulting in the growth of about at least 100 million cells.

Cell factories, or variations thereof of the concept of a cell factory can be used for large scale production of, for example, vaccines, monoclonal antibodies or pharmaceuticals. They are ideal for adherent cells, but can also be used for suspension culture. The growth kinetics of the cells remain unaltered from laboratory scale culture. Cell factories, are available, for example, in 1, 2, 4, 10 and 40 tray versions for easy scale-up. They have a low contamination risk and compact design.

The following descriptions are examples of the types of vessels that can be used in the methods of the invention. They are merely exemplary and are not meant to limit the scope of the invention.

Examples of types of vessels in which cells can be grown are shown in FIG. 1. The vessel can be any shape that is practical to grow cells in, for example, square, rectangle, circular, oval, or square or rectangular with shaped ends. The vessel must be more than one layer, but the upper number of how many layers the vessel can comprise is only limited by the size of the chamber, oven, or container that the cells are cultured and/or grown in. The vessel can be made of plastic, for example, or any other material that would be suitable for culturing and/or growing cells in.

A cell factory is a stack of chambers sealed together into a single unit, sharing common vent and fill ports. Each chamber has, for example, a flat growth surface of 632 cm². Cell factories are used for large scale cell culture and production of bio-materials such as vaccines, monoclonal antibodies and interferon. Cell factories provide a large amount of growth surface in a small area with easy handling and low risk of contamination. A 40-chamber unit with a growth area of 25,280 cm corresponds to 14 large roller bottles (1,750 cm each). Only one filling and emptying operation is required with the cell factory, compared to 14 with the roller bottles. Cell factories are sterile. Cell factories provide a large growth surface in limited space areas.

Other aspects of a cell factory are, for example, a media reservoir consisting of an aspirator bottle with vented stopper that will hold the cell suspension. Installing, culturing and harvesting cells with a cell factory is known in the art. Variations on the types of incubators and cell culturing devices that can be used in the invention are described, for example, in published patent applications 20060057713 and 20060057712, and U.S. Pat. No. 6,114,165.

Due to the height of the 10 and 40-chamber cell factories, cells cannot be viewed with common microscopes. Most often, cells are seeded in a 1-chamber or 2-chamber cell factory as a control. Cells grown in each of these products can be viewed under a microscope. Inverted stereo microscopes with a powerful light source on the view side, and adjusted for the height of the cell factory have been used to view the first few layers.

Dimensions and Culture Areas of Exemplary Cell Factories:

| Description | Dimensions L × W × H (mm) | Culture Area (cm²) |
|---|---|---|
| 1-Chamber CF | 335 × 205 × 37 | 632 |
| 2-Chamber CF | 335 × 205 × 52 | 1,264 |
| 10-Chamber CF | 335 × 205 × 190 | 6,320 |
| 10-Chamber CF | 335 × 205 × 190 | 6,320 |
| 40-Chamber CF | 335 × 205 × 700 | 25,284 |

The methods of the present invention produce a novel composition—a sample, “population” or “batch” of cells having a high transduction level and that comprise at least about 100 million cells.

The present invention provides highly efficient methods, and compositions related thereto, for the stable transduction of cells with viral vectors and viral particles. By “stable transduction,” it is meant where an integrated form of the viral vector has been inserted into the chromosomal DNA of the transduced cell. The methods comprise exposing the cells to be transduced to contact with at least one molecule that binds the cell surface. This contacting step may occur prior to, during, or after the cells are exposed to the viral vector or viral particle. Hereinafter, the term “viral vector” will be used to denote any form of a nucleic acid derived from a virus and used to transfer genetic material into a cell via transduction. The term encompasses viral vector nucleic acids, such as DNA and RNA, encapsulated forms of these nucleic acids, and viral particles in which the viral vector nucleic acids have been packaged.

The present invention also includes the use of the transduced cells in other applications, including production of useful gene products and proteins by expression of a nucleic acid present in the vector or therapy of living subjects afflicted, or at risk of being afflicted with a disease. For example, the subject is human.

The at least one molecule that binds the surface of the cells to be transduced includes any molecule that physically interacts with a receptor, marker, or other recognizable moiety on the surface of the cells. In principle, any cell surface binding molecule may be used for high efficiency transduction of cells. Without binding the invention to theory, the cell surface binding molecules may result in the host cell's chromatin being more receptive to DNA integration; in preferential integration of a viral vector into a site favorable for vector gene expression; in more efficient entry of the nucleic acid containing capsid into the cytoplasm; in more efficient entry of the virus across the cell membrane or internal membranous structures such as the endosome; or in making the cell more permissive for nuclear import of the viral vector's genetic material. The methods of the invention may also involve more than one of the above possibilities. Also, and as evident from the number and diversity of the above possibilities, the invention cannot be limited to any one theory. Instead, and given the extraordinary discovery of the invention in the stable transduction of up to 100% of the treated cells without negatively affecting the possible use of the cells in human therapy, the invention should be viewed as opening a new approach in the field of human cell therapy.

Not all cell surface binding molecules, however, will result in the efficient and stable transduction by viral vectors. For example, binding to a cell surface molecule that induces apoptosis will not result in efficient transduction of the cell, but rather cell death. Although cell death may be preferred for the killing of cells (e.g., tumor cells) it is not preferred for the stable transduction of cells with vectors containing payload genes or nucleic acid sequences. A preferred cell surface binding molecule results in the cell being more receptive to transduction by a viral vector. Examples of such molecules include an antibody for a specific cell surface receptor or portion thereof as well as a ligand or binding domain for such a receptor. Moreover, antigen-binding fragments of antibodies, such as F_{ab} and Fᵥ fragments are contemplated for use in the present invention. The binding domain for the specific, cell-surface receptor can contain a single epitope or two or more epitopes.

Examples of cell surface binding molecules for use in the invention are anti-CD3 and anti-CD28 antibodies which bind T cells and make them more receptive to vector transduction. Other cell surface binding molecules are antibodies or ligands for the FLT-3 ligand, TPO, and Kit ligand receptors, which make cells expressing the receptors, such as hematopoietic stem cells, more receptive to vector transduction. Additional cell surface binding molecules are antibodies or ligands for GM-CSF and IL-4 receptors, which make dendritic cells or their precursors, such as monocytes, CD34 positive stem cells, or their differentiated progenitor cells on the dendritic cell lineage, more receptive to vector transduction. Other cell surface binding molecules include molecules found on cell surfaces which bind the surface of another cell.

Additional examples of cell surface binding molecules include polypeptides, nucleic acids, carbohydrates, lipids, and ions, all optionally complexed with other substances. The molecules can bind factors found on the surfaces of blood cells, such as CD1a, CD1b, CD1c, CD1d, CD2, CD3γ, CD3δ, CD3ε, CD4, CD5, CD6, CD7, CD8α, CD8β, CD9, CD10, CD11a, CD11b, CD11c, CDw12, CD13, CD14, CD15, CD15s, CD16a, CD16b, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45R, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD67, CD68, CD69, CDw70, CD71, CD72, CD73, CD74, CDw75, CDw76, CD77, CD79α, CD79β, CD80, CD81, CD82, CD83, CD84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CDw109, CD114, CD115, CD116, CD117, CD118, CD119, CD120a, CD120b, CD121a, CD121b, CD122, CD123, CDw124, CD125, CD126, CDw127, CDw128a, CDw128b, CDw130, CDw131, CD132, CD133, CD134, CD135, CD136, CDw137, CD138, CD139, CD140a, CD140b, CD141, CD142, CD143, CD144, CDw145, CD146, CD147, CD148, CDw149, CD150, CD151, CD152, CD153, CD154, CD155, CD156, CD157, CD158a, CD158b, CD161, CD162, CD163, CD164, CD165, CD166, and TCRζ. Small letters (e.g., “a” or “b”) indicate complex CD molecules composed of multiple gene products or belonging to families of structurally related proteins. The notation “w” refers to putative CD molecules that have not yet been fully confirmed.

Additional molecules that bind factors found on the surfaces of lymphocytes, T cells and leukocytes, are CD2, CD3γ, CD3δ, CD3ε, CD5, CD6, CD7, CD8α, CD8β, CD9, CD11a, CD18, CD25, CD26, CD27, CD28, CD29, CD30, CD37, CD38, CD39, CD43, CD44, CD45R, CD46, CD48, CD49a, CD49b, CD49c, CD49d, CD49e, CD49f, CD50, CD53, CD54, CD56, CD57, CD58, CD59, CDw60, CD62L, CD68, CD69, CDw70, CD71, CD73, CDw75, CDw76, CD84, CD85, CD86, CD87, CD89, CD90, CD94, CD96, CD97, CD98, CD99, CD100, CD101, CD103, CD107a, CD107b, CDw108, CDw109, CD118, CD119, CD120b, CD121a, CD122, CDw124, CDw127, CDw128a, CDw130, CD132, CD134, CDw137, CD140a, CD140b, CD143, CD146, CD148, CD152, CD153, CD154, CD155, CD161, CD162, CD165, CD166, and TCRζ.

Additional antibodies and molecules that bind to the surface of cells, and suitable for use in the present invention, are disclosed in Linscott's Directory of Immunological and Biological Reagents, 11th Edition, January 2000, Publisher: W. D. Linscott, Petaluma, CA, which is hereby incorporated by reference as if fully set forth. In some embodiments of the invention, however, the cell surface binding molecule is not a cytokine.

While the invention may be practiced by use of soluble cell surface binding molecules that promote vector transduction of cells, other embodiments include the use of immobilized cell surface binding molecules. For example, the immobilized molecules are antibodies. Alternatively, immobilization may be via use of other cells that express the cell surface binding molecules. An exemplary method for the efficient transduction of hematopoietic stem cells is to include bone marrow stromal cells, expressing ligands on their surface that facilitate stem cell maintenance without differentiation, during transduction. The stimulating cells are not restricted to native cells, but any cell can be engineered to express the appropriate cell surface binding molecule in order to provide the correct stimulus for transduction.

Additional molecules that increase or reinforce the ability of the at least one molecule to bind the cell surface may also be included. For example, a soluble form of a (primary) antibody for a specific cell surface receptor may be used in combination with a secondary antibody that can crosslink primary antibodies already bound to the cell surface.

Of course any cell can be used in the practice of the invention. For example, the cell to be transduced is a eukaryotic cell. For example, the cell is a primary cell. Cell lines, however, may also be transduced with the methods of the invention and, in many cases, more easily transduced. In one embodiment, the cell to be transduced is a primary lymphocyte (such as a T lymphocyte) or a macrophage (such as a monocytic macrophage), or is a precursor to either of these cells, such as a hematopoietic stem cell. Other exemplary cells for transduction in general are cells of the hematopoietic system, or, more generally, cells formed by hematopoiesis as well as the stem cells from which they form and cells associated with blood cell function. Such cells include granulocytes and lymphocytes formed by hematopoiesis as well as the progenitor pluripotent, lymphoid, and myeloid stem cells. Cells associated with blood cell function include cells that aid in the functioning of immune system cells, such as antigen presenting cells like dendritic cells, endothelial cells, monocytes, and Langerhans cells. In one embodiment, the cells are T lymphocytes (or T cells), such as those expressing CD4 and CD8 markers.

In another embodiment, the cell is a primary CD4+ T lymphocyte or a primary CD34+ hematopoietic stem cell. However, and given that the viral vectors for use in the invention may be pseudotyped with Vesicular Stomatitis Virus envelope G protein (as discussed below), any cell can be transduced via the methods of the present invention. Such a cell includes, but is not limited to, an astrocyte, a skin fibroblast, a epithelial cell, a neuron, a dendritic cell, a lymphocyte, a cell associated with the immune response, a vascular endothelial cell, a tumor cell, a tumor vascular endothelial cell, a liver cell, a lung cell, a bone marrow cell, an antigen presenting cell, a stromal cell, an adipocyte, a muscle cell, a pancreatic cell, a kidney cell, an ovum or spermatocyte (e.g., to create transgenic animals), a cell that contributes to the germ line, a embryonic pluripotential stem cell or its progenitors, a blood cell including non-nucleated cells such as platelets and erythrocytes, and the like. For example, the cell is of a eukaryotic, multicellular species (e.g., as opposed to a unicellular yeast cell), or, is of mammalian origin, e.g., a human cell.

A cell to be transduced can be present as a single entity, or can be part of a larger collection of cells. Such a “larger collection of cells” can comprise, for instance, a cell culture (either mixed or pure), a tissue (e.g., epithelial, stromal or other tissue), an organ (e.g., heart, lung, liver, gallbladder, urinary bladder, eye, and other organs), an organ system (e.g., circulatory system, respiratory system, gastrointestinal system, urinary system, nervous system, integumentary system or other organ system), a blastocyst, a embryonic stem cell a cell from a fetus (e.g., for the treatment of a genetic disorder/disease or for creating transgenic animals), diseased tissues such as a tumor or the site of an infection, or an organism (e.g., a bird, mammal, marine organism, fish, plant or the like). The organs/tissues/cells being targeted can be of the circulatory system (including for example, but not limited to heart, blood vessels, and blood), respiratory system (e.g., nose, pharynx, larynx, trachea, bronchi, bronchioles, lungs, and the like), gastrointestinal system (including for example mouth and oral tissues, pharynx, esophagus, stomach, intestines, salivary glands, pancreas, liver, gallbladder, and the like), mammary system (such as breast epithelial cells and supporting cells in the tissue), urinary system (such as kidneys, uterus, urinary bladder, urethra, and the like), nervous system (including, but not limited to, brain and spinal cord, and special sense organs, such as the eye) and integumentary system (e.g., skin).

The cells to be transduced can be selected from the group consisting of heart, blood vessel, including tumor blood vessels and blood vessels associated with infected or diseased tissue, bone marrow, blood, brain, lymphatic tissue, lymph node, spleen, lung, liver, gallbladder, urinary bladder, and eye cells. In one particular embodiment of the invention, the cell is autologous to the intended host for use, but cells allogenic, partially mismatched, completely mismatched, or even xenogenic to the host may also be used. Furthermore, universal donor cells, suitable for use in any given host organism, a related group of organisms or a species, such as human beings, may be transduced. This latter embodiment of the invention is particularly important in the transplantation of cells, tissues or organs, where the source of the transduced cells may be critical to the outcome of the transplant.

Another type of cell for transduction by the methods of the invention is a tumor cell, a diseased cell, or a cell at risk for becoming abnormal over time due to its genetic makeup or the genetic makeup of other cells present in the same organism. The latter embodiment permits the transduced cells of the invention to be used in prophylaxis. Breast cancer is one example of a disease process where prognostic indicators would allow for treatment with the transduced cells of the invention as early genetic intervention before the disease ensues. However, the methods of the invention may also be used in the therapeutic treatment of breast cancer after the disease has been detected. Additional applications of the invention in cancer therapy are numerous, and one skilled in the art would be able to use the invention set out herein for the treatment of many types of cancers without undue experimentation.

By way of example, and without limiting the present invention, one application is in breast cancers that are estrogen dependent. The cancer cells in estrogen-dependent breast cancer would be, for example, transduced by using antibodies or ligands that bind the estrogen receptor in combination with a therapeutic viral vector. The vector may contain, for example, a tumor inhibiting gene, such as the Herpes virus thymidine kinase gene. The transduced cells can thus be selectively killed by the addition of gancyclovir, a pro-drug that can be activated by Herpes thymidine kinase. Additional examples of tumor inhibiting genes and a corresponding pro-drug are numerous and well known in the art and may be selected by the skilled artisan without undue experimentation. The use of activatable pro-drugs in combination with application of the transduction methods of the invention may be broadly applied to other tumor types, and the above example does not limit the invention to tumors that are hormonal dependent or dependent upon some soluble factor for growth or proliferation.

For example, Her-2/neu positive tumor cells are not estrogen dependent, and a poor prognostic indicator since non-estrogen dependent tumors containing such cells are highly resistant to treatment with drugs such as taxol, an estrogen antagonist. Another embodiment to the invention is to include antibodies or other molecules that bind Her-2/neu or heregulin with viral vector preparations during transduction of tumor contaminated cells, such as in a bone marrow transplantation protocol. Alternatively, the transduction may be made directly to the tumor site, or intra vascularly in vivo with vectors that would modulate tumorogenesis.

Yet another embodiment of the invention is to target the tumor vasculature, alone or in combination with targeting the tumor cells. St Croix et al., which is hereby incorporated by reference as if fully set forth, have identified genes that are specifically overexpressed in tumor endothelial cells as compared to normal endothelium by SAGE analysis. Many of these genes encode cell surface molecules, such as the Thy-1 cell surface antigen or Endo180 lectin. All of the upregulated cell surface factors may be bound by a cell surface binding molecule of the invention to provide a stimulus for efficient stable gene transduction. Thus, an approach for tumor therapy would be to destroy the tumor vasculature by killing tumor endothelial cells after transducing them with a therapeutic viral vector in the presence of cell surface binding molecules that bind selectively to tumor vasculature and not normal endothelial cells.

In yet another embodiment of the invention is selective expression of an anti-tumor gene in tumor vasculature by incorporating elements (e.g., promoters or cis-acting stabilizing/degradation elements on mRNA) in the viral vector that selectively promote expression of the anti-tumor gene in tumor but not in normal vascular endothelium. Such methods can occur ex vivo, in vitro or in vivo. In vivo is a one method for therapy if the tumor vascular endothelium is targeted. Alternatively, and if the goal is to purge bone marrow of contaminating tumor cells for bone marrow transplantation, for example, then the method for therapy can occur ex vivo or in vitro.

Furthermore, in vivo uses are not restricted to disease states and can be used to transduce normal cells. For example, the invention may be used to transduce hematopoietic stem cells in vivo in the bone marrow. Any combination of antibodies or other cell surface binding molecules, such as FLT-3 ligand, TPO and Kit ligand, or functional analogs thereof, or stromal cells expressing the cell surface binding molecule, could be added with vector upon direct injection into the bone marrow for high efficiency bone marrow transduction. The term “functional analog” refers to any molecule that retains the cell surface binding activity of a cell surface binding molecule of the invention. Such functional analogs include fragments of FLT-3 ligand, TPO and Kit ligand; FLT-3 ligand, TPO and Kit ligand molecules containing one or more amino acid substitutions, additions or deletions; and antibodies that mimic the cell surface binding activity of a cell surface binding molecule.

An alternative approach to the above is to use bone marrow stromal cells as producer cells for the viral vector and thus provide the vector and cell surface binding molecule via cell therapy and not as a vector preparation. Another example is the transduction of T cells or dendritic cells by adding functional analogs of CD3 and CD28 antibodies or GM-CSF and IL-4, respectively, with vector during subcutaneous injection. The lymph in the subcutaneous tissue would drain the vector and stimulants into the lymph nodes for efficient transduction of the targeted cells.

The present invention includes the advantage that optionally, purification of the cell to be transduced is not essential. Transduction of mainly a cell type of interest can be accomplished by the choice of cell surface moiety to be bound. Thus in a mixed population of blood cells, for example, transduction of cells expressing CD3, such as certain T cells, will be enhanced when CD3 specific antibodies are used to interact with the cells. This will occur in preference over other cell types in the population, such as granulocytes and monocytes that do not express CD3.

The invention also encompasses the transduction of purified or isolated cell types if desired. The use of a purified or isolated cell type provides additional advantages such as higher efficiencies of transduction due to higher vector concentrations relative to the cell to be transduced.

When purified T cells are to be transduced, the at least one molecule binds a cell surface molecule found on T cells. Examples of such cell surface molecules include CD3, CD28, CD25, CD71, and CD69. Examples of molecules that bind to these cell surface molecules include antibodies and monoclonal antibodies that recognize them, many of which are commercially available or readily and routinely prepared using standard techniques without undue experimentation. In an exemplary embodiment for the transduction of CD4+ or CD8+ cells, monoclonal antibodies that recognize CD3 and/or CD28 may be used. Commercially available examples of such antibodies include OKT3 for CD3 and CD28.2 for CD28. These antibody molecules may be used in a soluble form, optionally later crosslinked by other molecules, or in an immobilized form such as on beads or other solid surfaces. In one embodiment of the invention, the antibodies are immobilized on the surface of the vessel, such as the walls of a tissue culture well, plate, or bag used for the viral vector mediated transduction. Without being bound by theory, use of immobilized antibodies on the surface where cells adhere or make contact may increase local concentrations of cell surface interactions on the cell surface.

When hematopoietic stem cells are to be transduced, antibodies specific for the hematopoietic stem cell receptor of the FLT-3 ligand, TPO (Thrombopoietin or Megakaryocyte Growth and Development Factor), or Kit ligand may be used as the cell surface binding molecule. Alternatively, antibodies to stem cell positive cell markers, including, but not limited to CD34 or AC133, may be used. When a ligand containing compound or composition is used as a cell surface binding molecule, the whole native ligand-containing proteins, ligands or ligands bound to heterologous proteins can be used either in a soluble or immobilized form. Immobilized forms include attachment to microbeads, directly or indirectly, using, for example, avidin/biotin.

Alternatively, the ligand may be expressed in the viral envelope of the viral vector, optionally in the form of a chimeric or fusion proteins, and/or complexed (covalently or non-covalently) with one or more other protein(s). In such embodiments, the cell surface binding molecule is presented in combination with the viral vector as a single composition for transducing cells. Additional examples of cell surface binding molecules that may be expressed in viral envelopes include the numerous surface factors listed above.

Other cell surface binding molecules, such as antibodies or fragments thereof, are those that bind to the hematopoietic stem cell receptors of Notch or Delta, or the Notch or Delta proteins themselves, or the ligands of Notch or Delta that are bound to heterologous proteins. Delta and Notch encode cell surface proteins that influence a wide variety of cell fate decisions in Drosophila development. Vertebrate homologues of Delta and Notch are essential for normal embryonic development. Delta homologues are importantly involved in the regulation of hematopoiesis. Delta-Serrate-lag2 (DSL), a soluble form of a homologue, enhances expansion of primitive hematopoietic precursors. When combined with hematopoietic growth factors, including interleukin-3 (IL-3), granulocyte colony-stimulating fact or (G-CSF) or granulocyte-macrophage colony-stimulating factor (GM-CSF), DSL promotes the expansion of primitive hematopoietic progenitors and at the same time inhibited the differentiation of primitive precursors into more mature precursor cells responsive to IL-3 alone (see Han et al.). DSL most likely acts by activating the Notch receptor expressed in hematopoietic cells, modulating cellular competence to respond to conventional hematopoietic growth factors by selectively blocking cell differentiation, but not proliferation signals (see Han and Moore, Blood 1999). Therefore, Delta and Notch homologues, antibodies that are functional analogs to the homologues, are examples of cell surface binding molecules for use in achieving greater than 75% efficient vector transduction of cells, particularly hematopoietic stem cells.

The present invention includes viral vectors, and compositions comprising them, for use in the disclosed methods. The vectors can be retroviral (family Retroviridae) vectors, for example, lentiviral vectors. Other retroviral vectors, such as oncoviral and murine retroviral vectors, may also be used. Additional vectors may be derived from other DNA viruses or viruses that can convert their genomes into DNA during some point of their life cycle. The viruses can be, for example, from the families Adenoviridae, Parvoviridae Hepandaviridae (including the hepatitis delta virus and the hepatitis E virus which is not normally classified in the Hepandaviridae), Papoviridae (including the polyomavirinae and the papillomavirinae), Herpesviridae, and Poxyiridae.

Additional viruses of the family Retroviridae (i.e., a retrovirus), are of the genus or subfamily Oncovirinae, Spumavirinae, Spumavirus, Lentivirinae, and Lentivirus. An RNA virus of the subfamily Oncovirinae is desirably a human T-lymphotropic virus type 1 or 2 (i.e., HTLV-1 or HTLV-2) or bovine leukemia virus (BLV), an avian leukosis-sarcoma virus (e.g., Rous sarcoma virus (RSV), avian myeloblastosis virus (AMV), avian erythroblastosis virus (AEV), and Rous-associated virus (RAV; RAV-0 to RAV-50), a mammalian C-type virus (e.g., Moloney murine leukemia virus (MuLV), Harvey murine sarcoma virus (HaMSV), Abelson murine leukemia virus (A-MuLV), AKR-MuLV, feline leukemia virus (FeLV), simian sarcoma virus, reticuloendotheliosis virus (REV), spleen necrosis virus (SNV)), a B-type virus (e.g., mouse mammary tumor virus (MMTV)), and a D-type virus (e.g., Mason-Pfizer monkey virus (MPMV) and “SAIDS” viruses).

An RNA virus of the subfamily Lentivirus is desirably a human immunodeficiency virus type 1 or 2 (i.e., HIV-1 or HIV-2, wherein HIV-1 was formerly called lymphadenopathy associated virus 3 (HTLV-III) and acquired immune deficiency syndrome (AIDS)-related virus (ARV)), or another virus related to HIV-1 or HIV-2 that has been identified and associated with AIDS or AIDS-like disease. The acronym “HIV” or terms “AIDS virus” or “human immunodeficiency virus” are used herein to refer to these HIV viruses, and HIV-related and -associated viruses, generically. Moreover, a RNA virus of the subfamily Lentivirus, can be, for example, a Visna/maedi virus (e.g., such as infect sheep), a feline immunodeficiency virus (FIV), a bovine lentivirus, a simian immunodeficiency virus (SIV), an equine infectious anemia virus (EIAV), or a caprine arthritis-encephalitis virus (CAEV), or a combination thereof.

An exemplary lentiviral vector is one derived from HIV, for example, HIV-1, HIV-2, or chimeric combinations thereof. Of course different serotypes of retroviruses, especially HIV, may be used singly or in any combination to prepare vectors for use in the present invention. Vectors of the invention can, for example, contains cis acting elements that are present in the wild-type virus, but not present in a “basic” lentiviral vector. A “basic” lentiviral vector contains minimally, LTRs and packaging sequences in the 5′ leader and gag encoding sequences, but can also optionally contain the RRE element to facilitate nuclear export of vector RNA in a Rev dependent manner. A vector can additionally comprise nucleotide sequences that enhance the efficiency of transduction into cells.

An example of such a vector is pN2cGFP, a vector that contains the complete sequences of gag and pol. Another example is a vector that contain sequences from about position 4551 to position 5096 in pol (reference positions from the pNL4-3 sequence, Accession number M19921, HIVNL43 9709 bp, kindly provided by C. E. Buckler, NIAID, NIH, Bethesda, Md.). However any cis-acting sequence from the wt-HIV that can improve vector transduction efficiency may be used. Other examples of vectors capable of efficient transduction via the present invention are cr2HIV constructs as described in U.S. Pat. No. 5,885,806.

A previously identified sequence that is insufficient to significantly increase transduction efficiency described by Zennou et al. (2000) as a central DNA flap (a 178 base pair fragment from positions 4793 to 4971 on pLAI3, corresponding to positions 4757 to 4935 on pNL4-3) is capable of increasing transduction efficiency. The present invention includes the discovery that while this small fragment is not sufficient to increase the transduction efficiency, a larger 545 base pair fragment (positions 4551 to 5096 in pNL4-3), or yet larger fragments containing it, were capable of increasing transduction as part of the present invention.

Additional examples of viral vector constructs that may be used in the present invention are found in U.S. Pat. No. 5,885,806, which is hereby incorporated by reference as if fully set forth. The constructs in U.S. Pat. No. 5,885,806 are merely examples that do not limit the scope of vectors that efficiently transduce cells. Instead, the constructs provide additional guidance to the skilled artisan that a viral vector for use with the present invention may contain minimal sequences from the wild-type virus or contain sequences up to almost the entire genome of wild-type virus, yet exclude an essential nucleic acid sequence required for replication and/or production of disease. Methods for determining precisely the sequences required for efficient transduction of cells are routine and well known in the art. For example, a systematic incorporation of viral sequences back into a “basic” vector or deleting sequences from vectors that contain virtually the entire HIV genome, such as cr2HIVs, is routine and well known in the art.

Furthermore, placing sequences from other viral backbones into viral vectors of interest, such as the cytomegalovirus (CMV), is also well known in the art. Regardless of the actual viral vector used, various accessory proteins encoded by, and sequences present in, the viral genetic material may be left in the vector or helper genomes if these proteins or sequences increase transduction efficiency in certain cell types. Numerous routine screens are available to determine whether certain genetic material increases transduction efficiency by incorporating the sequence in either the vector or helper genomes. One embodiment of the invention is to not include accessory proteins in either the vector or helper genomes. This embodiment does not exclude embodiments of the invention where accessory proteins and other sequences are left in either the vector or a helper genome to increase transduction efficiency.

The viral vectors used in the present invention may also result from “pseudotype” formation, where co-infection of a cell by different viruses produces progeny virions containing the genome of one virus encapsulated within an outer layer containing one or more envelope protein of another virus. This phenomenon has been used to package viral vectors of interest in a “pseudotyped” virion by co-transfecting or co-infecting a packaging cell with both the viral vector of interest and genetic material encoding at least one envelope protein of another virus or a cell surface molecule. See U.S. Pat. No. 5,512,421. Such mixed viruses can be neutralized by anti-sera against the one or more heterologous envelope proteins used. One virus commonly used in pseudotype formation is the vesicular stomatitis virus (VSV), which is a rhabdovirus. The use of pseudotyping broadens the host cell range of the virus by including elements of the viral entry mechanism of the heterologous virus used.

Pseudotyping of viral vectors and VSV for use in the present invention results in viral particles containing the viral vector nucleic acid encapsulated in a nucleocapsid which is surrounded by a membrane containing the VSV G protein. The nucleocapsid can contain proteins normally associated with the viral vector. The surrounding VSV G protein containing membrane forms part of the viral particle upon its egress from the cell used to package the viral vector. Examples of packaging cells are described in U.S. Pat. No. 5,739,018. In another embodiment of the invention, the viral particle is derived from HIV and pseudotyped with VSV G protein. Pseudotyped viral particles containing the VSV G protein can infect a diverse array of cell types with higher efficiency than amphotropic viral vectors. The range of host cells include both mammalian and non-mammalian species, such as humans, rodents, fish, amphibians and insects.

The viral vector for use in the transduction methods of the invention can also comprise and express one or more nucleic acid sequences under the control of a promoter present in the virus or under the control of a heterologous promoter introduced into the vector. The promoters may further contain insulatory elements, such as erythroid DNAse hypersensitive sites, so as to flank the operon for tightly controlled gene expression. Example of promoters include the HIV-LTR, CMV promoter, PGK, U1, EBER transcriptional units from Epstein Barr Virus, tRNA, U6 and U7. While Pol II promoters are useful, Pol III promoters may also be used. The use of tissue specific promoters are also one embodiment. For example, the beta globin Locus Control Region enhancer and the alpha & beta globin promoters can provide tissue specific expression in erythrocytes and erythroid cells. Another embodiment is to use cis-acting sequences that are associated with the promoters. For example, the U1 gene may be used to enhance antisense gene expression where non-promoter sequences are used to target the antisense or ribozymes molecule to a target spliced RNA as set out in U.S. Pat. No. 5,814,500, which is hereby incorporated by reference.

Of course any cis acting nucleotide sequences from a virus may be incorporated into the viral vectors of the invention. For example, cis acting sequences found in retroviral genomes can be used. For example, cis-acting nucleotide sequence derived from the gag, pol, env, vif, vpr, vpu, tat or rev genes may be incorporated into the viral vectors of the invention to further increase transduction efficiency. A cis acting sequence does not need to encode an expressed polypeptide; does not need to be expressed as a polypeptide or part thereof due to genetic alteration, such as deletion of a translational start site; can encode only a portion or fragment of a larger polypeptide; or can be a mutant sequence containing one or more substitutions, additions, or deletions from the native sequence. An example of a cis acting sequence is the cPPT (central polypurine tract) sequence identified within the HIV pol gene.

The one or more nucleic acid sequences in the viral vectors of the invention may be found in the virus from which the vector is derived or be a heterologous sequence. The can be a full-length or partial sequence that is or encodes a gene product of interest. Such sequences and gene products can be biologically active agents capable of producing a biological effect in a cell. Examples of such agents include proteins, ribonucleic acids, enzymes, transporters or other biologically active molecules.

In one embodiment, the agent is a protein, such as a toxin, transcription factor, growth factor or cytokine, structural protein, or a cell surface molecule. The protein may contain one or more domains for which no function has been identified and may be homologous to the transduced cell. Additionally, the protein may be absent, deficient or altered in the cell to be transduced. Alternatively, the protein may be a transdominant negative mutant or a decoy to prevent a natural protein from carrying out its normal activity in the transduced cell.

For example, the nucleic acid sequence may code for a ribozyme that binds, cleaves and destroys RNA expressed, or to be expressed, in the transduced cell. Alternatively, the nucleic acid sequence may code for an antisense molecule designed to target a particular nucleic acid sequence and result in its degradation. The vector contained sequence may be overexpressed, inducibly expressed, or under cellular or viral regulatory transcription control in the transduced cell. Depending on the intended use, the heterologous sequence may encode any desired protein including a marker for transduced cells. Such markers include selectable markers such as a particular resistance phenotype, such as neomycin, MDR-1 (P-glycoprotein), O⁶-methylguanine-DNA-methyltransferase (MGMT), dihydrofolate reductase (DHFR), aldehyde dehydrogenase (ALDH), glutathione-S-transferase (GST), superoxide dismutase (SOD) and cytosine deaminase. See Koc et al., which is hereby incorporated by reference, for a review.

In the methods of the invention, the cells to be transduced are exposed to contact with the at least one molecule that binds the cell surface before, after, or simultaneously with application of the viral vector. For example, the cells can be cultured in media with CD3 and CD28 antibodies (coated onto the surface of the culture dish or immobilized on beads present in the culture) before, after, or in the presence of the viral vector to be transduced. The cells can be exposed to immobilized CD3 and/or CD28 only after or only upon initial contact with the viral vector. Under these conditions, the cells are not exposed to cell surface binding molecule(s) prior to actual transduction with the viral vector. In embodiments where contact with a cell surface binding molecule occurs after exposure of the cells to a viral vector (transduction), the contact can occurs within three days of transduction, or alternatively within one to two days after transduction.

Incubation or contacting of the cells with the viral vector may be for different lengths of time, depending on the conditions and materials used. Factors that influence the incubation time include the cell, vector and MOI (multiplicity of infection) used, the molecule(s) and amounts used to bind the cell surface, whether and how said molecule(s) are immobilized or solubilized, and the level of transduction efficiency desired. For example, the incubation is for about eight to about 72 hours, or about 12 to about 48 hours. In another embodiment, the incubation or contacting is for about 24 hours and is optionally repeated once. In another embodiment, the incubation or contacting is for about 24 hours to about 36 hours.

Contact between the cells to be transduced and a viral vector occurs at least once, but it may occur more than once, depending upon the cell type. For example, high efficiency transduction of CD34 positive stem cells have been accomplished with multiple transductions with vector. Another method of the invention is to simultaneously introduce a viral vector in combination with a cell surface binding molecule (e.g., CD3 and/or CD28 antibodies or a FLT-3 ligand, TPO or Kit ligand) and avoid changing the medium for between about one and about eight days after transduction. Alternatively, the medium is not changed for three days post transduction. Transduction can proceed for as long as the conditions permit without the process being significantly detrimental to the cells or the organism containing them. Additional examples of cell surface binding proteins for such use include those described hereinabove.

The MOI used can be from about 1 to about 400, or less than 500. The MOI can be from about 2 to about 50. Alternatively, the MOI is from about 10 to about 30, although ranges of from about 1 to about 10, about 20, about 30, or about 40 are also contemplated. Alternatively the MOI of about 20 or about 0.5 to 10. Furthermore, the copy number of viral vector per cell should be at least one. However, many copies of the vector per cell may also be used with the above described methods. An exemplary number of copies per cell is from about 1 to about 100. The desired copy number is the minimum copy number that provides a therapeutic, prophylactic or biological impact resulting from vector transduction or the most efficient transduction.

For therapeutic or prophylactic applications, a desired copy number is the maximum copy number that is tolerated by the cell without being significantly detrimental to the cell or the organism containing it. Both the minimum and maximum copy number per cell will vary depending upon the cell to be transduced as well as other cells that may be present. The optimum copy number is readily determined by those skilled in the art using routine methods. For example, cells are transduced at increasing increments of concentration or multiplicities of infection. The cells are then analyzed for copy number, therapeutic or biological impact and for detrimental effects on the transduced cells or a host containing them (e.g., safety and toxicity).

After incubation with the viral vector in vitro, the cells may be cultured in the presence of the cell surface binding molecule(s) for various times before the cells are analyzed for the efficiency of transduction or otherwise used. Alternatively, the cells may be cultured under any conditions that result in cell growth and proliferation, such as incubation with interleukin-2 (IL-2) or incubation with the cell surface binding molecule(s) followed by IL-2. Post transduction incubation may be for any period of time, for example, from about one to about seven to ten days. Longer periods of time, such as about 14 days, may also be used, although periods that are detrimental to cell growth are not desired. In embodiments of the invention where the cells are cultured with the cell surface binding molecule(s) before incubation with the viral vector, the culture times may range from about 24 to about 72 hours, or about 24 hours.

Such pre-transduction culturing may be compared to stimulation of cells, with cytokines and/or mitogens for example, prior to transduction as taught in the art. The present invention includes advantages resulting from the avoidance of such stimulation. For example, stimulation expands the numbers of cells through proliferation to result in many more cells post-stimulation than pre-stimulation. Transduction of this expanded set of cells requires much more viral vector and related transduction materials (e.g., containers, media, cytokines etc), increasing the associated cost. Furthermore, the stimulation of cells affects their quality for further applications. Movassagh et al. describe the use of a three day pre-transduction stimulation that resulted in deterioration of the T cell repertoire diversity after transduction and further culturing. Additionally, pre-transduction stimulation removes the advantage available from the transduction of cells that are not actively dividing.

The efficiency of transduction observed with the present invention is from about 50-100%. For example, the efficiency is at least about, 50-75% or about 75 to 90%. Other embodiments of the invention are where transduction efficiency is at least about 90 to 100%. Additional embodiments have transduction efficiencies of at least 91, 92, 93, 94, 95, 96, 97, 98, 99 and 100%.

In addition to the above, the transduced cells may be used in research or for treatment or prevention of disease conditions in living subjects. An example of a research use is the structure-function studies described by Unutmaz et al. Therapeutic uses for the transduced cells include the introduction of the cells into a living organism. For example, unstimulated primary T cells isolated from an individual infected with, or at risk of being infected with HIV, may be first transduced by a vector, like that described in U.S. Pat. No. 5,885,806, using the present methods and followed by injection of the transduced cells back into the individual. Alternatively, the cells may be used directly for the expression of a heterologous sequence present in the viral vector.

When used as a part of HIV therapy or prophylaxis, the vector may encode a toxin or other anti-viral agent that has been adapted for anti-HIV applications. Alternatively, the vector may encode an agent designed to target HIV, such as transdominant negative mutants of the tat, rev, nef, vpu, or vpr genes. In other applications the transduced cell may be corrected to express an appropriate globin gene to correct sickle cell anemia or thalassaemia. Immune cells may also be transduced to modulate their immune function, their response to antigen, or their interactions with other cells. The skilled artisan is aware of the above uses for the present transduction methods as well as numerous other uses and applications known in the art.

The invention provides processes for manufacturing autologous T cells and transducing T cells. Using the methods of the invention, high transduction levels are achieved when cells are cultured in solid plastic flasks versus plastic bags. In one aspect, for large scale transductions, 10-layer cell factories are used. In one aspect, 2-fold reduction of vector volume is necessary for efficient transduction of T cells on clinical scale. In one aspect, the vector (for the transduction of the T cell) is added twice with a 24 hour interval to further increase transduction.

In one aspect, in growing T cells lower concentrations of oxygen and slightly lower pH than in the regular media. The invention found that T cells expand better in the presence of lower concentrations of oxygen and slightly lower pH than in the regular media. In one aspect, N₂/O₂ 90%/10% is used to culture T cells, in contrast to regular air (˜20% of O₂). In one aspect, CO₂ concentration in air mixture is raised to 10%, up from usual 5%, to reduce pH. This changes in gas mixture allowed for higher expansion rates.

In one aspect, about 100 billion cells in the end of the expansion, perfusion of media is used. We found that in order to have about 100 billion cells in the end of the expansion, perfusion of media had to be used. In one aspect, a 50 L perfusion bag is used, as previously used 20 L bags were not able to support enough cells. In one aspect, perfusion is started as soon as cell concentration goes over 0.5×10⁶ cells/ml with the approximate speed of 3 L/day. In one aspect, the speed is increased by approximately 2-fold next day with simultaneous increase of rocking speed and angle by 1 unit.

In one aspect, during the harvest T cells are cooled down to increase their viability after subsequent freezing and thawing. In some applications this needs to be done because the harvest of large amount of cells takes long time and cells survive better at lower temperature. In one aspect, cells are transferred in smaller 10 L bags and placed in the refrigerator for cooling. Refrigerated buffer should be used for washing cells.

The present invention is directed to methods, and compositions related thereto, for the stable transduction of cells with viral vectors to efficiencies of greater than about 75%. Stably transduced cells may be distinguished from transiently transduced, or pseudotransduced cells, after about seven to ten days, or optionally after about 14 days, post transduction. The methods relate to the fact that contact of the cells to be transduced with at least one molecule that binds the cell surface increases the efficiency of stable transduction. Surprisingly, the contacting step may occur after the transduction step. Even more surprisingly, the highest levels of stable transduction were seen when transduction occurred first followed by contact with immobilized cell surface binding molecules.

The methods of the invention comprise the step of transduction with a viral vector in combination with contact with a cell surface binding molecule. As noted above, the contact may occur before, after or at the same time as transduction with the vector. The invention is broadly applicable to any cell, and the use of any cell surface binding molecule. Cells for use with the present methods include unstimulated primary cells, which are freshly isolated from an in vivo source as well as cell lines, which may have been previously cultured for various times in the presence of factors which maintain them in a proliferating state. When cell lines are used, they may be first cultured in the absence of stimulatory factors prior to transduction with the present methods.

In the case of primary cells, they are first obtained from an in vivo source followed optionally by selection for particular cell types. For example, if primary CD4+ and/or CD8+ T cells are to be used, peripheral blood (PB) or cord blood (“CB” from an umbilical source) samples are first obtained followed by enrichment for CD4+ and/or CD8+ cell types. Standard magnetic beads positive selection, plastic adherence negative selection, and/or other art recognized standard techniques may be used to isolate CD4+ and/or CD8+ cells away from contaminating PB cells. Purity of the isolated cell types may be determined by immunophenotyping and flow cytometry using standard techniques.

After isolation, the primary cells may be used in the present methods to be transduced with viral vectors at efficiencies of greater than 50%, or greater than 75%. The invention is most advantageously used with primary lymphocytes, such as T cells, transduced with an HIV-1 based vector capable of expressing heterologous genetic material of interest. Another use is with primary hematopoietic stem cells, such as CD34 positive cells. In cases where the heterologous genetic material is or encodes a therapeutic or prophylactic product for use in vivo to treat or prevent a disease, the transduced primary cell can be introduced back into an in vivo environment, such as a subject. As such, the invention contemplates the use of the transfected cells in gene therapy to treat, or prevent, a disease by combating a genetic defect or targeting a viral infection.

The invention is also contemplated for use in efficiently transducing cells for determining the function of a gene, expressing genes efficiently in mammalian cells, expressing genetic libraries (cDNA libraries and genetic antisense or ribozymes libraries) for functional screening for genes of interest, use in protein-protein or protein-nucleic acid two-hybrid like detection strategies, gene trapping approaches, high-throughput gene screening analysis with a microarray or protein array, or studies employing SAGE, proteomics and other functional analytical methods.

For the transduction of primary cells in a mixed population, the above isolation/purification steps would not be used. Instead, the cell to be transduced would be targeted by selection of at least one appropriate cell surface molecule or moiety found on that cell type and the preparation of one or more molecules capable of binding said moiety. The cell surface moiety may be a receptor, marker, or other recognizable epitope on the surface of the targeted cells. Once selected, molecules that interact with the moiety, such as specific antibodies, may be prepared for use in the present invention.

For example, CD4+ and/or CD8+ cells can either be first purified and then transduced by the methods of the invention with the use of immobilized CD3 and CD28 antibodies or alternatively be transduced as part of a mixed population, like peripheral blood cells (PBCs) or peripheral blood mononuclear cells (PBMNCs), by use of the same antibodies. Hematopoietic stem cells in total white blood cell populations, which may be difficult to purify or isolate, may be transduced in the mixed populations by use of immobilized CD34 antibodies.

The cell surface binding molecules of the invention may target and bind any moiety found on the surface of the cell to be transduced. The moieties are found as part of receptors, markers, or other proteinaceous or non-proteinaceous factors on the cell surface. The moieties include epitopes recognized by the cell surface binding molecule. These epitopes include those comprising a polypeptide sequence, a carbohydrate, a lipid, a nucleic acid, an ion and combinations thereof.

Examples of cell surface binding molecules include an antibody or an antigen binding fragment thereof and a ligand or binding domain for a cell surface receptor. The cell surface binding molecule may itself be a polypeptide, a nucleic acid, a carbohydrate, a lipid, or an ion. The molecule can be an antibody or a fragment thereof, such as a F_{ab} or Fᵥ fragment. Alternatively, the molecule is not used in a soluble form but is rather immobilized on a solid medium, such a bead, with which the cells to be transduced may be cultured, or the surface of a tissue culture dish, bag or plate, upon which the cells to be transduced may be cultured. In one embodiment for the transduction of CD4+ or CD8+ cells, monoclonal antibodies that recognize CD3 and/or CD28 may be used in a cell culture bag in the presence of a viral vector.

The present invention includes compositions comprising a cell surface binding molecule for use as part of the disclosed methods. An exemplary composition comprises the molecule and a viral vector to be transduced, optionally in the presence of the cells to be transduced. The viral vectors may be derived from any source, for example, retroviral vectors. For example, they can be lentiviral vectors. An exemplary lentiviral vector is one derived from a Human Immunodeficiency Virus (HIV), for example, HIV-1, HIV-2, or chimeric combinations thereof. Of course different viral vectors may be simultaneously transduced into the same cell by use of the present methods. For example, one vector can be a replication deficient or conditionally replicating retroviral vector while a second vector can be a packaging construct that permits the first vector to be replicated/packaged and propagated. When various viral accessory proteins are to be encoded by a viral vector, they may be present in any one of the vectors being transduced into the cell. Alternatively, the viral accessory proteins may be present in the transduction process via their presence in the viral particles used for transduction. Such viral particles may have an effective amount of the accessory proteins co-packaged to result in an increase in transduction efficiency. In one embodiment, the viral vector does not encode one or more of the accessory proteins.

A viral vector for use in the transduction methods of the invention can also comprise and express one or more nucleic acid sequences under the control of a promoter. In one embodiment of the invention, a nucleic acid sequence encodes a gene product that, upon expression, would alleviate or correct a genetic deficiency in the cell to be transduced. In another embodiment, the nucleic acid sequence encodes or constitutes a genetic antiviral agent that can prevent or treat viral infection. By “genetic antiviral agent”, it is meant any substance that is encoded or constituted by genetic material. Examples of such agents are provided in U.S. Pat. No. 5,885,806. They include agents that function by inhibiting viral proteins, such as reverse transcriptase or proteases; competing with viral factors for binding or target sites; or targeting viral targets directly for degradation, such as in the case of ribozymes and antisense constructs. Other examples of genetic antiviral agents include antisense, RNA decoys, transdominant mutants, interferons, toxins, nucleic acids that modulate or modify RNA splicing, immunogens, and ribozymes, such as “hammerhead” and external guide sequence (EGS) mediated forms thereof.

Alternatively, a viral vector can encode a marker for transduced cells. In the examples presented in the figures and below, green fluorescent protein (GFP) is the marker encoded by the viral vector transduced into CD4+ cells. Other markers include those listed above. Detection of GFP may serve to identify the number of functionally transduced cells, which were not only transduced with the vector, but were also able to functionally express GFP to levels that could be detected by FACS analysis. It should be noted that the detection may not represent the actual number of transduced cells since some cells may have been transduced with the vector but express GFP at levels that are below the limits used in FACS detection.

An alternative approach to detecting transfection efficiency is with the polymerase chain reaction (PCR). For example, TaqMan PCR can be used to determine the actual number of copies of stably integrated viral vector in a transduced cell.

The cells to be transduced may be exposed to contact with the viral vector either before, after or simultaneously with contact with the cell surface binding molecule. Thus the cells can be first exposed to the vector for a period of time followed by introduction of the cell surface binding molecule. Such cells may be newly isolated or prepared primary cells that have not been intentionally stimulated to enter the cell cycle. Alternatively, the cells can be first exposed to the cell surface binding molecule for a period of time followed by contact with the viral vector. After contact with the vector, excess vector does not need to be removed and the cells can be cultured under conditions conducive to cell growth and/or proliferation. Such conditions may be in the presence of the cell surface binding molecule or other stimulatory/activating factors, such as cytokines and lymphokines in the case of T cells. Alternatively, excess vector may be removed after contact with the cell and before further culturing.

Another embodiment of the invention is to culture the cells in the presence of both viral vector and cell surface binding molecule simultaneously. Such cells are need not be previously stimulated. After a period of time, the cells are cultured under growth or proliferation inducing conditions such as the continued presence of the cell surface binding molecule or other stimulatory/activating factors. Alternatively, excess vector may be removed before further culturing.

In any of the above combinations of viral vector and cell surface binding molecule administration, incubation with the vector can be optionally repeated at least once. Contact with the vector can also be repeated more than once, such as twice, thrice, four times, or more.

Incubation of the cells to be transduced with the viral vector may be for different lengths of time, depending on the conditions and materials used. Factors that influence the incubation time include the cell, vector and MOI (multiplicity of infection) used, the molecule(s) and amounts used to bind the cell surface, whether and how said molecule(s) are immobilized, and the level of transduction efficiency desired. In one embodiment of the invention, the cells are T lymphocytes, the vector HIV based, the MOI is about 20, the cell surface binding molecules are CD3 and CD28 antibodies immobilized on beads, and the resultant efficiency at least 93%. As would be evident to the skilled person in the art, some of the above factors are directly correlated while others are inversely correlated. For example, a decrease in the MOI will likely decrease the level of efficiency while efficiency can likely be maintained if an increased amount of cell surface binding molecules is used.

The length of incubation viral vector and the cells to be transformed can be, for example, for 24 hours and optionally repeated once for lymphocytes and up to four times for hematopoietic stem cells. Similarly, and in embodiments where the cells are incubated with the cell surface binding molecule before introduction of the viral vector, the incubation may be for about 12 hours to about 96 hours. Incubation with a cell surface binding molecule can occur simultaneously with contact of the cells with the viral vector. Under such circumstances, the cell surface binding molecules may be left in contact with the cells when the vector is introduced. Alternatively, excess cell surface binding molecules may be first removed from the culture before introduction of the vector to the cells.

After contact with the vector, the cells are cultured under conditions conducive to their growth or proliferation. For example, the conditions are continued culturing in the presence of the cell surface binding molecules. Alternatively, the cells are initially cultured with the cell surface binding molecule followed by substitution with media containing another factor conducive to cell growth, such as interleukin-2. Yet another embodiment would be to remove both the excess cell surface binding molecule and the excess vector followed by culturing in the presence of a factor conducive to growth or proliferation as well as enhancing further vector transduction. Such factors include mitogens such as phytohemaglutinin (PHA) and cytokines, growth factors, activators, cell surface receptors, cell surface molecules, soluble factors, or combinations thereof, as well as active fragments of such molecules, alone or in combination with another protein or factor, or combinations thereof.

Examples of additional factors include epidermal growth factor (EGF), transforming growth factor alpha (TGF-alpha), angiotensin, transforming growth factor beta (TGF-beta), GDF, bone morphogenic protein (BMP), fibroblast growth factor (FGF acidic and basic), vascular endothelial growth factor (VEGF), PIGF, human growth hormone (HGH), bovine growth hormone (BGH), heregulins, amphiregulin, Ach receptor inducing activity (ARIA), RANTES (regulated on activation, normal T expressed and secreted), angiogenins, hepatocyte growth factor, tumor necrosis factor beta (TNF-beta), tumor necrosis factor alpha (TNF-alpha), angiopoietins 1 or 2, insulin, insulin growth factors I or II (IGF-I or IGF-2), ephrins, leptins, interleukins 1, 2, 3, 4, 5, 6, 7, 8, 9, 10, 11, 12, 13, 14, or 15 (IL-1, IL-2, IL-3, IL-4, IL-5, IL-6, IL-7, IL-8, IL-9, IL-10, IL-11, IL-12, IL-13, IL-14, or IL-15), G-CSF (granulocyte colony stimulating factor), GM-CSF (granulocyte-macrophage colony stimulating factor), M-CSF (macrophage colony stimulating factor), LIF (leukemia inhibitory factor), angiostatin, oncostatin, erythropoietin (EPO), interferon alpha (including subtypes), interferons beta, gamma, and omega, chemokines, macrophage inflammatory protein-1 alpha or beta (MIP-1 alpha or beta), monocyte chemotactic protein-1 or -2 (MCP-1 or 2), GRO beta, MIF (macrophage migration inhibitory factor), MGSA (melanoma growth stimulatory activity), alpha inhibin HGF, PD-ECGF, bFGF, lymphotoxin, Mullerian inhibiting substance, FAS ligand, osteogenic protein, pleiotrophin/midkine, ciliary neurotrophic factor, androgen induced growth factor, autocrine motility factor, hedgehog protein, estrogen, progesterone, androgen, glucocorticoid receptor, RAR/RXR, thyroid receptor, TRAP/CD40, EDF (erythroid differentiating factor), Fic (growth factor inducible chemokine), IL-IRA, SDF, NGR or RGD ligand, NGF, thymosine-alpha1, OSM, chemokine receptors, stem cell factor (SCF), or combinations thereof. As evident to one skilled in the art, the choice of culture conditions will depend on knowledge in the art concerning the cells transduced as well as the subsequent intended use of the cells. For example, the combination of IL-3, IL-6 and stem cell factor would not be a choice for transduced cells that are to be used in human transplantation. Similarly, the choice of culture conditions would desirably not be to the detriment of cell viability or transduction efficiency.

The post transduction incubation is for a period of, for example, about four hours, or for about one to about seven to ten days. The post transduction incubation can also be for a period of, for example, from about 16 to about 20 hours or for about four, about five or about six days. About fourteen days of post-transduction incubation is also contemplated.

The efficiency of transduction observed with the present invention is from about 50-100%. The efficiency can be at least about 50-75%, or at least about 75 to 90%. Other embodiments of the invention are where transduction efficiency is at least about 90 to 95%. Additional embodiments have transduction efficiencies of at least 91, 92, 93, 94, 95, 96, 97, 98, 99 and 100%.

In addition to the above, the transduced cells may be used in research or for treatment of disease conditions in living subjects. As part of the invention are therapeutic uses for the transduced cells to produce gene products of interest or for direct introduction into a living organism as part of gene therapy. For example, and as exemplified below, primary T cells can be isolated and transduced with a viral vector. Successful transduction is indicated by the production or overproduction of a gene product encoded by the vector or generation of a phenotype conferred by the vector. As such, primary T cells can be first transduced with a vector containing, and capable of expressing, desirable or useful nucleic acid sequences, and then returned to an in vivo environment such as a living subject. For example, the living subject is an individual infected with, or at risk of being infected with HIV-1.

In another embodiment, the T cells are transduced with genes or nucleic acids capable of conditionally killing the T cell upon introduction into a host organism. This has applications in allogenic bone marrow transplantation to prevent graft versus host disease by killing T cells with a pro-drug approach.

Alternatively, the primary cells can be deficient in a gene product, and the deficiency correctable by the transduced viral vector. Such cells would be reintroduced into the living subject after transduction with the vector.

Thus, both in vitro and ex vivo applications of the invention are contemplated. For transfers into a living subject, the transduced cells are, for example, in a biologically acceptable solution or pharmaceutically acceptable formulation. Such a transfer may be made intravenously, intraperitoneally or by other injection and non-injection methods known in the art. The dosages to be administered will vary depending on a variety of factors, but may be readily determined by the skilled practitioner. There are numerous applications of the present invention, with known or well designed payloads in the viral vector, where the benefits conferred by the transduced genetic material will outweigh any risk of negative effects.

Initially, the total number of transduced cells transferred would be from about 10⁴ to about 10¹⁰. As such, 10⁵, 10⁶, 10⁷, 10⁸, or 10⁹ cells may be used. The actual numbers will vary depending on the cells being transduced. Multiple transfers, if required, of transduced cells are another embodiment. Furthermore, conditioning of the host prior to the transfer of transduced cells, if required, is another embodiment. Conditioning regimens are known in the art; an example is the regimen(s) for bone marrow transplantation.

The amount of cells that can be grown in a multilayer vessel or flask is at least about 100 million cells, or about at least 70 million cells, or about at least 80 million cells, or about at least 90 million cells.

The present invention provides highly efficient methods and compositions related thereto for the stable transduction of cells with viral vectors and viral particles. The invention provides novel manufacturing facilities and manufacturing processes for cell processing of transduced cells, e.g., lentiviral vector-modified cells, such as autologous CD4+ T cells, e.g., the exemplary VRX496-transduced CD4+ T cells described herein.

In one aspect, the processes of the invention for manufacturing autologous T cells comprise isolation of lymphocytes, e.g., by frozen apheresis of blood, washing, e.g., in a CytoMate™ as described below, then CD4+ enrichment followed by CD8 depletion, followed by transduction with virus, e.g., a lentivirus. Further processing is described below and illustrated in the figures herein.

In one aspect, the starting material for the production of Autologous VRX496-transduced CD4+ T cells is peripheral blood mononuclear cells (PBMC). PBMC are obtained from an HIV-infected subject during leukapheresis. The leukapheresis procedure can occur in a blood collection facility using an automated cell separator.

In one aspect, the cells are washed to remove plasma and magnetically labeled (incubated) with CD4 microbeads (Miltenyi Biotech, Germany), which have been developed for the separation of human cells based on the expression of the CD4 antigen. During Phase I clinical study, the starting material underwent ficoll density gradient separation by low speed centrifugation to remove plasma and then underwent COBE (Baxter) washing and resuspension in working buffer. The washed cell material was then incubated with CD8 high density microparticles (CD8-HDM nickel beads) (Biotransport) for subsequent magnetic separation using an Eligix Magnetic Cell Separation System.

For Phase II clinical study, cell washing to remove plasma can be performed using the CYTOMATE™ Cell Processing System (Miltinyi Biotech, Germany). The CYTOMATE™ Cell Processing System is a stand-alone, closed and automated device for washing and concentrating cellular products, and fluid transfer applications. It enables efficient cell washing with low cell loss and high viability. The system features a disposable tube set that creates a closed system fluid path for cell processing in a cGMP environment. It also makes fluid transfer flexible, fast and accurate. Solutions can be transferred to and from single or multiple containers, all within a closed system fluid path.

In one aspect, immune globulin solution (Immune Globubin Intravenous, USP, Grifols) is added to prevent non-specific cell binding during incubation of the added CD4+ microbeads (Miltinyi Biotech). In one aspect, the end product bag (CD4 microbead incubated cell suspension) is heat sealed and the bag is removed and placed under the biological safety hood.

In one aspect, an Eligix™ Cell Separation System is used for CD8 depletion. For Phase II clinical study, CD4+ positive selection can be performed via a CliniMACS™ magnetic cell separation system. This system uses a sterile CliniMACS™ disposable set consisting of (1) a transfer pack container, (2) plasma transfer sets with female luer adapters for connection to a buffer bag and a cell suspension bag and (3) plasma transfer sets with female luer adapters for connection to a positive selection bag and a waste collection bag.

In one aspect, spiking of the sterile disposal sets' buffer and cell suspension lines to the respective bags can be done under a biological safety hood to maintain sterility. Once the phosphate buffered saline (PBS) buffer and cell suspension lines have been spiked, the disposable set can be attached to the CliniMACS™ and the CD4 magnetic labeled cell suspension can be run through the CliniMACS™. The collected positive fraction can be used to continue with the process.

In one aspect, PBS to X-VIVO-15 media (Cambrex; Walkersville, MD) exchange is accomplished via the CytoMate™. In one aspect, the end product bag is removed, heat sealed and placed under the biological safety hood. A transfer set with female luer adapter can be attached to the product bag and a 5 cc sample is obtained by syringe for QC testing for: the percentage of CD3+ CD8+ cells and CD3+ CD4+ cells, cell viability, cell number, and pre-expansion HIV Gag measurement. In one aspect, cell production stops until QC results are obtained. If the cells meet specification, production continues with cell transduction.

In one aspect, CD3/CD28 co-stimulation beads (Dynal beads, Oslo, Norway, coated with anti-CD3 (OKT3) and anti-CD28 (UPenn monoclonal antibody 9.3) are added to the CD4+ T Cell suspension followed by the addition of the VRX496 viral vector product. In one aspect, the whole mixture of CD4+ T cells, X-VIVO+5% Human Serum Albumin, IL2, NAC, CD3/CD28 microbeads and VRX496 vector suspension (5% WN) are added to a Nunc™ cell factory coated with RetroNectin (Takara Bio, Japan) and the cell factory put into a humidified, 37° C., 5% CO₂ incubator. VRX496 vector suspension (5% WN) is once again added the next day. In one aspect, the cells are incubated with vector for 3 days, then transferred to WAVE™ cell bag and placed into a Wave™ Bioreactor (WAVE™ Biotech LLC, Bridgewater, N.J.).

The WAVE bioreactor has a special rocking platform. The rocking motion of this platform induces waves in the culture fluid. These waves provide mixing and oxygen transfer, resulting in a perfect environment for cell growth that can easily support over 20×10⁶ cells/ml. Tubing leads on the bags and a variety of connecting devices (connection will be via spike connectors and welds produced via the Terumo Sterile Connecting Device) allow the cells to be grown in a closed system with minimal risk of contamination.

To remove vector, on the 4^{th} day, the cells can be washed two times with X-VIVO 15 using the CytoMate™ cell washer.

The cultures are maintained for 7 to 12 days until it is time to harvest them. The cells are counted at least every other day and fresh medium is added to maintain the cells at an approximate density of 0.5-1.5×10⁶ cells/ml. Antiretroviral drugs (Norvir, Abbot Laboratories, and Retrovir, GlaxoSmithKline) (1 μmol/L) are added to inhibit HIV replication while the cells are in culture. Other types of antiretroviral drugs can be used. One of skill in the art would know how to choose and administer the appropriate antiretroviral. At about day 10, the cells are ready for harvesting. A post-expansion HIV Gag measurement is performed to insure that the post-expansion HIV copies are not greater than pre-expansion HIV copies. From the pre-harvest cells, a sample is taken to test for mycoplasma.

In one aspect, the CD3/CD28 microbeads are removed by passing the culture bag over a MaxSep™ magnet (Baxter). The beads are retained on the magnets and the cells are poured into another bag. The cells are assayed for residual beads.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The following drawings are illustrative of aspects of the invention and are not meant to limit the scope of the invention as encompassed by the claims.

The patent or application file contains at least one drawing executed in color. Copies of this patent or patent application publication with color drawing(s) will be provided by the Office upon request and payment of the necessary fee.

Figure are enclosed. Like reference symbols in the various drawings indicate like elements.

FIG. 1 shows exemplary vessels or flasks that can be used in the methods and systems of the invention.

FIGS. 2A and 2B show maps of pN2cGFP and pN1GFP(cPT), respectively. Various restriction enzyme sites are indicated as well as the components derived from HIV. The pN2cGFP construct contains the GFP coding sequence operably linked to a CMV (cytomegalovirus) promoter, thus controlling GFP expression. The pN1GFP(cPT) construct is also referred to as pN1(cpt)CGFP below and contains the cPPT from the HIV pol gene. These constructs are used in the examples described below.

FIG. 3 shows the results of transduction of primary T cells using beads coated with immobilized CD3 and CD28 antibodies. The cells were either contacted with vector before contact with the beads (Panel A), contacted with the beads prior to contact with the vector (Panel B), or simultaneously contacted with both vector and beads (Panel C). The flow cytometry results based on fluorescence from GFP encoded by the transduced vector indicate that the cells in Panels A-C were transduced 90.70, 87.19, and 79.14%, respectively.

FIG. 4 shows a comparison of transduction using either IL-2 and PHA-P or bead immobilized CD3 and CD28 antibodies to stimulate CD4+ cells before contact with viral vector. The use of immobilized antibodies resulted in transduction efficiencies of over 95% each time. The use of IL-2 and PHA resulted on efficiencies of only 70.2 to 84.5%.

FIG. 5 is a depiction of the frequency of human CD4+ T cell transduction using the present methods. Fifteen days post transduction, a comparison of flow cytometry analysis of control cells versus cells transduced with a vector capable of expressing green fluorescent protein (GFP) at a MOI of 20 shows that about 93% of the transduced cells also exhibit green fluorescence.

FIG. 5 shows the results of FACS analysis for CD4+ and GFP+ cells at 14 days post transduction using either IL-2 and PHA-P or bead immobilized CD3 and CD28 antibodies. Approximately 93% of the antibody treated cells remained stably transduced after 14 days. Only about 75% of cells treated with IL-2 and PHA remained stably transduced after that time.

FIG. 6 shows the results of cells transduced with different viral vectors.

FIG. 7 shows the effect from the use of different MOIs on transfection efficiency.

FIG. 8 demonstrates the stable transduction of CD34+ cells prepared from umbilical cord blood after multiple transductions with a viral vector in the presence of cell surface binding molecules. Over 88% of cells remained positive after over 6 weeks post transduction.

FIG. 9, panels A-D, shows the efficiency of long term transduction after transplantation to SCID (severe combined immunodeficiency) mice. After approximately eight weeks, an average of over 91% of the transduced cells, which continued to mature, remained positive for expression of the transduced GFP marker.

FIG. 10, panels A and B, shows the efficiency of dendritic cell transduction after seven days.

FIG. 11 shows a schematic representation of the purification process and associated equipment for isolation or enrichment of CD4 T lymphocytes from subject apheresis product, by using either CD8 depletion (CD4 enrichment) or CD4 positive selection (CD4 isolation).

FIG. 12 shows a schematic representation of the cell processing manufacturing including transduction, expansion and cryopreservation, with associated equipment. Product used in this figure comes from the initial isolation procedure described above in FIG. 11.

FIG. 13 shows a schematic overview of the “no expansion” cell processing procedure and associated equipment. CD4 positive selection is shown here, but CD4 positive selection, or CD4 enrichment via depletion of non-CD4 cells can also be used. “Smart vector” refers to a specially packaged lentiviral vector as described in U.S. provisional No. 60/585,464, that due to proteins incorporated in its envelope has an enhanced ability to bind, stimulate, and transduce cells.

FIG. 14 shows a schematic overview of the “no expansion” transduction kit for cell processing, and associated equipment. This procedure allows for a closed system of isolation and transduction of cells for decentralized distribution. This process does not contain an expansion step. The enrichment of CD4 cells is performed using the Rosette-Sep, which is a method for depletion of non-CD4 cells.

FIG. 15 shows a schematic overview of the “in line” transduction process. This process uses the closed system of the apheresis machinery to perform any purification (this may or not be used), and transduction for direct reinfusion to the subject.

FIG. 16A-C shows a flow chart describing apheresis, transduction and expansion of primary cells which are then re-infused into a subject. In addition, cell processing steps and associated quality control measures are shown.

FIG. 17 shows an exemplary lentiviral vector. Specifically, a HIV based vector derived from WT HIV.

FIG. 18 shows several retroviral vectors: VRX496, VRX494, and VRX577. (Top) This figure is a schematic representation of pN1cptASenv, (VRX496), depicting elements and the regions of WT HIV from which it was derived. The numbers in the vector refer to the size of the genetic elements. The vector expresses a 937 bp antisense segment targeted against the HIV envelop gene (ASenv). The antisense payload is Tat and Rev dependent and thus expressed only after HIV infects vector-containing cells. The HIV derived elements include the 5′ and 3′ long terminal repeat (LTR), a packaging signal (v), tRNA primer binding site (PBS), central polypurine tract and central termination sequence (cPPT & CTS), splice acceptor and donor sites (SA/SD), Tat-dependent HIV promoter (P), Gag gene, rev response element (RRE), and 3′ polypurine tract (PPT). Engineered elements include a stop codon in gag. Gtag is a non-coding marker sequence from GFP.

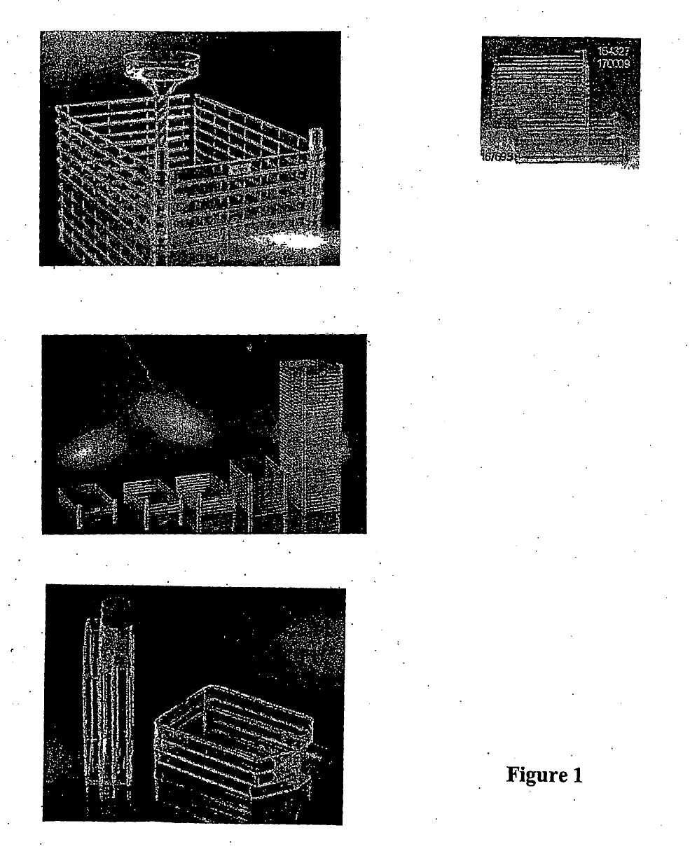

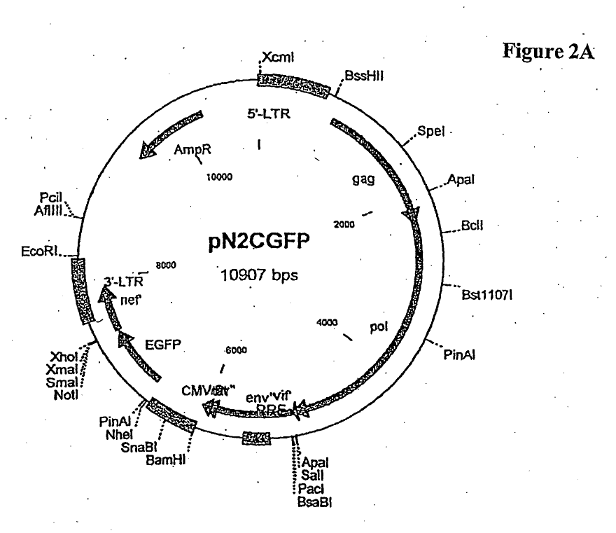

(Bottom) This is a schematic representation of pVRX577 (VIRPAC), the helper packaging construct. VRX496 is Pseudotyped with a vesicular stomatitis virus protein G (VSV-G) envelope. Gag and Pol are expressed under the control of the cytomegalovirus (CMV) promoter, Rev under the control of the rev response element derived from HIV-2 (RRE-2), which is used to reduce homology between VRX496 and VIRPAC, Tat under the control of an internal ribosomal entry site (IRES), and VSV-G expressed by an elongation factor 1α (EF-1α)/human T cell lymphotrophic virus (HTLV) chimeric promoter. VSV-G is separated from the other packaging genes for safety by several pause signals and a cis-acting ribozyme derived from the tobacco mosaic ringspot virus (sTobRV+Rz) that will cleave any readthrough RNA. Sequences of rev and tat genes were partially degenerated to reduce homology with the vector.

FIG. 19 shows how VRX496 antisense DNA integrates into the DNA of the subject's T-cell. FIG. 19 is a description of lentiviral vector VRX496 and its mechanism of action utilizing its antisense payload.

FIG. 20 shows how VRX496 destroys HIV RNA production. FIG. 20 is a description of lentiviral vector VRX496 and its mechanism of action utilizing its antisense payload.

FIG. 21 shows how use of VRX496 solves the HIV resistance problem. This graphic represents a comparison between the number of mutations needed for resistance to develop for anti HIV drugs, versus the number of mutations needed for resistance to develop against VRX496. HIV either gets destroyed by the antisense payload or it mutates to levels where the virus is not fit to cause disease.

FIG. 22 shows the superior efficiency of gene transfer using a viral vector tagged with a green fluorescent protein. FIG. 22 shows the level of gene transduction into primary T cells by flow cytometry.

FIG. 23 shows how VRX496 inhibits WT HIV replication from in primary lymphocytes collected from HIV(+) patients.

FIG. 24 shows the survival advantage of VRX496 transduced cells compared to unprotected cells 21 days post HIV infection.

FIG. 25 shows an overview of an examplary clinical process using autologous cell therapy for the treatment of HIV. FIG. 25 is a schematic representation of VIRxSYS′ Phase I clinical trial.

FIG. 26 shows baseline characteristics of study subjects.

FIG. 27 shows CD4 cell counts for Phase I clinical trials of VRX496 infused subjects.

FIG. 28 shows HIV viral loads for Phase I clinical trials of VRX496 infused subjects.

FIG. 29 shows the use of several retroviral drugs: AZT, ddC/S aqinavir, D4T/3Te/Crixivan, Norvir/Amprenivir/ddI/Adefovir, Sustiva/Ziagen/Kaletra/3T e/Viread. FIG. 29 shows the viral load history for subject No. 2 of VIRxSYS Phase I clinical trial.

FIG. 30 shows sustained engraftment and persistence of VRX496 modified CD4 T cells. FIG. 30 is a line graph showing VRX496 vector persistence assessed beginning 20 minutes after infusion of VRX496 modified CD4+ cells, and then at 72 hours, 1, 2, 3, and 6 weeks, and 3, 6, 9, and 12 months. PBMC's were collected at the indicated time points, and DNA analysis was performed for VRX496 detection using real time PCR. The limit of quantification is 100 vector copies per 10⁶ PBMC's. At the 1-year time point, patient No. 4 has a frequency of engraftment of 0.04% (400 copies) after being undetectable at the 9-month time point.

FIG. 31 shows Immune Function Analysis: IFN-g ELISPOT Env. FIG. 31 is a bar graph showing HIV-1 env specific effector cells IFN-γ secretion. Blood samples were obtained at baseline and 3 and 6 months post gene transfer therapy. PBMC's were isolated from subjects and HIV-1 positive controls subjects (n=25) by a standard ficoll separation technique. IFN-γ production following HIV-1 env in vitro stimulation of PBMC's was assessed for an env antigen specific response by a standard ELISPOT. The mean ±95% confidence interval for the control subjects plotted.

FIG. 32 shows Immune Function Analysis: IFN-g ELISPOT—Gag. FIG. 32 is a bar graph showing HIV-1 gag specific effector cells IFN-γ secretion. Blood samples were taken before and 3 and 6 months post gene transfer therapy. PBMC's were isolated from subjects and HIV-1 positive controls subjects (n=25) by a standard ficoll separation technique. IFN-γ production following HIV-1 gag in vitro stimulation of PBMC's was assessed for an gag antigen specific response by a standard ELISPOT. The mean ±95% confidence interval for the control subjects plotted.

FIG. 33 shows adjustments for increased vector production and yield made during Phase I and Phase II production.

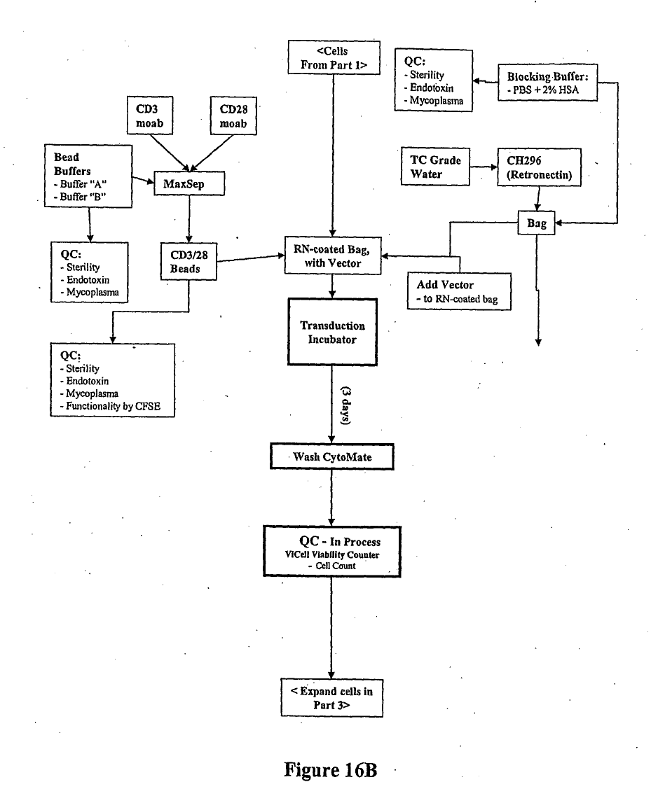

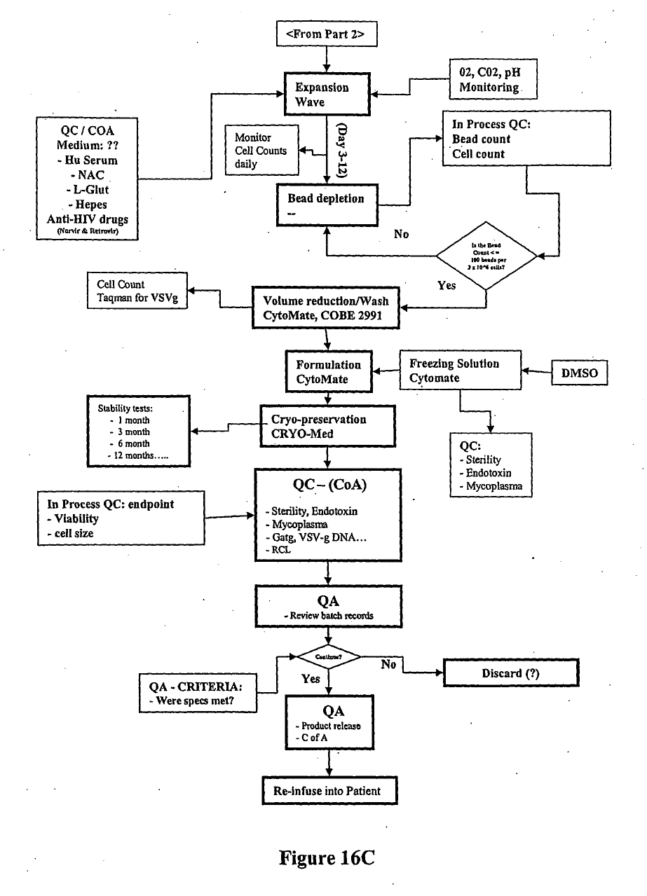

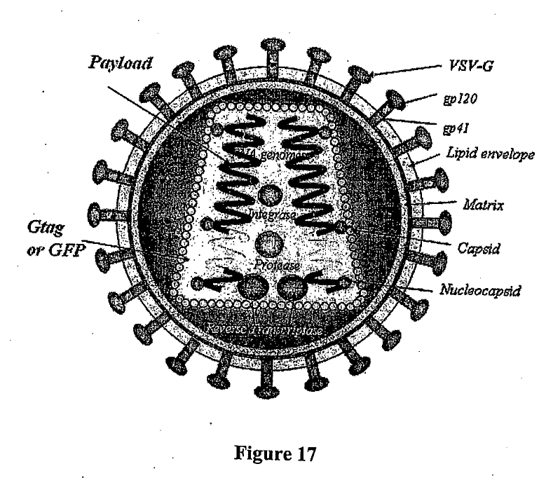

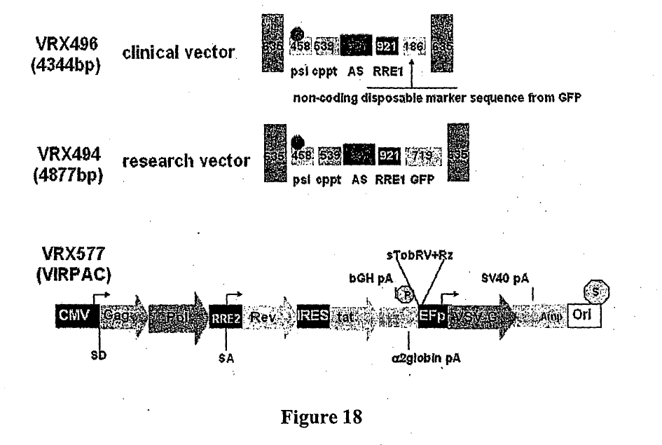

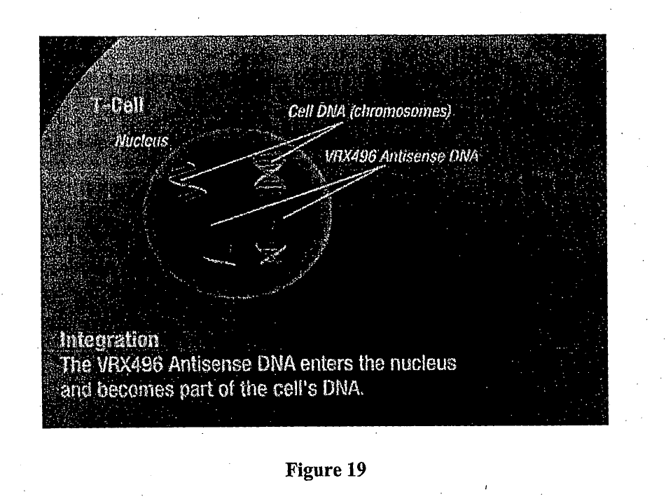

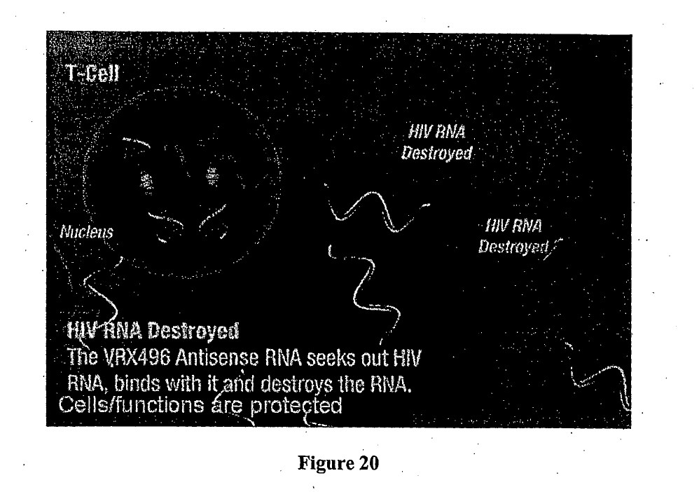

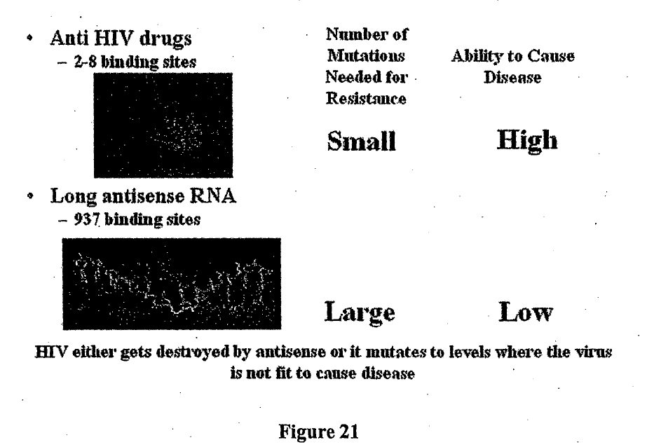

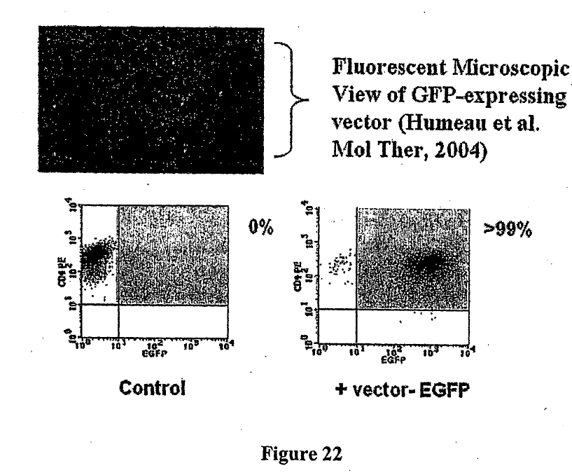

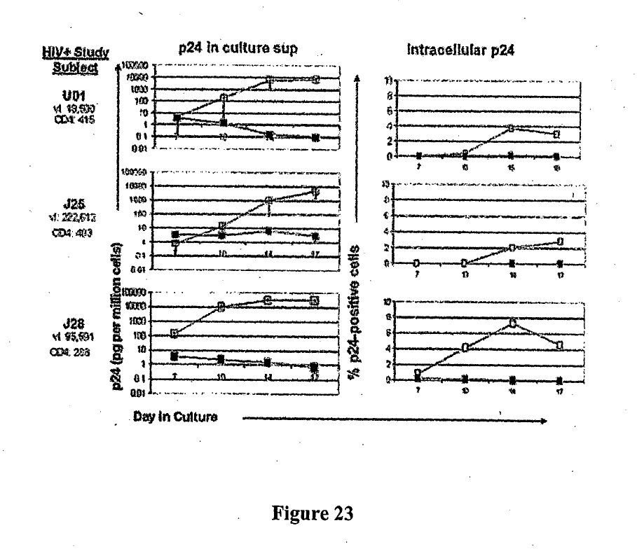

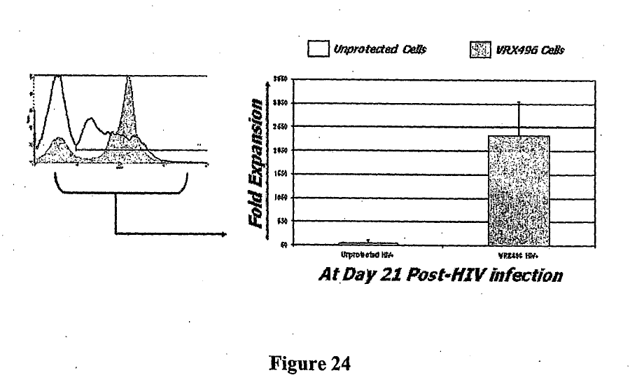

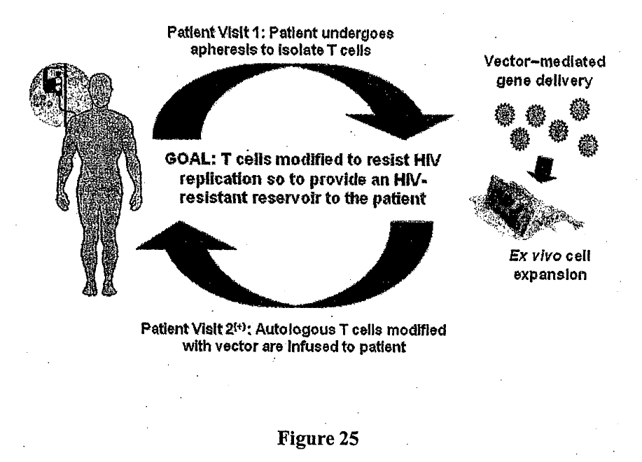

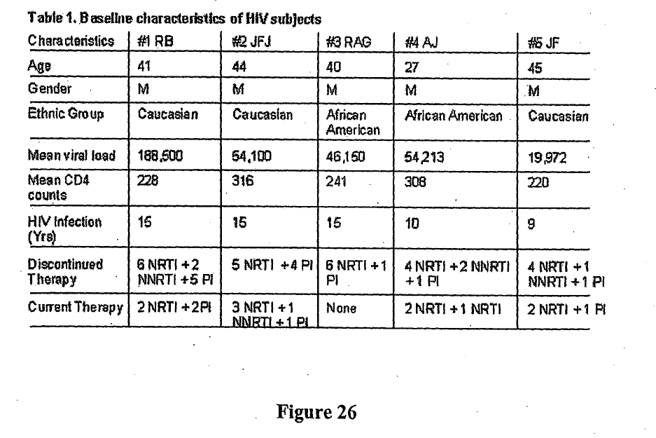

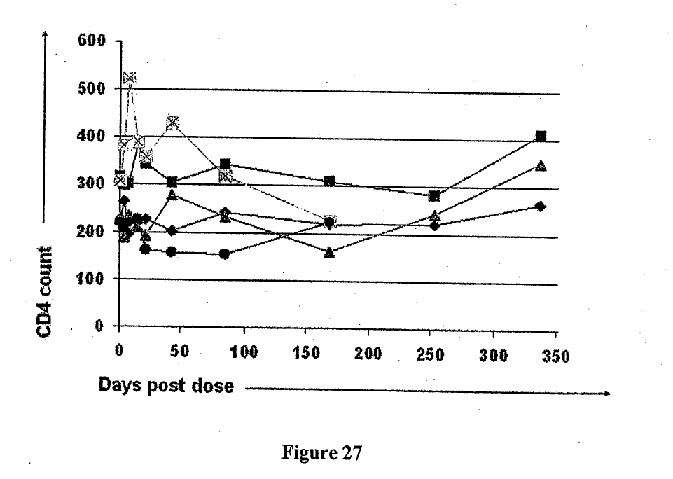

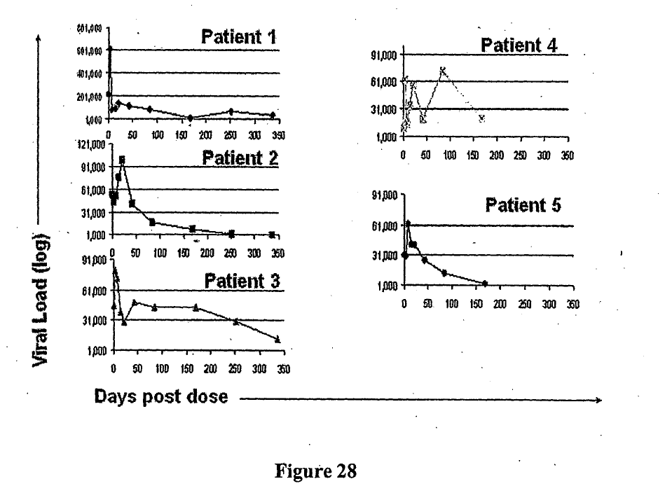

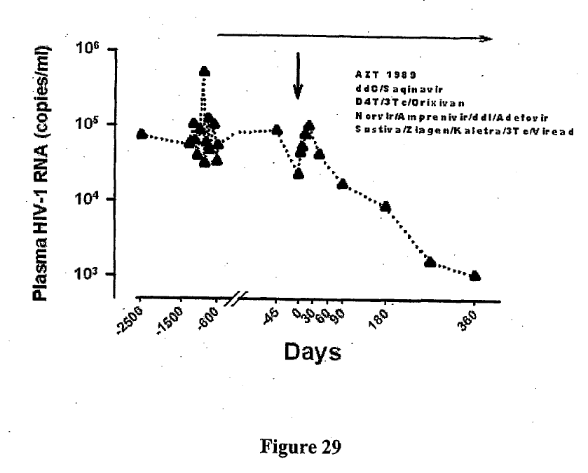

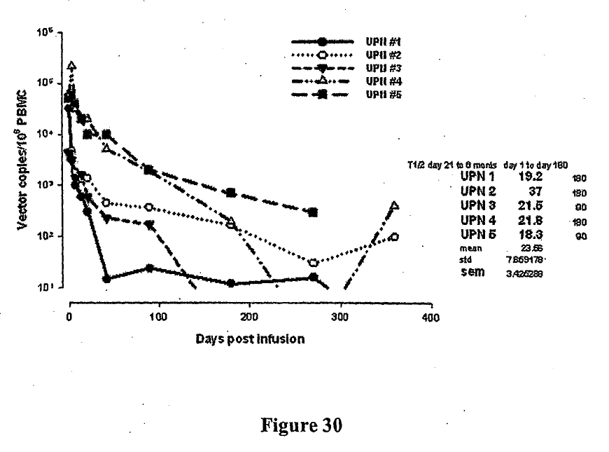

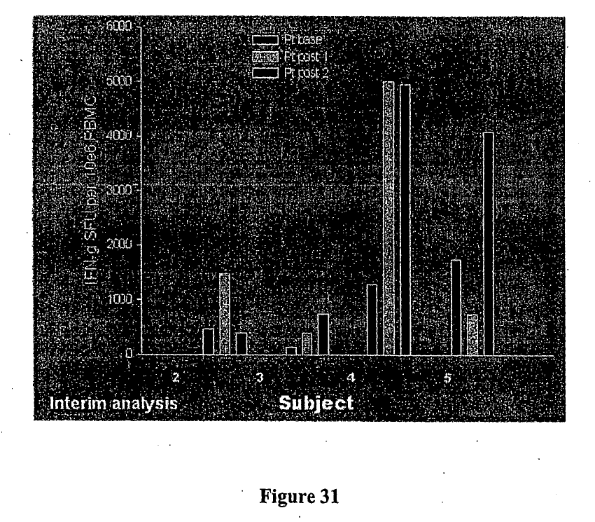

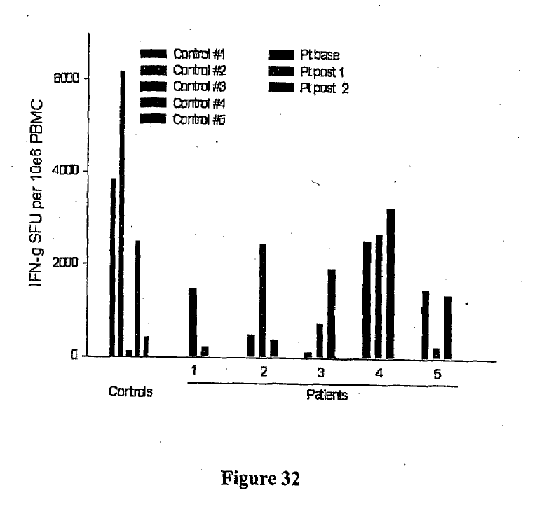

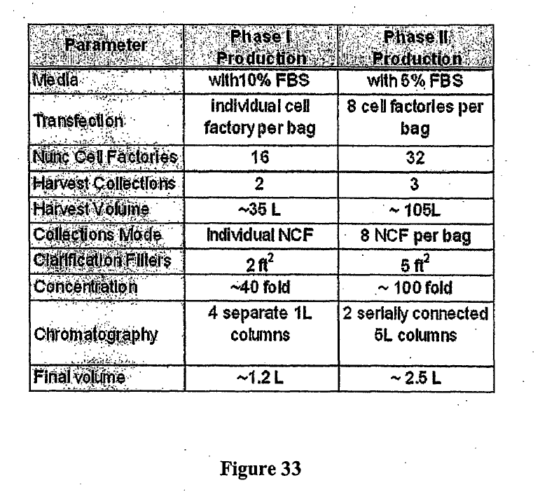

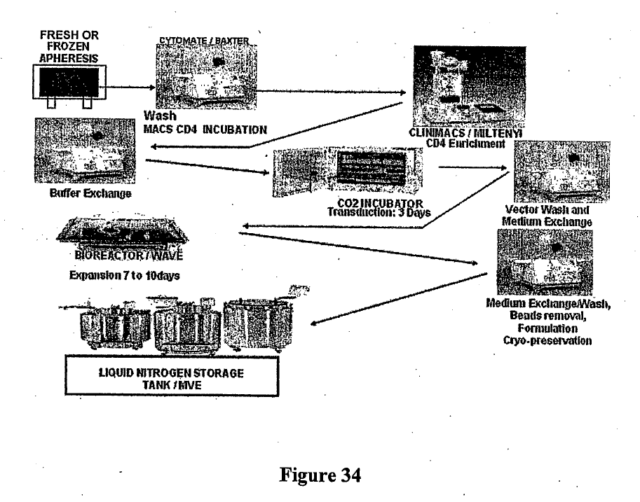

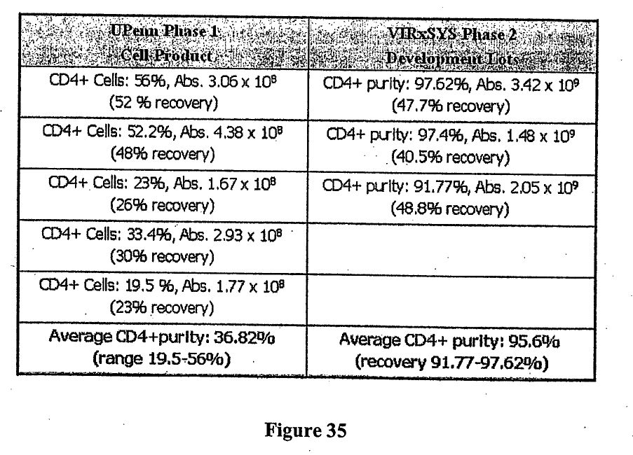

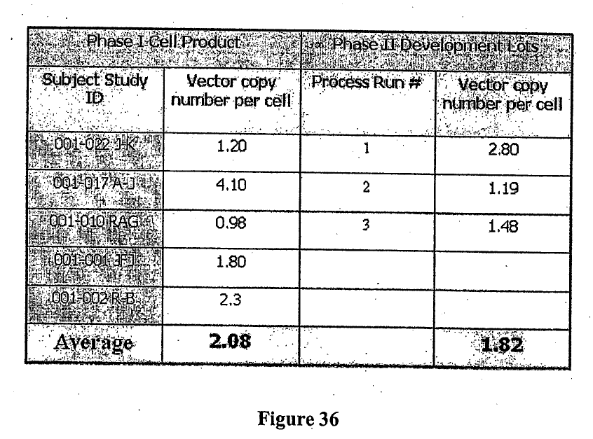

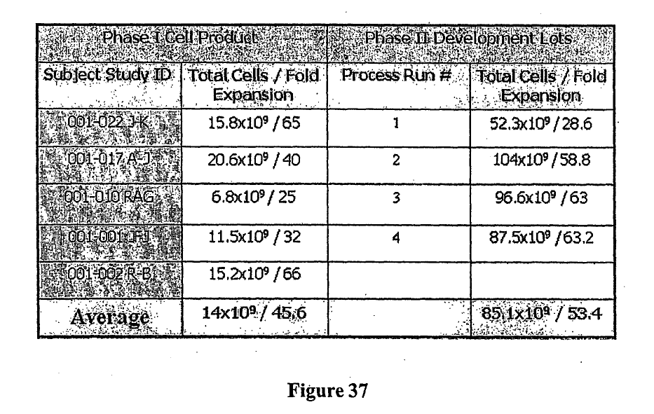

FIG. 34 shows an exemplary autologous T cell manufacturing process. FIG. 34 is a schematic representation of clinical grade, large scale cell processing related to FIGS. 16 A, B and C. Fresh or frozen apheresis products can be used in the process described in FIG. 39.

FIG. 35 shows a comparison of Phase I CD4+ product purity with VIRxSYS Phase II development process.

FIG. 36 shows a comparison of Phase I VRX496 copy number with VIRxSYS Phase II VRX496 viral copy number.

FIG. 37 shows a comparison of Phase I cell product expansion with VIRxSYS Phase II cell product expansion.

Having now generally described the invention, the same will be more readily understood through reference to the following examples which are provided by way of illustration, and are not intended to be limiting of the present invention, unless specified.

### EXAMPLE 1

### Preparation of Primary CD4+ T Cells

CD4+ T cells were isolated from peripheral blood using standard protocols with slight modification. More specifically, contaminating monocytes were depleted by attachment. Non adherent cells were placed in the presence of magnetic beads coated with anti-CD4 antibodies for positive selection of CD4+ cells. The magnetic beads were removed and CD4+ cells isolated.

The highly purified CD4+ cells were confirmed to be greater than 90% by flow cytometry.

### EXAMPLE 2

### Transduction of Primary CD4+ T Cells with Variations in Time of Contact with a Cell Surface Binding Molecule

Transduction Before Cell Surface Binding

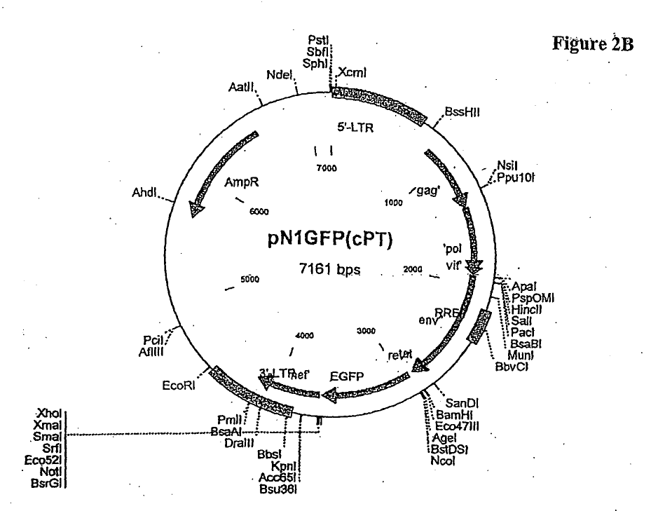

Primary CD4+ cells (about 500,000) were cultured with pN2cGFP at a MOI of 20 for 24 hours followed by addition of αCD3 and αCD28 coated beads to the culture for an additional seven days. FIG. 2 contains a map of pN2cGFP.

Transduction after Cell Surface Binding

Primary CD4+ cells (about 500,000) were cultured for 24 hours with αCD3 and αCD28 coated beads for 24 hours followed by introduction of pN2cGFP at a MOI of 20 to the culture for an additional 24 hours. The cells were washed to remove excess vector followed by incubation in vector free media containing the beads for an additional seven days.

Simultaneous Transduction and Cell Surface Binding

Primary CD4+ cells (about 500,000) were cultured with pN2cGFP at a MOI of 20 for 24 hours in the presence of αCD3 and αCD28 coated beads. The cells were washed to remove excess vector followed by incubation in vector free media containing the beads for an additional seven days.

Optional Protocol Substitutions

Other viral vectors may be substituted for pN2cGFP. Additionally, the transduction may be repeated for a total of two times prior to removal of excess vector. Moreover, the αCD3 and αCD28 coated beads may be substituted by interleukin-2 (10 ng/ml) and PHA-P (3 mg/ml) after transduction and removal of excess vector. After seven days, the media is replace with PHA-P free media containing interleukin-2 (10 ng/ml) and the incubation continued for an additional seven days.

Alternatively, after seven days of post-transduction incubation with αCD3 and αCD28 coated beads, the cells are washed and incubation continued in the presence of interleukin-2 (10 ng/ml).

### EXAMPLE 3

### Post-Transduction Analysis

Post-transduction and seven or 14 days after incubation, the cells were analyzed by flow cytometry for CD4+ and/or green fluorescent protein (GFP).

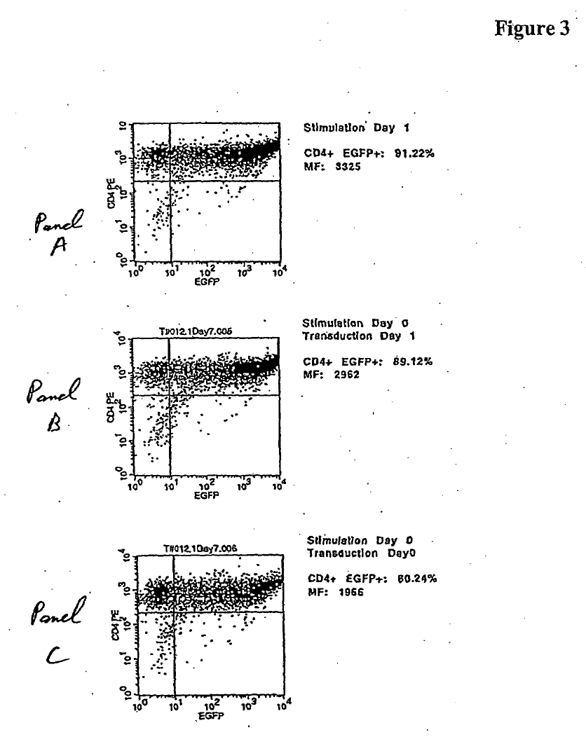

A comparison of the above three transduction protocols is shown in FIG. 3. Contact with bead immobilized CD3 and CD28 antibodies after transduction with pN2cGFP at an MOI of 20 resulted in about 91% efficiency. Contact with beads before transduction resulted in about 89% efficiency, and simultaneous bead contact and transduction resulted in about 80% efficiency. In this experiment, the CD4+ T cells were selected by adherence monocyte depletion, CD14 MACS depletion and CD4 MACS enrichment. The antibodies were immobilized as described below. Contact with the vector was at 37° C. and 5% CO₂. The culture conditions were at 500,000 CD4+ T cells per ml in Yssel's medium supplemented with 2% human serum albumin. FACS analysis was on day seven post selection. MF refers to mean fluorescence.

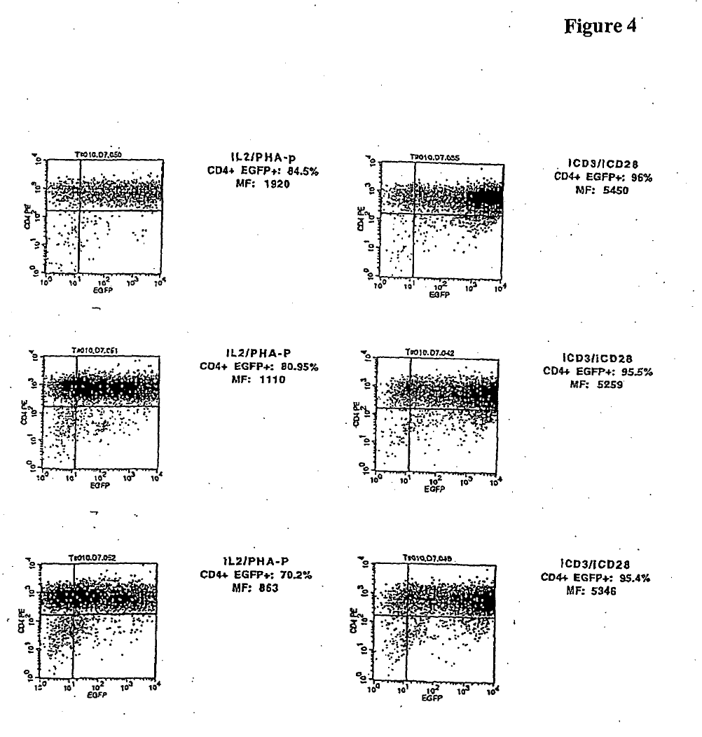

The results for an experiment after seven days comparing different stimulation conditions are shown in FIG. 4. CD4+ cells were treated with either IL-2 and PHA-P or bead immobilized CD3 and CD28 antibodies for 24 hours followed by one round of transduction with pN2cGFP at an MOI of 20. In side by side comparisons, the use of immobilized antibodies resulted in transduction efficiencies of over 95% each time (indicated by cells positive for both CD4 and GFP). By comparison, the results with IL-2 and PHA stimulation resulted on efficiencies of only 70.2 to 84.5%. FACS analysis was on day seven post selection.

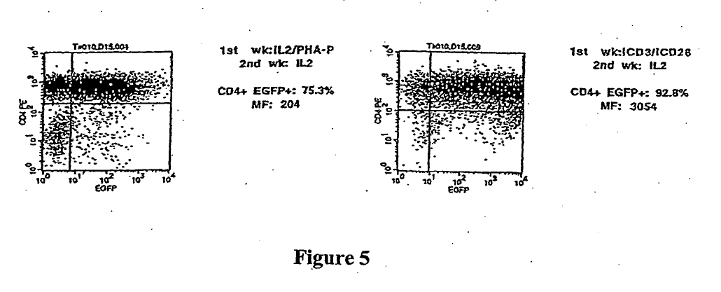

FIG. 5 shows the results of a similar experiment at 15 days post selection. Cells were again treated with either IL-2 and PHA-P or bead immobilized CD3 and CD28 antibodies for 24 hours followed by one round of transduction with pN2cGFP at an MOI of 20. The PHA-P and beads were removed on day 7 after transduction, and the cells were cultured with only IL-2 at 500,000 cells per ml. until day 15 post selection. Approximately 93% of the cells were positive for both CD4 and GFP after the use of immobilized antibodies. Only about 75% of cells treated with IL-2 and PHA remained positive for both CD4 and GFP. These results indicate that a small fraction of the cells detected as positive after seven days (FIG. 4) may have been due to “pseudotransfection.”

### EXAMPLE 4

### Different Vectors Stably Transduce Cells at High Efficiencies

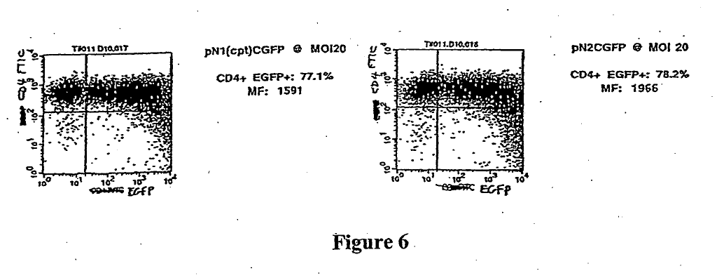

This example is a comparison of vectors used for transduction. pN2cGFP contains the entire gag and pol coding sequence while pN1(cpt)cGFP contains the 4551-5096 partial (non-coding) pol sequence. As can be seen from the results, shown in FIG. 6, both vectors show very efficient transduction of primary CD4 cells after simultaneous stimulation with bead immobilized CD3 and CD28 antibodies and vector at an MOI of 20. FACS analysis was performed on day 10 post selection.

### EXAMPLE 5

### Effect of MOI on Transfection Efficiency

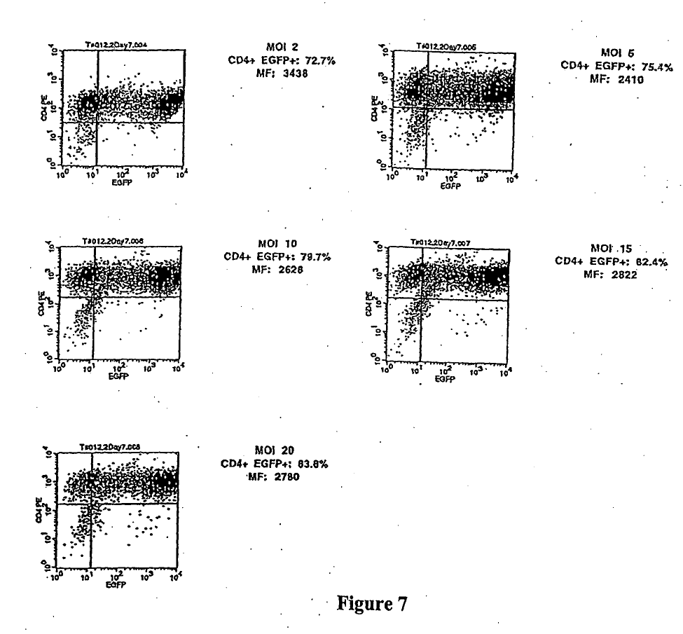

The effect of different MOIs are shown in FIG. 7, where the use of MOIs from 2 to 20 resulted in transduction efficiencies from 72.7 to 83.8%. Cells were contacted with bead immobilized CD3 and CD28 antibodies for 24 hours before transduction with pN1(cpt)CGFP at different MOIs.

### EXAMPLE 6

### Transduction of CD34 Positive Cells

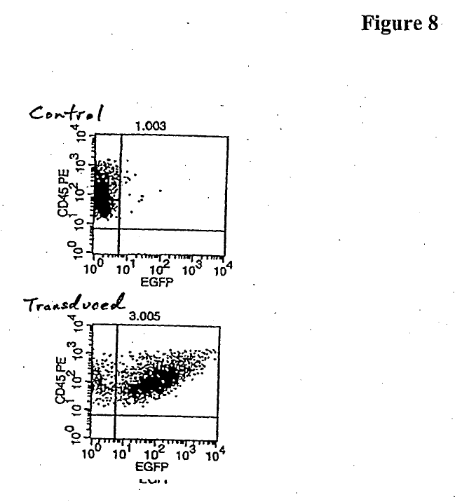

CD34 positive cells were prepared from cord blood and transduced four times with pN1cptGFP simultaneously in the presence of FLT-3 ligand, TPO and Kit ligand (10 ng/ml each). The cells were cultured for five weeks in long term culture (LTC-IC) and then the cells cultured in methylcellulose for 10 days prior to analysis (the results are from an elapsed time of over 6 weeks in culture). The results in FIG. 8 analyze mature CD45 positive cells that result from the CD34 immature cells. Control cells show no significant transduction while the vector transduced cells show over 88% of cells as CD45 and GFP positive.

### EXAMPLE 7

### Long Term Transduction of CD34 Positive Cells

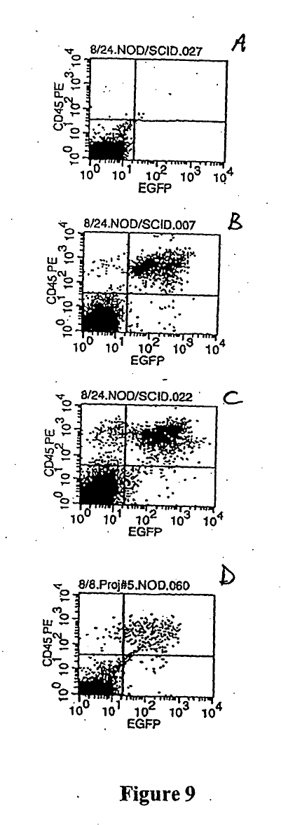

CD34 positive cells were transduced with pN1(cpt)GFP as described above and transplanted to the bone marrow of partially irradiated SCID mice. After eight weeks, cells were isolated and analyzed for CD45 bearing mature human cells and GFP expression by FACS. The results are shown in FIG. 9, panels A to D.

Panel A shows the results with a control mouse transplanted with human cells not transduced with vector.

Panel B shows the results with a mouse transplanted with cells transduced with cells transduced with pN1(cpt)GFP vector at a MOI of 50 for 4 sequential days in the presence of 100 ng/ml of FLT-3 ligand, TPO and Kit ligand. This mouse shows a striking 96.3% transduction efficiency of transduced human cells (CD45 positive cells) 8 weeks after transduction. The level of human cell engraftment in this mouse was 11.1%, consistent with previously reported results.

Panels C and D show the results with two other mice treated as in panel B. The results confirm reproducibility of high efficiency transduction with 87.8% and 89.6% of CD45 positive cells also being GFP positive.

The average efficiency is 91.2%, which reflects long term stable transduction.

### EXAMPLE 8

### Immobilization of Cell Surface Binding Molecules

This example describes the direct linkage of CD3 (B-B11) antibodies and CD28 (B-T3) antibodies to epoxy dynal beads for use in the examples below.

1. Prepare 0.1 borate solution by dissolving 0.618 g. boric acid in 95 ml of tissue culture grade water. Mix well and adjust pH to 9.5 using highest quality NaOH. Bring final volume to 100 ml and sterilize via a 0.2 μm filter. Seal container and store at 4° C.

2. Add antibody to above borate solution at a concentration of 150 μg/ml. For both B-B11 and B-T3 antibodies, add 75 μg of each per ml of borate solution. Bring volume to 1 ml total. Borate concentration should not be below 0.05 M after adding antibody. For each 1 ml of borate/antibody solution, add 4×10⁸ Epoxy beads.

3. Incubate 24 hours at 37° C. on rotating wheel.

4. Wash beads three times for 10 min. each at 22° C. with bead wash media: phosphate buffered saline without calcium and magnesium, 3% human serum albumin, 5 mM EDTA, and 0.1 sodium azide.

5. Wash beads once for 30 min at 22° C.

6. Wash overnight at 4° C.

7. Replace with fresh bead wash media, resuspend beads to 2×10⁸ beads/ml. IgG coated beads are stable for at least 6 months at 4° C.

### EXAMPLE 9

### Transduction of Dendritic Cells

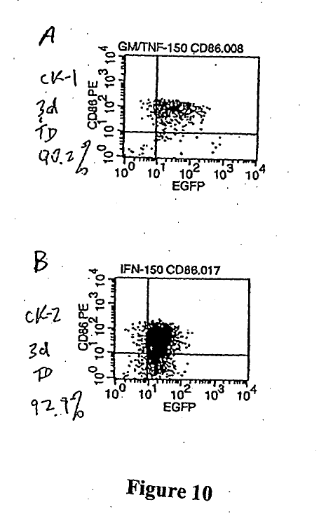

Monocytes from peripheral blood were isolated and then transduced for three consecutive days at an MOI of 50 with pN2cGFP using two simultaneous cytokine conditions: GM-CSF (800 units/ml), IL-4 (500 units/ml) and TNF-alpha (100 units/ml) or GM-CSF (500 units/ml) and interferon-alpha (800 units/ml). FIG. 10, panel A, shows the results seven days post transduction where the first cytokine condition resulted in 90.2% efficiency. Cells transduced with vector under the second cytokine conditions show a 92.9% efficiency after seven days (panel B). CD86 is only one possible marker for dendritic cells, and it should be noted that CD86 negative cells can also be dendritic cells.

### EXAMPLE 10

### Manufacturing Facility

This summary provides information describing a new manufacturing facility and manufacturing process for cell processing of lentiviral vector-modified cells, and specifically for Autologous VRX496-transduced CD4+ T Cells.

During a Phase I clinical study, Autologous VRX496-transduced CD4+ T Cells were manufactured at the University of Pennsylvania (UPenn) Cell and Vaccine Production Facility (CVPF), (Levine, et al., 2002).

For a Phase II clinical study, the autologous VRX496-transduced CD4+ T cell product will be manufactured. This process allows for the unprecedented expansion of over 100-fold of HIV-infected CD4 T cells modified by a lentiviral vector.

Submitted in this summary are:

A description of the manufacturing facility, including the general facility layout,

A description of the new manufacturing process, including the starting materials, in-process and release quality control testing, and stability, and

Data demonstrating consistency in manufacture and comparability data to the UPenn CVPF process.

### EXAMPLE 11

### General Facility Description

The GMP manufacturing area and the quality control (QC) testing areas are contained and separated from the other establishment areas and from each other by physical barriers. The manufacturing area and QC testing areas are also restricted and controlled via card-key lock.

The intended uses of the two clean rooms are for clinical cGMP production of VRX496 lentiviral vector and the corresponding cGMP ex vivo transduction of subject's cells with this vector. The vector production clean room is a multiple product production facility and the cell processing clean room at the present time is intended only for the production of Autologous VRX496-transduced CD4+ T cells. Appropriate change-over procedures are in place between vector production and subject cell transductions. All subject cell products are tracked throughout the production process by a bar code system (see below).

To minimize any possibility of cross-contamination, vector production and cell processing operations do not share personnel and the areas are physically separated by location. Control is additionally maintained through written standard operating procedures (SOP) and personnel training on these procedures.

The Vector Production Clean Room Suite and the Cell Processing Clean Room Suite both have been designed for cGMP production and biosafety containment: Both Clean Rooms are designed as Class 10,000 (ISO Class 7) and Biosafety Level 2 (BSL-2) large scale.

The Clean Rooms have separate Air Handling Units (AHU). The Vector Clean Room AHU is a constant-volume, recirculating unit containing supply and return fans; pre 45% filter and 95% final filter; cooling coils, and a DX condensing unit. The AHU for the Cell Processing Clean Room supplies 100% outside air. The clean room finishes are constructed of smooth, hard, cleanable water- and chemical-resistant surfaces and the floors of seamless vinyl flooring with integral cove. Doors are constructed of galvanized steel with safety glass vision panels. Hardware features on the doors include kick plates, mop plates and door closures.

Similarly to the movement within the production areas, the movement between Quality Control (QC) Laboratories is controlled by SOP. The DNA Extraction Lab (Q2) is also separated physically from the PCR Labs (Q5 and Q6) to minimize any risk of contamination.

### EXAMPLE 12

### The Cell Product Manufactured

The name of the exemplary cell product manufactured and described under this amendment is Autologous VRX496-transduced CD4+ T Cells.

VRX496 contains a 937-nucleotide antisense sequence targeted to the Human Immunodeficiency Virus (HIV) envelope gene.

Autologous VRX496-transduced cells are aliquoted into infusion bags (5×10⁹ to 10¹⁰ transduced cells per 90 ml bag). The cells are suspended in infusible cryomedia consisting of:

31.25% Plasmalyte-A,

31.25% Dextrose (5%),

0.45% Sodium Chloride,

7.5% Dimethylsulfoxide (DMSO),

1% Dextran 40, and

5% Human Serum Albumin.

### EXAMPLE 13

### Starting Materials

Described in the chart below is an exemplary list of starting materials used in the production of Autologous VRX496-transduced CD4+ T Cells.

| | | Source of Materials | | |
|---|---|---|---|---|
| | | (If of Human/Animal | | |
| Reagent | Description of Use | origin) | Reagent Quality | Vendor |
| Albumin (Human), | Excipient of infused | Human | U.S. Pharmacopeia/ | Abbott |
| USP | cell product | | U.S. FDA Licensed | |
| | | | Biologic | |
| CD4 Microbeads | Purification - | Humanized | Clinical Grade | Miltinyi |
| (Human) | Selection of | | | Biotech |
| | autologous T cells | | | |
| CD28 (9.3) | Cell Stimulation | Murine/Human | Clinical Grade | University of |
| Antibody | and Expansion | hybridoma | | Pennsylvania |
| CD3/CD28 | Cell Stimulation | Murine/Human | Clinical Grade | VIRxSYS |
| Conjugated | and Expansion | hybridoma | | |
| Magnetic Beads | | conjugated to | | |
| | | Dynal Magnetic | | |
| | | Microbeads | | |
| Dimethylsulfoxide | Cryomedia | Not applicable | Clinical Grade | Edwards |
| (DMSO) | component - | | | Lifesciences |
| (Cryoserv) | component of | | | |
| | infusible cell media | | | |
| Human Sera, Type | Cell growth and | Human | Clinical | Valley |
| AB | expansion | | | Biomedical |
| 10% LMD in 5% | Cryomedia | Not applicable | U.S. FDA | Abbott |
| Dextrose Injection | component - | | Approved Drug | |
| (Low molecular | component of | | | |
| weight Dextran for | infusible cell media | | | |
| intravenous | | | | |
| administration) | | | | |
| 5% Dextrose and | Cryomedia | Not applicable | U.S. FDA | Abbott |
| 0.45% Sodium | component - | | Approved Drug | |
| Chloride Injection, | component of | | | |
| USP | infusible cell media | | | |
| Immune Globulin | Purification - | Human | European | Grifols |
| Intravenous | nonspecific blocker | | Pharmacopeia; | |
| (Human) 5% | | | Clinical | |
| Interleukin-2 | Cell growth and | Human | U.S. FDA Licensed | Chiron |
| (Proleukin) | expansion | recombinant | Biologic | |
| Magnetic | Carrier for CD3 and | Not applicable | Clinical Grade | Dynal |
| Microbeads | CD8 purification | | | |
| | antibodies | | | |
| (Multiple | Included in Infusion | Not applicable | U.S. Pharmacopeia/ | Baxter |
| Electrolytes | to wash cells from | | U.S. FDA Licensed | |
| Injection, Type 1, | bag. Included to be | | Biologic | |
| USP)(Plasma-Lyte | used in case of an | | | |
| A Injection ph 7.4) | emergency | | | |
| Norvir (ritonavir | Inhibitor of HIV | Not Applicable | U.S. FDA | Abbott |
| oral solution) | protease in cell | | Approved Drug | Laboratories |
| | culture | | | |
| Oclone OKT3 | Cell Stimulation | Murine/Human | U.S. FDA Licensed | Ortho |
| Sterile Solution | and Expansion | hybridoma | Biologic | |
| (muromonrab- | | | | |
| CD3) | | | | |
| Phosphate | Physiological agent | Not Applicable | Clinical Grade | Baxter |
| Buffered Saline | for cells | | | |
| Solution | | | | |
| Recombinant | Enhancement of | Human | Clinical Grade | Takara Bio |
| Human Fibronectin | transformation | | | |
| Fragment | | | | |
| (RetroNectin) | | | | |
| Retrovir | Pyrimidine | Not Applicable | U.S. FDA | GlaxoSmith |
| (zidovudine) IV | nucleoside analogue | | Approved Drug | Kline |
| Infusion | against HIV in cell | | | |
| | culture | | | |
| Water for | | Not Applicable | U.S. FDA | Baxter, |
| Injection, USP | | | Approved Drug | Hospira Inc. |
| X VIVO-15 W/O | Cell growth and | Human | Clinical Grade | Cambrex |
| Gentamicin and | expansion media | | | |
| phenol red w/5% | | | | |
| Human Serum AB | | | | |
| VRX496 Lentiviral | Gene-transfer agent | Not Applicable | Clinical Grade | VIRxSYS |
| Vector | | | | |

All starting materials are received and inspected by Materials Management personnel. The inspection includes completing the approved “Raw Material Specification and Receiving Sheet.” A lot number is assigned, the package label examined and the Certificate of Analysis (C of A) reviewed for conformance to the approved “Raw Material Specification and Receiving Sheet.”

Quality Assurance (QA) reviews the “Raw Material Specification and Receiving Sheet” completed by Materials Management and either approves or rejects the material. If the material is “Approved” by QA it is labeled as “Approved” and moved by Materials Management personnel to an appropriate approved materials storage location within the Warehouse Area.

If a starting material is “Rejected” by QA, it is labeled as “Rejected” and segregated from the approved materials until a final disposition is determined, i.e., disposal, return to vendor, or transfer to R&D.

All starting materials are entered by QA into an Inventory Log. This log includes the Lot Number assigned, quantity received, disposition, and expiration. Included in this inventory are in-house formulations such as buffers used in the manufacturing process or in QC test procedures. A monthly list of materials, which expire at the end of the month is generated by QA for Materials Management and/or Production to ensure their removal from the area and prevent inadvertent use.

### EXAMPLE 14

### Production and Routine Controls

Flow Diagram of the Process

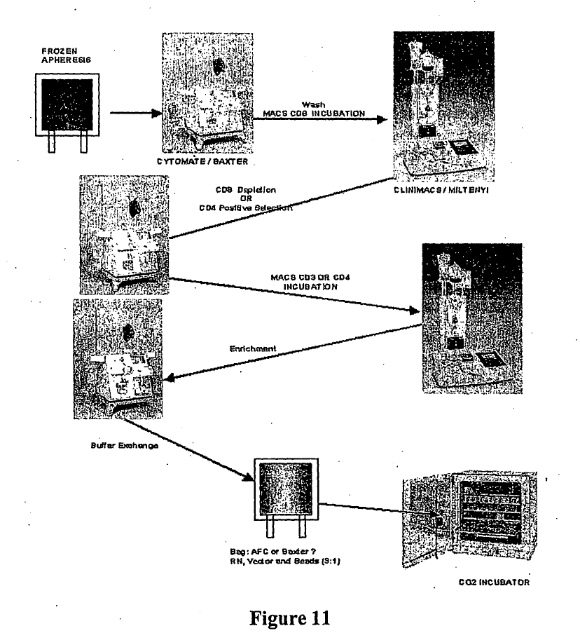

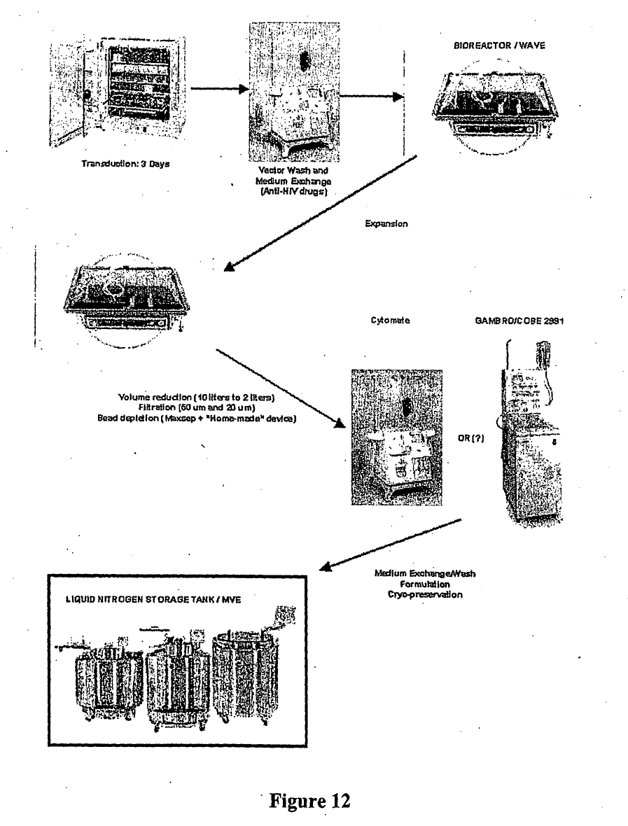

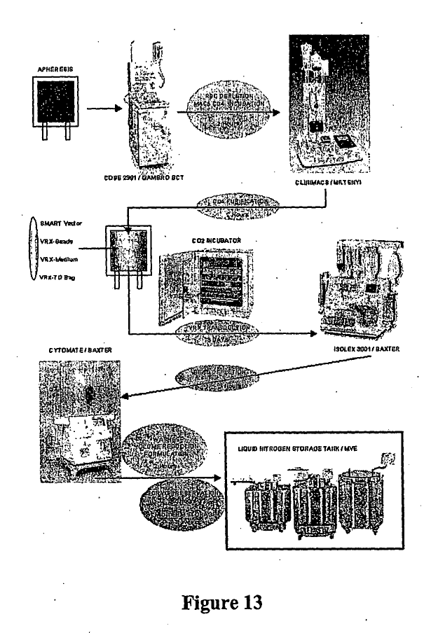

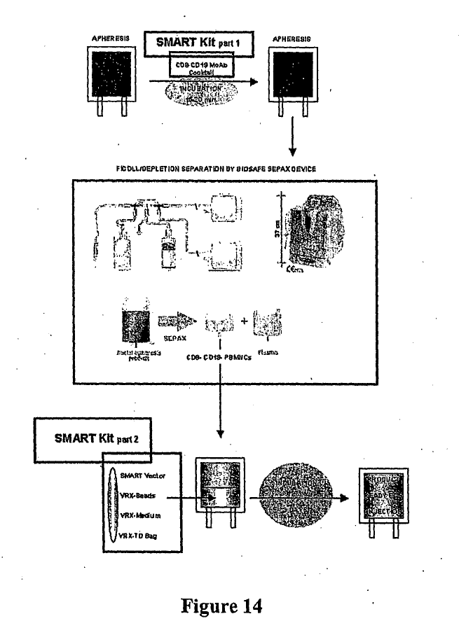

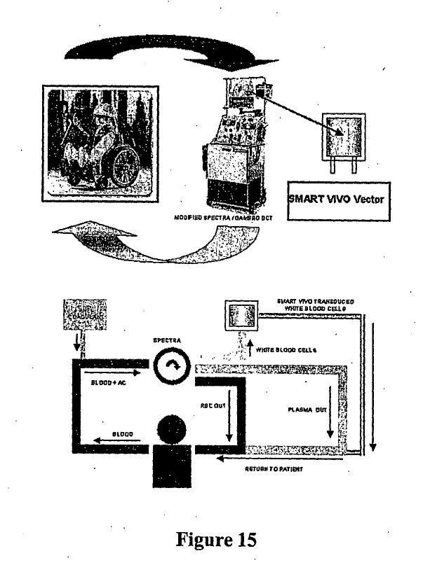

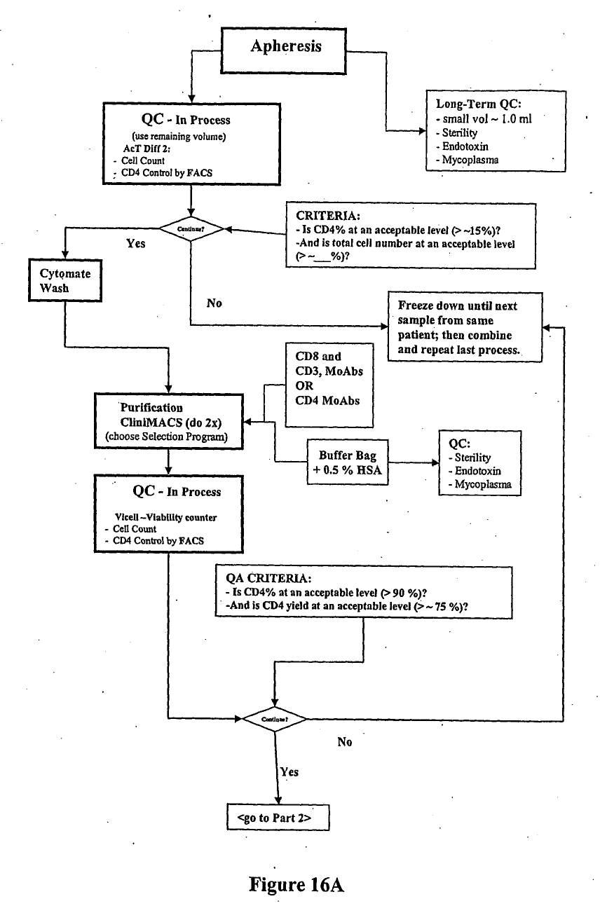

Attached is a diagram of the cell processing purification (FIG. 11) and manufacturing procedure (FIG. 12).

### EXAMPLE 15

### Description of the Process

Method of Cell Collection

The starting material for the production of Autologous VRX496-transduced CD4+ T Cells is peripheral blood mononuclear cells (PBMC). PBMC are obtained from an HIV-infected subject during leukapheresis. The leukapheresis procedure occurs in the blood collection facility using an automated cell separator (Cobe Spectra CS-3000, Baxter; Lakewood, Colo.).

Since up to eight cell infusions of approximately 5×10⁹ to 10 to 10¹⁰ autologous VRX496-transduced CD4+ T cells each may be given to the subjects during the Phase II clinical study, approximately 3 to 4 blood volumes (15 L) of blood is required to be processed through the Cobe Spectra to collect sufficient PBMC (approximately 10 to 20 billion) to undergo the cell washing and selection procedures (i.e., purification) to result in the requisite number of CD4 T cells (i.e., approximately 1 to 2 billion) to begin the cell transduction and expansion processes. A single leukapheresis procedure takes approximately 3 hours to complete.

In contrast, during Phase I clinical study, since each subject received only a single infusion of approximately 1×10¹⁰ autologous VRX496-transduced CD4+ T cells, the leukapheresed product collected was smaller, consisting of approximately 5 billion PBMC in approximately 70 mL.

The leukapheresed product will be shipped the day of its collection at ambient temperature from the respective blood center to wherever the product will be further processed, by an air or land transport courier in accordance with IATA and DOD regulations. Transit time will be planned to insure that the leukapheresed product is received for processing by production personnel no later than 24 hours after collection.

Since the product is both autologous and infectious, to insure product tracking control and to reduce the possibility of any product mix-up, each leukapheresed bag is labeled with:

A unique lot number,

The contained cell volume,

A unique bar code label,

The subject study ID (which includes identification of the study site),

The subject initials, and

The subject's birth date.

Additional precautions taken to reduce the possibility of mix-up are:

A written procedure allowing only 2 individual cell products to be manipulated at any given time within the cell processing clean room, and

Product manipulations must involve different stages in the production process (e.g., CD4+ T Cell selection, vector removal or cell harvest).

The exception to this policy is during the incubation and storage steps where as many as 80 separate products may be incubated in WAVE™ bioreactor and up to 30 stored in a freezer at any given time.

Tracking of cell product throughout the manufacturing process with the aid of a bar code system.

### EXAMPLE 16

### Receipt of Leukapheresed Product

Once the leukapheresed product is received at the cell processing facility, Quality Assurance (QA) performs barcode scanning in the receiving room along with verification of the product bag label and records:

Total volume received,

Lot number on the bag,

Time the bag was received, and

Number of hours from the time of leukapheresis to the time of receipt.

After QA releases the leukapheresed product, it is delivered by Materials Management personnel to the Cell Processing Clean Room (Class 10,000) (Biosafety Level 2 large scale). Production personnel take an approximate 5 cc sample of the cell product by syringe and add it to a vial for QC testing. QC tests for total CD4+ live cells, which should be ≧6×10⁸ cells.

### EXAMPLE 17

### Plasma Washing and MACS CD4 Incubation

The cells are washed to remove plasma and magnetically labeled (incubated) with CD4 microbeads (Miltenyi Biotech, Germany), which have been developed for the separation of human cells based on the expression of the CD4 antigen.

During Phase I clinical study, the starting material underwent ficoll density gradient separation by low speed centrifugation to remove plasma and then underwent COBE (Baxter) washing and resuspension in working buffer. The washed cell material was then incubated with CD8 high density microparticles (CD8-HDM nickel beads) (Biotransport) for subsequent magnetic separation using an Eligix Magnetic Cell Separation System.

For Phase II clinical study, cell washing to remove plasma will be performed using the CYTOMATE Cell Processing System (Miltinyi Biotech, Germany). The CYTOMATE Cell Processing System is a stand-alone, closed and automated device for washing and concentrating cellular products, and fluid transfer applications. It enables efficient cell washing with low cell loss and high viability. The system features a disposable tube set that creates a closed system fluid path for cell processing in a cGMP environment. It also makes fluid transfer flexible, fast and accurate. Solutions can be transferred to and from single or multiple containers, all within a closed system fluid path.

In addition, immune globulin solution (Immune Globubin Intravenous, USP, Grifols) will be added to prevent non-specific cell binding during incubation of the added CD4+ microbeads (Miltinyi Biotech).

The end product bag (CD4 microbead incubated cell suspension) is heat sealed and the bag is removed and placed under the biological safety hood. A QC sample of about 5 cc is taken for performing:

Cell concentration and

FACS analysis to determine the percentage of CD3+ CD8+ and the percentage of CD3+ CD4+.

Production stops until the QC results are received (approximately 30 minutes). The volume of the CytoMate end-product volume is calculated.

### EXAMPLE 18

### CD4+ Selection

As noted above, during Phase I clinical study, an Eligix™ Cell Separation System was used for CD8 depletion. For Phase II clinical study, CD4+ positive selection will be performed via a CliniMACS magnetic cell separation system. This system uses a sterile CliniMACS disposable set consisting of (1) a transfer pack container, (2) plasma transfer sets with female luer adapters for connection to a buffer bag and a cell suspension bag and (3) plasma transfer sets with female luer adapters for connection to a positive selection bag and a waste collection bag.

Spiking of the sterile disposal sets' buffer and cell suspension lines to the respective bags are done under a biological safety hood to maintain sterility.

Once the phosphate buffered saline (PBS) buffer and cell suspension lines have been spiked, the disposable set is attached to the CliniMACS and the CD4 magnetic labeled cell suspension is run through the CliniMACS. The collected positive fraction will be used to continue with the process.

The rationale for changing from a 2 step process during Phase I clinical study, i.e., CD8 depletion and CD3 selection, to a 1 step process during Phase II clinical study, i.e., CD4 selection, is for cell processing efficiency and to obtain a purer cell product.

### EXAMPLE 19

### Buffer to Media Exchange

PBS to X-VIVO-15 media (Cambrex; Walkersville, Md.) exchange is accomplished via the CytoMate. The end product bag is removed, heat sealed and placed under the biological safety hood. A transfer set with female luer adapter is attached to the product bag and a 5 cc sample is obtained by syringe for QC testing for:

The percentage of CD3+ CD8+ cells and CD3+ CD4+ cells,

Cell Viability,

Cell Number, and

Pre-expansion GIV Gag measurement.

Cell production stops until QC results are obtained. If the cells meet specification, production continues with cell transduction.

### EXAMPLE 20

### CD4+ T Cell Transduction

CD3/CD28 co-stimulation beads (Dynal beads, Oslo, Norway, coated with anti-CD3 (OKT3) and anti-CD28 (UPenn monoclonal antibody 9.3) are added to the CD4+ T Cell suspension followed by the addition of the VRX496 viral vector product. The whole mixture of CD4+ T cells, X-VIVO+5% Human Serum Albumin, IL2, NAC, CD3/CD28 microbeads and VRX496 vector suspension (5% W/V) are added to a Nunc™ cell factory coated with RetroNectin (Takara Bio, Japan) and the cell factory put into a humidified, 37° C., 5% CO₂ incubator. VRX496 vector suspension (5% W/V) is once again added the next day. The cells are incubated with vector for 3 days, then transferred to WAVE™ cell bag and placed into a Wave™ Bioreactor (WAVE™ Biotech LLC, Bridgewater, N.J.).

The WAVE bioreactor has a special rocking platform. The rocking motion of this platform induces waves in the culture fluid. These waves provide mixing and oxygen transfer, resulting in a perfect environment for cell growth that can easily support over 20×10⁶ cells/ml. Tubing leads on the bags and a variety of connecting devices (connection will be via spike connectors and welds produced via the Terumo Sterile Connecting Device) allow the cells to be grown in a closed system with minimal risk of contamination.

### EXAMPLE 21

### Washing to Remove Vector

On the 4^{th} day, the cells are washed 2 times with X-VIVO 15 using the CytoMate cell washer.

### EXAMPLE 22

### Cell Expansion

The cultures are maintained for 7 to 12 days until it is time to harvest them. The cells are counted at least every other day and fresh medium is added to maintain the cells at an approximate density of 0.5-1.5×10⁶ cells/ml. Antiretroviral drugs (Norvir, Abbot Laboratories, and Retrovir, GlaxoSmithKline) (1 μmol/L) are added to inhibit HIV replication while the cells are in culture. At about day 10, the cells are ready for harvesting. A post-expansion HIV Gag measurement is performed to insure that the post-expansion HIV copies are not greater than pre-expansion HIV copies. From the pre-harvest cells, a sample is taken to test for mycoplasma.

### EXAMPLE 23

### Washing, Volume Reduction and Formulation

The bag of cells are loaded on the CytoMate and the cells are washed out of the nutrient media and into an infusible cyromedia solution consisting of:

31.25% PlasmaLyte A,

31.25% Dextrose 5%,

0.45% NaCl,

5% Human Serum Albumin (HSA),

1% Dextran 40, and

7.5% DMSO.

### EXAMPLE 24

### CD3/CD28 Microbead Depletion

The CD3/CD28 microbeads are removed by passing the culture bag over a MaxSep™ magnet (Baxter). The beads are retained on the magnets and the cells are poured into another bag. The cells are assayed for residual beads.

### EXAMPLE 25

### Cryopreservation

The VRX496-transduced CD4+ T Cells are controlled-rate frozen. The cells cooling the product to cool at one degree (1° C.) per minute until the product reaches the point of phase transition; then the freezing rate is increased until the temperature reaches −90° C.

### EXAMPLE 26

### Quality Control (QC) Release Testing

Samples are taken for QC Release Testing. The cell product is stored in the vapor phase of a liquid nitrogen freezer (set point of <−130° C.) until completion of the QC testing.

### EXAMPLE 27

### Quality Assurance (QA) Release

After completion of QC testing, QA reviews all test results and if the release specifications have been met, authorizes the release of cell product for use in the clinical trial.

### EXAMPLE 28

### Storage

QA released cell product remains in liquid nitrogen storage until it is ready for shipping to the clinical site.

### EXAMPLE 29

### Shipping to Clinical Sites

Cell product is shipped to clinical sites at a temperature of ≦140° C. in liquid nitrogen vapor shippers (Chart Inc., Marietta, Georgia, formerly MVE Cryogenics) by contract transport courier (Cavalier Logistics Management, Inc., Dulles, Virginia) by their own freight truck or by commercial airline(s). These cryo-shippers have been validated to maintain their charge for 8 days.

### EXAMPLE 30

### Quality Control (QC) Testing

QC In-Process Testing

In-process QC testing is performed as shown below. At this stage of development, these tests are done for information only.

| Process | | | |
|---|---|---|---|
| Step | Description | Test for | Test Article |
| 2 | Apheresed Product | Cell | PBMC |
| | | Concentration | |
| | | % CD4 | |
| | | % CD8 | |
| 5 | Positive Selected | Cell | CD4+ T Cells |
| | CD4+ T Cells | Concentration | |
| | | % CD4 | |
| | | % CD8 | |
| | | Viability | |
| | | Pre-HIV gag | |
| 8 | Cell Expansion | Cell Count | CD4+ T Cells |

### EXAMPLE 31

### QC Release Testing

The final cell product release tests and specifications are shown in the Certificate of Analysis. The release tests are performed at the process steps and on the test articles presented below.

| Process | | | |
|---|---|---|---|
| Step | Description | Test for | Test Article |
| 4 | Positive | Pre-expansion HIV | Transduced |
| | Selected | gag to compare | Cells |
| | Cell Product | to post-expansion | |
| | | gag in step 10 | |
| 10 | Pre- | Mycoplasma | Culture |
| | harvested Cell | | Supernatant |
| | Product | Bovine Serum | Culture |
| | | Albumin (BSA) | Supernatant |
| 10 | Harvested | Microbead Removal | Transduced |
| | Cell | | Cells |
| | Product | | |
| 10 | Post- | Gtag | Transduced |
| | Harvest Cell | E1A | Cells |
| | Product | Post-expansion | |
| | | HIV gag | |
| | | VSVg RNA | Wash |
| | | | Supernatant |
| 11 | Cryo- | Sterility | Transduced |
| | preserved | | Cells |
| | Product | Endotoxin | Transduced |
| | | | Cells |
| 1-Day Prior | Infused | Viability | Transduced |
| to Dosing | Cell Product | | Cells |

### EXAMPLE 32

### Qualification of the Production Process

### Summary of Major Manufacturing Changes Made Between Phase I and Phase II

Table 1 presents a summary of the major manufacturing changes made between Phase I and Phase II.

TABLE 1
Summary of Major Manufacturing Changes
Made Between Phase I and Phase II

| Manufacturing Change | Description |
|---|---|
| Initial Wash of | From ficoll wash to CytoMate wash |
| Apheresed Cell | |
| Product to | |
| Remove Plasma | |
| CD4 Purification | Changed from CD8 depletion (Eligix) (CD8 |
| Process | antibody conjugated to nickel HDM) to CD4 |
| | selection (Miltinyi) (CD4 antibody |
| | conjugated to iron microbeads) |
| Cell Washing | Changed from washes using Cobe (Baxter) to |
| throughout Process | washes using Cytomate (Miltinyi) |
| CD3/CD28 co- | Presently produced by VIRxSYS using the same |
| stimulation | antibodies and beads used by the University of |
| microbeads | Pennsylvania Vaccine and Cell Production |
| | Facility for Phase I clinical study |
| Transduction | Performed in a cell factory rather than |
| | plastic bags on 2 days each with 5% |
| | suspension (W/V) of vector rather than 10% |
| | suspension (W/V) of the vector. |
| Cell Expansion | Presently incubating cells with WAVE incubator |

### EXAMPLE 33

### Quality of the Apheresed Cell Product and Post-Positive Selected Cell Product: Phase I Versus Phase II

Table 2 provides a comparison of the CD4+ T Cell purity of the Phase I and Phase II starting material (i.e., post-washed apheresed product) and the post-positive selected cell product (i.e., cell product used to start the CD4+ T cell transduction and cell expansion).

As can be seen from these data, the Phase II cell production process results in a purer CD4+ starting material (average of 28.04% CD4 versus 14.58% CD4 for Phase I) and a purer CD4+ cell product to start the VRX496 transduction and cell expansion (average of 95.60% CD4 versus 36.82% CD4 for Phase I.

Additionally, the data show that the Phase II production process results in a post-positive selection cell product that is more consistent in purity than the product used in Phase I clinical study. This can be attributed to the single CD4+ positive selection step, whereas, the Phase I process used a 2-step process: CD8+ depletion and CD3+/CD4+ positive selection.

TABLE 2
Comparison of CD4+ T Cell Purity: Phase I
Cell Product Versus Phase II Development Lots

| | Apheresed Product | | Post-Positive Selection | |
|---|---|---|---|---|
| | | VIRxSYS Phase II | | VIRxSYS Phase II |
| | UPenn Phase I | Development Lots | UPenn Phase I | Development Lots |
| | Cell Product | Post-CytoMate Wash | Cell Product | Post-CytoMate Wash/ |
| Lot # | Post-Wash | and CD4+ Incubation | Post COBE Wash | Media Exchange |
| 1 | 11% | 38.93% | 56% | 97.62% CD4+ purity |
| | Abs. 6.93 × 10⁹ | Abs. 6.124 × 10⁹ | Abs. 3.06 × 10⁹ | Abs. 3.419 × 10⁹ |
| | | | (52% recovery) | (47.7% recovery) |
| 2 | 15.9% | 20.30% | 52.2% | 97.4% CD4+ purity |
| | Abs. 1.595 × 10⁹ | Abs. 3.00 × 10⁹ | Abs. 4.38 × 10⁸ | Abs. 1.48 × 10⁹ |
| | | | (48% recovery) | (40.5% recovery) |
| 3 | 10.7% | 24.9% | 23% | 91.77% CD4+ purity |
| | Abs. 1.015 × 10⁹ | Abs. 3.71 × 10⁹ | Abs. 1.67 × 10⁸ | Abs. 2.05 × 10⁹ |
| | | | (26% recovery) | (48.8% recovery) |
| 4 | 25.9% | None | 33.4% | None |
| | Abs. 2.533 × 10⁹ | | Abs. 2.93 × 10⁸ | |
| | | | (30% recovery) | |
| 5 | 9.4% | None | 19.5% | None |
| | Abs. 7.86 × 10⁸ | | Abs. 1.77 × 10⁸ | |
| | | | (23% recovery) | |
| Avg. % | 14.58% | 28.04% | 36.82 | 95.60% |
| CD4: | (range 9.4-25.9%) | (range 20.3-38.93%) | (range 19.5-56%) | (range 91.77-97.62%) |

TABLE 3
Comparison of Phase I and Phase II Processes:
CD4+ T Cell Expansion

| | Phase I Cell Process | | Phase II Cell Process | |
|---|---|---|---|---|
| | (Clinical Trial Subject Lots) | | (Development Lots) | |
| | # of Cells at | | # of Cells at | |
| | End-of-Culture | Fold | End-of-Culture | Fold |
| Lot # | (×106 cells) | Expansion | (×106 cells) | Expansion |
| 1 | 15,811 | 65 | 52,296 | 28.6 |
| 2 | 20,638 | 40 | 104,000 | 58.8 |
| 3 | 6,785 | 25 | 96,646 | 63.0 |
| 4 | 11,454 | 32 | | |
| 5 | 15,212 | 66 | | |
| Avg | 13,980 | 58.8 | 84,314 | 50.1 |

### EXAMPLE 34

### Comparison of VRX496 Transduction Efficiency

### Phase I Versus Phase II

Table 4 presents a comparison of the VRX496 average vector copy number per cell between the Phase I cell products and the Phase II Development lots. As can be seen, average vector copy number remains essentially unchanged from Phase I, however, the average vector copy number among the Phase II Development lots are more consistent than those in Phase I.

TABLE 4
Comparison of Transduction Efficiency (Average VRX496
Vector Copy Number per Cell): Phase I Subject's
Cells Versus Phase II Process Development Lots

| Phase I Cell Product | | Phase II Development Lots | |
|---|---|---|---|
| Subject Study | Results for Final | | Results for Final |
| ID | Cell Product | Process Run # | Cell Product |
| 001-022 J-K | 1.20 | 1 | 2.80 |
| 001-017 A-J | 4.10 | 2 | 1.19 |
| 001-010 RAG | 0.98 | 3 | 1.48 |
| 0001-001 JFJ | 1.80 | | |
| 001-002 R-B | 2.3 | | |
| Avg. | 2.08 | | 1.82 |
| Range | 0.98 to 4.10 | | 1.19 to 2.80 |

Specification = average of 0.5 to 5.0 VRX496 copies per cell.

### EXAMPLE 35

### Summary of Release Test Results for Phase II Development Lots

Table 5 presents a summary of the release test results for Phase II Development Lots 1, 2 and 3. All three development lots have met lot release specifications.

TABLE 5
Summary of Release Test Results for Phase II Development

| | | Develop- | Develop- | Develop- |
|---|---|---|---|---|
| | | ment | ment | ment |
| Release Test | Specification | Lot #1 | Lot #2 | Lot #3 |
| Vector Copy# | 0.5-5.0 | 2.8 | 1.19 | 1.48 |
| Viability | ≧70% | 83.6 | 73.6 | 70.5 |
| VSVg DNA | Mo copies | 0 | 0 | 0 |
| BSA | | | | |
| E1A | | | | |
| HIVgag | | | | |
| Sterility | | | | |
| Mycoplasma | Not Detectable | Pass | Pass | Pass |
| Endotoxin | <3.5 EU/ml | 0.06 | 0.06 | 0.06 |
| Residual beads | <100 per | 0 | 0 | 10 |
| | 3 × 10⁶ cells | | | |
| RCL | | | | |

### EXAMPLE 36

### Stability of the Cell Product

The VRX496-transduced CD4+ T cell products manufactured for the Phase I clinical trial were cryopreserved and stored at ≦−80° C. until scheduled for subject infusion. On the day prior to infusion, a sentinel vial sample of cell product was thawed and measured for cell viability as part of the cell product release criteria. Each of the Phase I manufactured cell products had a cell viability of ≧70%. The longest time period of ≦−80° C. storage was 6 months. These data are supportive of the stability of the autologous VRX496-transduced cell product when stored at ≦−80° C. for up to 6 months.

To assess stability, a 24-months stability study of autologous VRX496-transduced CD4+ T cells will be performed on 6 autologous VRX496-transduced CD4+ T cell lots. These lots will be transduced with 2 different lots of VRX496 vector manufactured according to the existing manufacturing plan (i.e., 3 VRX496-transduced cell product lots per vector lot). The storage condition will be liquid nitrogen. Transduced cell product samples (15 ml) will be assayed at 3, 6, 12, 18 and 24 months. Time 0 data will be transduced cell product lot release data. Sufficient samples (20 bags per lot) will be collected at the end-of-cell processing to use for assaying at each time point. Assays will include: Appearance, Gtag copy number, cell viability, recovery, Intra-Cellular cytokine staining, sterility, and extra-cellular DNA concentrations. Interim reports will be written at the completion of testing for each time interval. A final report will be written at the end of the study. The QA department will be responsible for assuring the integrity of the data generated and for ensuring compliance with cGMP. All raw data, records and reports generated will be maintained at the corporation. Records to be maintained will include storage conditions, storage unit validation and maintenance, sample preparation and raw assay data.

### EXAMPLE 37

### Autologous Cell Product Tracking Procedures

Autologous CD4+ T cells for 4 different subjects may be processed concurrently. To protect these cell products from potential mix-up and contamination during this concurrent manufacturing, these 4 cell products will be processed during different stages of production. Current good manufacturing practices will be followed. There are approved written cell processing procedures and all production personnel receive training on these procedures. Dedicated production equipment is used with procedures for production lot change-over. Critical equipment (incubator, freezers, HVAC) have been validated. Water for processing and all production materials used are obtained from approved vendors and according to established specifications. The following special controls to track subject cell products throughout the cell processing procedure have also been implemented:

### EXAMPLE 38

### Barcode System

A custom designed barcode system tracks subject cells throughout the cell production and QC testing process, i.e., receipt, cell transduction, expansion, cryopreservation, storage, packaging and shipping.

The barcode system provides an audit trail, user level access and full reporting capabilities.

Prior to a subject cell product being processed or tested, production personnel scan both the material being processed or tested and the barcode affixed to the batch production records or Quality Control (QC) test document for an identical match. If these do not match, a warning is given on the computer screen. The individual scanning the material must then attest that a reconciliation was made and initials and dates the batch production records or QC test document.

### EXAMPLE 39

### Documentation

Each subject's cell product lot is assigned a different color of documentation (i.e., unique color for batch production records and QC test documents) to visually separate subject cell products during processing.

### EXAMPLE 40

### Segregation and Controls of Cell Product During Processing

Only one production person is authorized to work with one subject cell product at any given time and all operations involving this cell product must be concluded before the next subject's cell product can be processed.

All open air cell product manipulations are performed in a Class 100 Biological Safety Hood. Only cells from one subject are manipulated in the hood at any time.

Subject cell product is incubated in WAVE™ bags and each subject's cell product lot has its own dedicated WAVE™ Incubator.

Raw Materials such as buffers and reagents, which are placed into the hood are dedicated to one subject's cell product lot and discarded at the end of processing.

### EXAMPLE 41

### Segregation and Accountability of Cell Product During Storage

Only one subject's cell product is cryopreserved at one time.

During cryopreservation, each subject's cell product bags are protected within metal cassettes. After cryopreservation, these cassettes are connected by cable ties and stored segregated in freezer racks.

Inventory of all stored cell product is maintained in the barcode system and by hardcopy documents.

### EXAMPLE 42

### Overview of Current Process

The proposed cell processing procedures for the up-coming Phase I/II clinical trials in the facility are summarized below. Briefly, the apheresis product will first go through a red blood cell (RBC) depletion using the COBE 2991 cell processor (Gambro BCT). The resulting product will be then incubated with the Miltenyi anti-CD4 MACS and washed with the COBE 2991 cell processor. The anti-CD4 incubated product will be processed on the CliniMACS device, likely run twice for maximal yield.

The CD4 selected product will be immediately transduced with the vector in presence of stimulating beads in a RetroNectin coated bag. Transduction will be carried out for three days in a 37° C.-5% CO₂ incubator. Post-transduction cells will be washed using the Cytomate device (Baxter Oncology) before being expanded for a period of 8 to 10 days in the Wave Bioreactor. After expansion, stimulating beads will be removed using the Isolex 300i or Maxsep (Baxter Oncology, both), cell culture volume will be reduced, and cells washed using again the Cytomate, and prepared for cryo-preservation (formulation). Cryo-preservation will done with the Cryo-Med control rate freezer and cells will be stored in a vapor phase liquid nitrogen MVE tank. Overall, the process should take 11 to 13 days.

As proposed, the current cell processing procedures are time consuming, and expensive, but could be quickly implemented. The major cost identified in this process is the antibody selection step, and the major limitation for processing large number of subjects is the 8 to 10 days expansion step.

Below are presented some technical alternatives aimed to simplify the current cell processing procedures, starting from the easiest to implement.

The first technical alternative concerns the length of expansion step, reducing it from 8 to 10 days to 0. Briefly, 3 day transduced cells will be directly processed for cryo-preservation (bead depletion, washes, and formulation). By reducing the time of product preparation from 11 to 13 days to 3 days, this will allow to process more product during the same period (4 vs. 1) and reduces as well associated expansion cost (Wave bioreactor and culture medium).

Associated with this first alternative, a limited or no purification step could be implemented, thus reducing the associated purification cost.

The second alternative would be the creation of a transduction kit, simple enough to be used at the clinical site without excessive and time-consuming manipulation procedures. Briefly the fresh apheresis product will be directly incubated with a cocktail of antibodies that will link the RBC to unwanted cells, such as CD8+ and CD19+ lymphocyte (RosetteSep product, from Stemcell Technologies). Using the compact, automated and closed Sepax device (Biosafe), unwanted cells will sediment with the RBC during the centrifugation over a ficoll layer. Mononuclear cells will be collected, washed off ficoll with the same device, and transferred to a Teflon® bag already containing the vector and the stimulating biodegradable nanobeads. Transduction will be carried for 3 days in a 37° C.-5% CO₂ incubator, before washed using again the Sepax device and being immediately re-injected to the subject.

The third alternative is summarized in FIG. 15. This technical alternative is using only one processing step, the apheresis procedure, and is done at the clinical site in few hours. Briefly, the subject undergoes an apheresis procedure the same way he goes for the current and other proposed alternatives. The concentrated white blood cells are normally collected into a bag while RBC and plasma are continually re-infused to the subject. The collected white blood cells are then transduced in the collecting bag before being re-infused to the subject. No ex vivo manipulations are required.

### EXAMPLE 43

### Isolation

Doing just RBC depletion is the cheapest alternative; no clinical grade antibodies are required. The RBC depletion is not time consuming, and requires only one piece of equipment. However there is no control regarding the CD4 content.

The limited CD8 CD19 depletion alternative with the Sepax device needs only one piece of equipment, but might be more expansive due to the number of clinical antibodies required for the depletion procedure (3) and the licensing of the Stemcell Technologies IP.

The current isolation procedure, CD4 positive selection, requires two pieces of equipment to be performed, and one clinical grade antibody (soon commercially available). The cost might be similar to the limited selection, but this procedure is more time consuming. However, the CD4 content is well controlled.

It was demonstrated one year ago that cells that went under only CD14 depletion, or CD14 and CD8 depletion, or CD14 depletion and CD4 purification had similar transduction levels, as assayed by flow cytometry. However the difference was in the level of expansion after a 7 days culture period.

### EXAMPLE 44

### Expansion

The current process has a culture period of 8 to 10 days.

The proposed alternative is to reduce the time of expansion to the minimum necessary for the transduction. However few, if any, data are currently available to assess the in vivo expansion potential of 3 days manipulated T cells. Furthermore, the lack of an appropriate small animal model to assess the T cells reconstitution is a major limitation.

One proposed way to achieve in vivo expansion of transduced T cells would be to select them using the MGMT approach. Brian Davis, et al., demonstrated two years ago it was possible in vitro to select transduced primary CD4 T cells from 5% to over 80% using BG/BCNU drug treatment. Again, the lack of an appropriate animal model to assess the exact in vivo drug dosing is a major limitation.

Another option is the subject (in vivo) pre-conditioning with an anti-CD3 antibody before re-infusion of the manipulated cells. In vivo T cell depletion before transduced cell re-infusion could lead to a quick reconstitution of the T cell subset with the re-injected cells. Evaluation in large animal model or directly in Phase I clinical trial seems to be the most appropriate way to go.

### EXAMPLE 45

### Stimulation

The current stimulation procedure uses anti-human CD3 and anti-human CD28 murine antibodies coated to Dynal epoxy beads. CD3/CD28 stimulation is a feature of the cell transduction protocol.

Four alternative embodiments are described below:

Using CD3 and CD28 antibodies linked on biodegradable nanobeads. This approach will not reduce the associated antibody stimulation cost but will reduce the product manipulation (no bead removal).

Using a superagonist anti-human CD28. This antibody has been shown to efficiently stimulate T cell expansion without the need of an anti-CD3 antibody.

Using the vector itself as T cell stimulatory proteins carrier. This approach does not require antibody production but will require modification for the packaging cell line.

Using the Tetralink system developed by Stemcell Technologies. This system bypass the need of bead support, thus avoiding the bead depletion step. This system requires the use of murine IgG1 monoclonal antibodies to be functional.

### EXAMPLE 46

### Kit

One embodiment of the invention is a kit, including clinical grade vector produced from modified packaging cell line using biodegradable nanobead free stimulating system, and safety/efficacy from large animal model for 3 days culture period and in vivo T cell reconstitution.

Having now fully described this invention, it will be appreciated by those skilled in the art that the same can be performed within a wide range of equivalent parameters, concentrations, and conditions without departing from the spirit and scope of the invention and without undue experimentation.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

Hereinabove and in the claims below, use of the terms “a” or “an” is not limited to defining the singular state. Instead, use of the terms encompasses the plural state. For example the term “an antibody” is not limited to the singular state of one single antibody molecule, but rather encompasses the presence of a plurality of antibody molecules so long as they are identical copies of the antibody being referred to. Similarly, “a viral vector” is not limited to one single viral vector molecule or one single viral particle. The term “or” is not meant to be exclusive to one or the terms it designates. For example, as it is used in a phrase of the structure “A or B” may denote A alone, B alone, or both A and B.

1. A method for stable transduction of primary cells of the hematopoietic system and/or hematopoietic stem cells, comprising contacting, in vitro or ex vivo, the surface of the cells with both a lentiviral vector and at least one molecule which binds the cell surface, and culturing the cells in a ventilated vessel comprising two or more layers under conditions conducive to growth and/or proliferation, wherein the vessel is suitable for culturing at least about 100 million cells.

2. A method for stable transduction of primary cells of the hematopoietic system and/or hematopoietic stem cells, comprising contacting, in vitro or ex vivo, the surface of the cells with both a lentiviral vector and at least one molecule which binds the cell surface, and culturing the cells in a ventilated vessel comprising two or more layers under conditions conducive to growth and/or proliferation, wherein the vessel is suitable for culturing at least about 100 million cells; and
wherein the cells are transduced with the lentiviral lentiviral vector at a multiplicity of infection (MOI) such that the copies of lentiviral lentiviral vector per transduced cell is from about 0.5 to about 10; and
wherein contacting the cells with a lentiviral vector is for about 24 hours and is optionally repeated at least once.

3. A method for stable transduction of primary cells of the hematopoietic system and/or hematopoietic stem cells, comprising contacting, in vitro or ex vivo, the surface of the cells with both a lentiviral vector and at least one molecule which binds the cell surface, and culturing the cells in a ventilated vessel comprising two or more layers under conditions conducive to growth and/or proliferation, wherein the vessel is suitable for culturing at least about 100 million cell; and
wherein at least about 50% of the cells are stably transduced after about seven to ten days, or at about 14 days; and optionally at least 50% of the cells remain stably transduced after about 14 days; or
wherein at least about 75% of the cells are stably transduced after about seven to ten days, or at about 14 days, and optionally at least 75% of the cells remain stably transduced after about 14 days; or
wherein greater than 80%, 85%, 89%, 90%, 91%, 92%, 93%, 94% or 95% of the cells are stably transduced after about 14 days; or
wherein the cells are transduced with the lentiviral vector at a multiplicity of infection (MOI) of from about 2 to about 50, or from about 10 to about 30, or from at 10, or at about 20, or at about 30, or at about 40, or at about 50, or from about 1 to about 400, or less than 500; or
wherein the cells are transduced with the lentiviral vector at a multiplicity of infection (MOI) such that the copies of lentiviral vector per transduced cell is from about 1 to about 100; or
wherein the cells are transduced with the lentiviral vector at a multiplicity of infection (MOI) such that the copies of lentiviral vector per transduced cell is from about 0.5 to about 10; or
wherein contacting the cells with a lentiviral vector is for about 24 hours and is optionally repeated at least once; and
wherein the cell surface molecule does not induce apoptosis and the cell surface binding molecule results in the cell being more receptive to transduction by a viral lentiviral vector.

4. The method of claim 1, wherein the primary cells are isolated or derived from a subject.

5. The method of claim 4, wherein the primary cells are isolated by one or more of the following procedures:
(a) by apheresis of a subject's blood; or
(b) from bone marrow from a subject's bone; or
(c) by apheresis of an allogeneic subject's blood; or
(d) from bone marrow from an allogeneic subject's bone.

6. The method of claim 4, wherein the subject is infected with a human immunodeficiency virus (HIV), wherein optionally the HIV is HIV-1 or HIV-2.

7. The method of claim 4, wherein the subject has cancer, wherein optionally the cancer is breast cancer.

8. The method of claim 4, wherein the subject is a human or an animal.

9. The method of claim 1, wherein the primary cells are enriched prior to contact with the lentiviral vector or cell surface binding molecule by passing the cells over a gradient density buffer and/or by immuno-purification over a magnetic field.

10. The method of claim 1, wherein the contacting of the primary cells:
(a) with the lentiviral vector occurs before contacting the cells with at least one cell surface binding molecule; or
(b) with the lentiviral vector occurs simultaneously with contacting the cells with at least one cell surface binding molecule; or
(c) with the lentiviral vector occurs after contacting the cells with at least one cell surface binding molecule; or
(d) with the lentiviral vector occurs more than once; or
(e) with the lentiviral vector occurs continuously, after the simultaneous contacting of the cells with the lentiviral vector and the at least one cell surface binding molecule; or
(f) with cell surface binding molecule occurs continuously, after the simultaneous contacting of the cells with the lentiviral vector and the at least one cell surface binding molecule; or
(g) with the lentiviral vector and the at least one cell surface binding molecule occurs continuously, after the initial simultaneous contact of the cells with the lentivirus vector and the at least one cell surface binding molecule; or
(h) wherein any of (a) through (g) occurs at least once over a time period of about 24-36 hours.

11. The method of claim 1, wherein the primary cells are pre-stimulated with at least one cell surface binding molecule, and optionally the cells are pre-stimulated with the at least one cell surface binding molecule within about twenty four (24) hours prior to simultaneously contacting the cells with the lentiviral vector and the at least one cell surface binding molecule, or and optionally the cells are pre-stimulated with the at least one cell surface binding molecule within about 12 to 96 hours prior to simultaneously contacting the cells with the lentiviral vector and the at least one cell surface binding molecule.

12. The method of claim 1, wherein the lentiviral vector comprises at least one cis-acting nucleotide sequence derived from the gag, pol, env, vif, vpr, vpu, tat or rev genes, and optionally, wherein the sequence is not expressed or is a fragment or a mutant of the gag, pol, env, vif, vpr, vpu, tat or rev genes.

13. The method of claim 1, wherein the lentiviral vector is:
(a) pseudotyped and optionally wherein the pseudotyped vector contains the vesicular stomatitis virus G envelope protein; or
(b) pseudotyped, and wherein the pseudotyping comprises co-transfecting or co-infecting a packaging cell with both the lentiviral vector genetic material and genetic material encoding at least one envelope protein of another virus or a cell surface molecule; or
(c) pseudotyped with a Rhabdovirus, and optionally wherein the Rhabdovirus is a Vesicular Stomatitis Virus envelope G (VSV-G) protein.

14. The method of claim 1, wherein the primary cell is a lymphocyte, a precursor of a lymphocyte, a CD4 positive cell, a hematopoietic stem cell of a CD4 positive cell, a CD8 positive cell, a hematopoietic stem cell of a CD8 positive cell, a CD34 positive cell, a hematopoietic stem cell of a CD34 positive cell, a dendritic cell, a cell capable of differentiating into a dendritic cell, a human primary cell of the hematopoietic system and/or a human hematopoietic stem cell, a precursor of a human hematopoietic stem cell, an astrocyte, a skin fibroblast, a epithelial cell, a neuron, a dendritic cell, a leukocyte, a cell associated with the immune response, a vascular endothelial cell, a tumor cell, a tumor vascular endothelial cell, a liver cell, a lung cell, a bone marrow cell, an antigen presenting cell, a stromal cell, an adipocyte, a muscle cell, a pancreatic cell, a kidney cell, an ovum, a spermatocyte, a cell that contributes to the germ line, an embryonic pluripotential stem cell or a progenitor cell, a blood cell, a non-nucleated cell, a platelet cell, or an erythrocyte, or a derivative thereof.

15. The method of claim 1, wherein the at least one cell surface binding molecule:
(a) comprises a polypeptide, a lipid, a nucleic acid, a carbohydrate or an ion; or
(b) comprises an antibody, an antigen binding fragment, a ligand, or a cell surface molecule; or
(c) comprises FLT-3 ligand, TPO ligand, or Kit ligand, or a polypeptide or other binding molecule that is a cell surface binding analog of FLT-3 ligand, TPO ligand, or Kit ligand; or
(d) comprises CD34, CD3 ligand, CD28 ligand, CD25 ligand, CD71 ligand, or CD69 ligand, or a polypeptide or other binding molecule that has the same cell surface binding specificity of CD34, CD3, CD25, CD28, CD69 or CD71 ligand; or
(e) comprises a composition comprising GM-CSF, IL-4, and TNF-alpha; GM-CSF and interferon-alpha; or a polypeptide or other binding molecule that is a cell surface binding analog of GM-CSF, IL-4, and TNF-alpha; GM-CSF or interferon-alpha; or
(f) comprises a CD3 antibody or cell surface binding fragment thereof, a CD28 antibody or cell surface binding fragment thereof, a combination of the antibody and cell surface binding fragment thereof, and a binding molecule that has the same cell surface binding specificities as the antibody; or
(g) comprises a combination of CD3 and CD28 antibodies immobilized on a bead or a surface, wherein optionally the bead or surface comprises coated beads; or
(h) comprises two or more cell surface binding molecules selected from any of (a) through (g); or
(i) comprises another molecule that is used to increase or reinforce the ability of the molecule to bind to the surface of the cell; or
(j) is complexed with another molecule; or
(k) is found on the primary cell's surface and binds to the surface of another cell.

16. The method of claim 1, wherein the conditions comprise:
(a) further incubation with a cell surface binding molecule or a cytokine; or
(b) further incubation with interleukin-2; or
(c) culturing the cells for about seven days; or
(d) culturing the cells for about 14 days.

17. The method of claim 1, wherein the lentiviral vector is:
(a) derived from a human immunodeficiency virus (HIV); or
(b) derived from HIV-1, HIV-2, or a combination thereof; or
(c) a chimeric vector comprising HIV sequences, wherein optionally the HIV sequences comprise HIV-1 and HIV-2 sequences; or
(d) VRX496 or a derivative thereof.

18. The method of claim 1, wherein said contacting occurs ex vivo in a mixed or pure cell culture, a tissue or an organ system.

19. A method to introduce a genetic material into a cell comprising ex vivo introduction of the cell transduced by the method of claim 1 into a living subject, a tissue, an organ, a blastocyst or an embryonic stem cell.

20. Use of a primary cell of the hematopoietic system or hematopoietic stem cell transduced by the method of claim 1 for the preparation of a pharmaceutical composition.

21. Use of a primary cell of the hematopoietic system or hematopoietic stem cell transduced by the method of claim 1, for the preparation of a pharmaceutical composition for the treatment or prevention of a viral infection in a subject.

22. Use of a primary cell of the hematopoietic system or hematopoietic stem cell transduced by the method of claim 1, for the preparation of a pharmaceutical composition for the treatment or prevention of an HIV infection in a subject.

23. Use of a primary cell of the hematopoietic system or hematopoietic stem cell transduced by the method of claim 1, for the preparation of a pharmaceutical composition for the treatment or prevention of cancer.

24. The use of claim 23, wherein the cancer is breast cancer or a cancer of the endothelial cells.

25. A pharmaceutical composition for gene therapy to treat or prevent an abnormality caused by a genetic defect, or to treat, diagnose, alleviate or prevent a tumor or a cancer, produced by the method of claim 1, and optionally wherein the abnormality caused by a genetic defect or tumor or cancer is a breast cancer tumor.

26. A pharmaceutical composition for gene therapy to treat or prevent an abnormality caused by an infection, produced by the method of claim 1.

27. The pharmaceutical composition of claim 26, wherein the infection is a viral infection, and optionally wherein the viral infection is a human immunodeficiency virus (HIV) infection.

28. The pharmaceutical composition of claim 25, wherein the pharmaceutical composition is formulated for use ex vivo.

29. The pharmaceutical composition of claim 26, wherein the pharmaceutical composition is formulated for use ex vivo.

30. A method for stable transduction of primary cells of the hematopoietic system and/or hematopoietic stem cells, comprising contacting, in vitro or ex vivo, the surface of the cells with both a lentiviral vector and at least one molecule which binds the cell surface, and culturing the cells in a ventilated vessel comprising two or more layers under conditions conducive to growth and/or proliferation, wherein the vessel is suitable for culturing at least about 100 million cells, and wherein the contacting of the primary cells with the cell surface molecule makes the cells more receptive to transduction by the lentiviral vector.

31. The method of claim 30, wherein the presence of the cell surface molecule results in:
(a) the cell's chromatin being more receptive to DNA integration; or
(b) integration of the lentiviral vector into a cellular site favorable for expression of a gene from the lentiviral vector; or
(c) more efficient entry of a nucleic acid containing capsid into the cytoplasm of the cells; or
(d) more efficient entry of the virus across a cell membrane or across an internal membranous structure of the cells; or
(e) the primary cells being more permissive for nuclear import of the genetic material contained in the viral vector.

32. The method of claim 1 or claim 31, wherein the cell surface binding molecule, antibody, antigen binding fragment, ligand or cell surface molecule comprises: anti-CD3 or anti-CD28 antibodies which bind the cells and make them more receptive to vector transduction; antibodies or ligands for the FLT-3 ligand, TPO, and Kit ligand receptors, which bind the cells and make them more receptive to vector transduction; antibodies or ligands for GM-CSF and IL-4 receptors, which bind dendritic cells or their precursors, monocytes, CD34 positive stem cells, or their differentiated progenitor cells on the dendritic cell lineage, and make them more receptive to vector transduction; a polypeptide, nucleic acid, carbohydrate, lipid or ion, or a polypeptide, nucleic acid, carbohydrate, lipid or ion complexed with another substance that binds CD1a, CD1b, CD1c, CD1d, CD2, CD3γ, CD3δ, CDε, CD4, CD5, CD6, CD7, CD8α, CD8β, CD9, CD10, CD11a, CD11b, CD11c, CDw12, CD13, CD14, CD15, CD15s, CD16a, CD16b, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45R, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD67, CD68, CD69, CDw70, CD71, CD72, CD73, CD74, CDw75, CDw76, CD77, CD79cc, CD79(3, CD80, CD81, CD82, CD83, CD84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CDw109, CD114, CD115, CD116, CD117, CD118, CD119, CD120a; CD120b, CD121a, CD121b, CD122, CD123, CDw124, CD125, CD126, CDw127, CDw128a, CDw128b, CDw130, CDw131, CD132, CD133, CD134, CD135, CD136, CDw137, CD138, CD139, CD140a, CD140b, CD141, CD142, CD143, CD144, CDw145, CD146, CD147, CD148, CDw149, CD150, CD151, CD152, CD153, CD154, CD155, CD156, CD157, CD158a, CD158b, CD161, CD162, CD163, CD164, CD165, CD166 or TCRζ on the cells and makes them more receptive to vector transduction.

33. A method for stable transduction of a primary cell of the hematopoietic system and/or a hematopoietic stem cell isolated from an HIV-infected subject, comprising the steps of:
(a) isolating from the HIV-infected subject primary cells of the hematopoietic system cells or hematopoietic stem cells;
(b) optionally, pre-stimulating the primary cells or hematopoietic stem cells with at least one cell surface binding molecule;
(c) contacting simultaneously in vitro or ex vivo the hematopoietic system cells or hematopoietic stem cells with a lentiviral vector and at least one cell surface binding molecule; and
(d) culturing the cells in a ventilated vessel comprising two or more layers under conditions conducive to growth and/or proliferation, wherein the vessel is suitable for culturing at least about 100 million cells.

34. A system comprising:
(a) a ventilated vessel comprising two or more layers; and
(b) isolated non-adherent primary cells of the hematopoietic system and/or hematopoietic stem cells.

35. The system of claim 34, wherein the primary cell is a lymphocyte, a precursor of a lymphocyte, a CD4 positive cell, a hematopoietic stem cell of a CD4 positive cell, a CD8 positive cell, a hematopoietic stem cell of a CD8 positive cell, a CD34 positive cell, a hematopoietic stem cell of a CD34 positive cell, a dendritic cell, a cell capable of differentiating into a dendritic cell, a human primary cell of the hematopoietic system and/or a human hematopoietic stem cell, a precursor of a human hematopoietic stem cell, an astrocyte, a skin fibroblast, a epithelial cell, a neuron, a dendritic cell, a leukocyte, a cell associated with the immune response, a vascular endothelial cell, a tumor cell, a tumor vascular endothelial cell, a liver cell, a lung cell, a bone marrow cell, an antigen presenting cell, a stromal cell, an adipocyte, a muscle cell, a pancreatic cell, a kidney cell, an ovum, a spermatocyte, a cell that contributes to the germ line, an embryonic pluripotential stem cell or a progenitor cell, a blood cell, a non-nucleated cell, a platelet cell, or an erythrocyte, or a derivative thereof.

36. The system of claim 34, wherein the multilayer vessel is rectangular in shape, square in shape, or rectangular in shape with a curved edge, or square in shape with a curved edge.

37. The method of claim 34, wherein the vessel is suitable for culturing at least about 100 million cells.
